(19) United States
(12) Patent Application Publication
(10) Pub. No.: US 2022/0390471 A1
(43) Pub. Date: Dec. 8, 2022

(54) INTEGRATED DRUG DISCOVERY PLATFORM FOR PROTEIN MISFOLDING DISORDERS ASSOCIATED WITH METABOLITE ACCUMULATION

(71) Applicant: Ramot at Tel-Aviv University Ltd., Tel Aviv (IL)

(72) Inventors: Ehud GAZIT, Tel Aviv (IL); Dana LAOR, Tel Aviv (IL); Dorin SADE YAZDI, Tel Aviv (IL)

(21) Appl. No.: 17/831,232

(22) Filed: Jun. 2, 2022

Related U.S. Application Data
(60) Provisional application No. 63/196,363, filed on Jun. 3, 2021.

(51) Int. Cl. G01N 33/68

(52) U.S. Cl. CPC G01N 33/6896; G01N 2500/00; G01N 2800/2835; G01N 2800/52; G01N 2333/4703

(57) ABSTRACT

The present disclosure provides yeast screening system/s and methods for screening of candidate therapeutic compound/s for treating, at least one proteniopathy and/or protein misfolding disorder. More specifically, the present disclosure provides systems and methods for identifying a therapeutic compound that inhibit Hcy fibril formation and uses thereof in treating Alzheimer's disease.

### TECHNOLOGICAL FIELD

The invention relates to drug discovery. More specifically, the present invention provides in vivo systems and high throughput screening platform for drugs applicable in protein misfolding disorders.

### BACKGROUND ART

References considered to be relevant as background to the presently disclosed subject matter are listed below:

Acknowledgement of the above references herein is not to be inferred as meaning that these are in any way relevant to the patentability of the presently disclosed subject matter.
1. C. Reitz, R. Mayeux, Alzheimer disease: Epidemiology, diagnostic criteria, risk factors and biomarkers. Biochem. Pharmacol. 88, 640-651 (2014).
2. 2015 Alzheimer's disease facts and figures. Alzheimer's Dement. 11, 332-384 (2015).
3. J. Meldolesi, Alzheimer's disease: Key developments support promising perspectives for therapy. Pharmacol. Res. 146, 104316 (2019).
4. S. R. Chandra, Alzheimer's disease. An alternative approach. Indian J. Med. Res. 145, 723-729 (2017).
5. F. Chiti, C. M. Dobson, Protein Misfolding, Functional Amyloid, and Human Disease. Annu. Rev. Biochem. 75, 333-366 (2006).
6. D. Eisenberg, M. Jucker, The Amyloid State of Proteins in Human Diseases. Cell 148, 1188-1203 (2012).
7. M. Citron, et al., Mutation of the β-amyloid precursor protein in familial Alzheimer's disease increases β-protein production. Nature 360, 672-674 (1992).
8. Z. Amtul, Why therapies for Alzheimer's disease do not work: Do we have consensus over the path to follow? Ageing Res. Rev. 25, 70-84 (2016).
9. E. Karran, B. De Strooper, The amyloid cascade hypothesis, are we poised for success or failure? J. Neurochem. 139, 237-252 (2016).
10. F. Pistollato, et al., Alzheimer disease research in the 21st century: Past and current failures, new perspectives and funding priorities. Oncotarget 7, 38999-39016 (2016).
11. J. Hardy, G. Higgins, Alzheimer's disease: the amyloid cascade hypothesis. Science 256, 184-185 (1992).
12. T. P. J. Knowles, M. Vendruscolo. C. M. Dobson, The amyloid state and its association with protein misfolding diseases. Nat. Rev. Mol. Cell Biol. 15, 384-396 (2014).
13. G. G. Glenner, C. W. Wong, Alzheimer's disease: initial report of the purification and characterization of a novel cerebrovascular amyloid protein. Biochem. Biophys. Res. Commun. 120, 885-890 (1984).
14. C. A. Ross, M. A. Poirier, Protein aggregation and neurodegenerative disease. Nat. Med. 10, S10-S17 (2004).
15. E. Socha, M. Koba, P. Kośliński, Amino acid profiling as a method of discovering biomarkers for diagnosis of neurodegenerative diseases. Amino Acids 51, 367-371 (2019).
16. J. Xu, et al., Graded perturbations of metabolism in multiple regions of human brain in Alzheimer's disease: Snapshot of a pervasive metabolic disorder. Biochim. Biophys. Acta-Mol. Basis Dis. 1862, 1084-1092 (2016).
17. R. Kaddurah-Daouk, et al., Metabolomic changes in autopsy-confirmed Alzheimer's disease. Alzheimer's Dement. 7, 309-317 (2011).
18. M. Kori, B. Aydin, S. Unal, K. Y. Arga, D. Kazan, Metabolic Biomarkers and Neurodegeneration: A Pathway Enrichment Analysis of Alzheimer's Disease. Parkinson's Disease, and Amyotrophic Lateral Sclerosis. Omi. A J. Integr. Biol. 20, 645-661 (2016).
19. E. Trushina, M. M. Mielke, Recent advances in the application of metabolomics to Alzheimer's Disease. Biochim. Biophys. Acta 1842, 1232-1239 (2013).
20. M. S. Morris, Homocysteine and Alzheimer's disease. Lancet Neurol. 2, 425-428 (2003).
21. F. Van Dam, W. A. Van Gool, Hyperhomocysteinemia and Alzheimer's disease: A systematic review. Arch. Gerontol. Geriatr. 48, 425-430 (2009).
22. S. Seshadri, et al., Plasma Homocysteine as a Risk Factor for Dementia and Alzheimer's Disease. N. Engl. J. Med. 346, 476-483 (2002).
23. R M. Soni, S. C. Tiwari, A. A. Mahdi, N. Kohli, Serum Homocysteine and Behavioral and Psychological Symptoms of Dementia: Is There Any Correlation in Alzheimer's Disease? Ann. Neurosci. 25, 152-159 (2018).
24. I. Sleeman, et al., Urate and Homocysteine: Predicting Motor and Cognitive Changes in Newly Diagnosed Parkinson's Disease. J. Parkinsons. Dis. 9, 351-359 (2019).
25. M. C. Bakeberg, et al., Elevated Serum Homocysteine Levels Have Differential Gender-Specific Associations with Motor and Cognitive States in Parkinson's Disease. Parkinsons. Dis. 2019, 1-8 (2019).
26. L. Baroni, et al., Association Between Cognitive Impairment and Vitamin B12, Folate, and Homocysteine Status in Elderly Adults: A Retrospective Study. J Alzheimer's Dis. 70, 441-451 (2019).
27. T. Den Heijer, et al., Homocysteine and brain atrophy on MRI of non-demented elderly. Brain 126, 170-175 (2003).
28. J. P. Kraus, et al., Cystathionine β-synthase mutations in homocystinuria. Hum. Mutat. 13, 362-375 (1999).
29. B. L. Zaric, et al., Homocysteine and Hyperhomocysteinaemia. Curr. Med. Chem. 25(2018).
30. J. Kim, H. Kim, H. Roh, Y. Kwon, Causes of hyperhomocysteinemia and its pathological significance. Arch. Pharm. Res. 41, 372-383 (2018).
31. J. Zou, et al., Trefoil Factor 3, Cholinesterase and Homocysteine: Potential Predictors for Parkinson's Disease Dementia and Vascular Parkinsonism Dementia in Advanced Stage. Aging Dis. 9, 51 (2018).
32. N. Licking, et al., Homocysteine and cognitive function in Parkinson's disease. Parkinsonism Relat. Disord. 44, 1-5 (2017).
33. P. Saadat, et al., Serum Homocysteine Level in Parkinson's Disease and Its Association with Duration, Cardinal Manifestation, and Severity of Disease. Parkinsons. Dis. 2018, 1-6 (2018).
34. A. D. Smith, et al., Homocysteine and Dementia: An International Consensus Statement. J. Alzheimer's Dis. 62, 561-570 (2018).
35. P. I. Ho, D. Ortiz, E. Rogers, T. B. Shea, Multiple aspects of homocysteine neurotoxicity: Glutamate excitotoxicity, kinase hyperactivation and DNA damage. J. Neurosci. Res. 70, 694-702 (2002).
36. I. I. Kruman, et al., Folic Acid Deficiency and Homocysteine Impair DNA Repair in Hippocampal Neurons and Sensitize Them to Amyloid Toxicity in Experimental Models of Alzheimer's Disease. J. Neurosci. 22, 1752-1762 (2002).
37. P. Pierozan, et al., Synergistic Toxicity of the Neurometabolites Quinolinic Acid and Homocysteine in Cortical Neurons and Astrocytes: Implications in Alzheimer's Disease. Neurotox. Res. 34, 147-163 (2018).
38. L. F. Agnati, et al., Aβ peptides as one of the crucial volume transmission signals in the trophic units and their interactions with homocysteine. Physiological implications and relevance for Alzheimer's disease. J. Neural Transm. 114, 21-31 (2007).
39. C. E. Zhang, et al., Hyperhomocysteinemia Increases β-Amyloid by Enhancing Expression of γ-Secretase and Phosphorylation of Amyloid Precursor Protein in Rat Brain. Am. J. Pathol. 174, 1481-1491 (2009).
40. N. Shirafuji, et al., Homocysteine Increases Tau Phosphorylation, Truncation and Oligomerization. Int. J. Mol. Sci. 19, 891 (2018).
41. L. Adler-Abramovich, et al., Phenylalanine assembly into toxic fibrils suggests amyloid etiology in phenylketonuria. Nat. Chem. Biol. 8, 701-706 (2012).
42. E. Gazit, Metabolite amyloids: a new paradigm for inborn error of metabolism disorders. J. Inherit. Metab. Dis. 39, 483-488 (2016).
43. S. Shaham-Niv, L. Adler-Abramovich, L. Schnaider, E. Gazit, Extension of the generic amyloid hypothesis to nonproteinaceous metabolite assemblies. Sci. Adv. 1, e1500137 (2015).
44. S. Shaham-Niv, et al., Formation of Apoptosis-Inducing Amyloid Fibrils by Tryptophan. Isr. J. Chem. 57, 729-737 (2017).
45. O. Tavassoly, et al., Quinolinic Acid Amyloid-like Fibrillar Assemblies Seed α-Synuclein Aggregation. J. Mol. Biol. 430, 3847-3862 (2018).
46. B. G. Anand, K. Dubey, D. S. Shekhawat, K. Kar, Intrinsic property of phenylalanine to trigger protein aggregation and hemolysis has a direct relevance to phenylketonuria. Sci. Rep. 7, 1-9 (2017).
47. D. Sade, S. Shaham-Niv, Z. A. Amon, O. Tavassoly, E. Gazit, Seeding of proteins into amyloid structures by metabolite assemblies may clarify certain unexplained epidemiological associations. Open Biol. 8, 170229 (2018).
48. D. Laor, et al., Fibril formation and therapeutic targeting of amyloid-like structures in a yeast model of adenine accumulation. Nat. Commun. 10, 1-11 (2019).
49. S. Rencus-Lazar, Y. DeRowe, H. Adsi, E. Gazit, D. Laor, Yeast Models for the Study of Amyloid-Associated Disorders and Development of Future Therapy. Front. Mol. Biosci. 6, 15 (2019).
50. S. Shaham-Niv, et al., Differential inhibition of metabolite amyloid formation by generic fibrillation-modifying polyphenols. Commun. Chem. 1, 1-11 (2018).
51. P. A. Williams, C. E. Hughes, K. D. M. Harris, L-Lysine: Exploiting Powder X-ray Diffraction to Complete the Set of Crystal Structures of the 20 Directly Encoded Proteinogenic Amino Acids. Angew. Chemie Int. Ed 54, 3973-3977 (2015).
52. C. H. Görbitz, K. Vestli, R. Orlando, A solution to the observed Z′=2 preference in the crystal structures of hydrophobic amino acids. Acta Crystallogr. Sect. B Struct. Sci. 65, 393-400 (2009).
53. C. H. Görbitz. Crystal structures of amino acids: from bond lengths in glycine to metal complexes and high-pressure polymorphs. Crystallogr. Rev. 21, 160-212 (2015).
54. S. Bera, S. Mondal, S. Rencus-Lazar, E. Gazit, Organization of Amino Acids into Layered Supramolecular Secondary Structures. Acc. Chem. Res. 51, 2187-2197 (2018).
55. T. D. Do, W. M. Kincannon, M. T. Bowers, Phenylalanine Oligomers and Fibrils: The Mechanism of Assembly and the Importance of Tetramers and Counterions. J. Am. Chem. Soc. 137, 10080-10083 (2015).
56. C. Bleiholder. N. F. Dupuis, T. Wyttenbach, M. T. Bowers, Ion mobility-mass spectrometry reveals a conformational conversion from random assembly to β-sheet in amyloid fibril formation. Nat. Chem. 3, 172-177 (2011).
57. T. D. Do, et al., Amino Acid Metaclusters: Implications of Growth Trends on Peptide Self-Assembly and Structure. Anal. Chem. 88, 868-876 (2016).
58. S. Shaham-Niv, et al., Intrinsic Fluorescence of Metabolite Amyloids Allows Label-Free Monitoring of Their Formation and Dynamics in Live Cells. Angew. Chemie—Int. Ed. 57, 12444-12447 (2018).
59. A. Šarić, T. C. T. Michaels, A. Zaccone, T. P. J. Knowles, D. Frenkel, Kinetics of spontaneous filament nucleation via oligomers: Insights from theory and simulation. J. Chem. Phys. 145, 211926 (2016).
60. W. Hwang, S. Zhang, R. D. Kamm, M. Karplus, Kinetic control of dimer structure formation in amyloid fibrillogenesis. Proc. Natl. Acad. Sci. 101, 12916-12921 (2004).
61. A. R. White, et al., Homocysteine potentiates copper- and amyloid beta peptide-mediated toxicity in primary neuronal cultures: Possible risk factors in the Alzheimer's-type neurodegenerative pathways. J. Neurochem. 76, 1509-1520 (2001).
62. B. Ono, et al., Cysteine biosynthesis in Saccharomyces cerevisiae: mutation that confers cystathionine beta-synthase deficiency. J. Bacteriol. 170, 5883-5889 (1988).
63. E. M. Weekman, A. E. Woolums, T. L. Sudduth, D. M. Wilcock, Hyperhomocysteinemia-Induced Gene Expression Changes in the Cell Types of the Brain. ASN Neuro 9, 175909141774229 (2017).
64. J. Guo, S. Ni, Q. Li, J. Z. Wang, Y. Yang, Folate/Vitamin B Alleviates Hyperhomocysteinemia-Induced Alzheimer-Like Pathologies in Rat Retina Neurosci. Bull. 35, 325-335 (2019).
65. H. Oakley, et al., Intraneuronal β-amyloid aggregates, neurodegeneration, and neuron loss in transgenic mice with five familial Alzheimer's disease mutations: Potential factors in amyloid plaque formation. J. Neurosci. 26, 10129-10140 (2006).
66. S. Kalinin, J. Richardson, D. Feinstein, A PPARdelta Agonist Reduces Amyloid Burden and Brain Inflammation in a Transgenic Mouse Model of Alzheimers Disease. Curr. Alzheimer Res. 6, 431-437 (2009).
67. R. Diaz-Arrastia, Homocysteine and Neurologic Disease. Arch. Neurol. 57, 1422-1428 (20(0)).
68. Y. H. Chung, K. M. Joo, Y. J. Lee, C. I. Cha, Immunohistochemical study on the distribution of MnSOD in the central nervous system of the transgenic mice expressing a human Cu/Zn SOD mutation. Brain Res. 990, 215-220 (2003).
69. D. Braconi, et al., Homogentisic acid induces aggregation and fibrillation of amyloidogenic proteins. Biochim. Biophys. Acta 1861, 135-146 (2017).
70. G. Román, O. Mancera-Páez, C. Bernal, Epigenetic Factors in Late-Onset Alzheimer's Disease: MTHFR and CTH Gene Polymorphisms, Metabolic Transsulfuration and Methylation Pathways, and B Vitamins. Int. J. Mol. Sci. 20, 319 (2019).
71. S. Ibrahim, S. Maqbool. M. Azam, M. P. Iqbal, R. Qamar. CBS mutations and MTFHR SNPs causative of hyperhomocysteinemia in Pakistani children. Mol. Biol. Rep. 45, 353-360 (2018).
72. M. T. Mursleen, S. Riaz, Implication of homocysteine in diabetes and impact of folate and vitamin B12 in diabetic population. Diabetes Metab. Syndr. Clin. Res. Rev. 11, S141-S146 (2017).
73. G. J. Hankey, J. W. Eikelboom, Homocysteine and vascular disease. Lancet 354, 407-413 (1999).
74. A. Pinna, F. Zaccheddu. F. Boscia, C. Carru, G. Solinas, Homocysteine and risk of age-related macular degeneration: a systematic review and meta-analysis. Acta Ophthalmol. 96, e269-e276 (2018).
75. D. Zhang. X. Wen. W. Wu, Y. Guo, W. Cui, Elevated Homocysteine Level and Folate Deficiency Associated with Increased Overall Risk of Carcinogenesis: Meta-Analysis of 83 Case-Control Studies Involving 35,758 Individuals. PLoS One 10, e0123423 (2015).
76. R. Martinez-Vega, et al., Folic acid deficiency induces premature hearing loss through mechanisms involving cochlear oxidative stress and impairment of homocysteine metabolism. FASEB J. 29, 418-432 (2015).
77. A. Altomare, et al., EXPO2013: a kit of tools for phasing crystal structures from powder data. J. Appl. Crystallogr. 46, 1231-1235 (2013).
78. B. H. Toby, R. B. Von Dreele, GSAS-II: The genesis of a modern open-source all purpose crystallography software package. J. Appl. Crystallogr. 46, 544-549 (2013).
79. WO 2020/21313.

### BACKGROUND OF THE INVENTION

Alzheimer's disease (AD) is the most common neurodegenerative disorder, currently affecting tens of millions of individuals globally and leading to immense social and financial impacts (1, 2). In spite of the vast efforts to develop a pharmacological treatment for the disease, recent clinical trials had failed (3, 4). While it is very clear that amyloid formation is associated with AD (5, 6) and genetic variations in the gene coding for the β-amyloid polypeptide are associated with early onset of the disease (7), direct targeting of the aggregation of AD-associated pathological proteins and polypeptides has so far not resulted in clinical development of any disease-modifying treatment (8-10). Therefore, there is an essential need to understand the early biological changes that may induce the diverse pathologies observed in AD and other forms of dementia in order to identify new therapeutic targets (10).

In the last decades, an enormous body of research had provided important information on the amyloid cascade, oxidative stress and inflammatory response that are involved in AD (3, 11). Specifically, the notion of β-amyloid toxicity and extracellular plaque formation as the main cause of neuronal and synaptic loss has been extensively studied (12-14). Yet, the key to AD treatment and prevention remains elusive and the initial steps leading to the formation and accumulation of these protein aggregates and the role of nonproteinaceous agents in this process are still not understood (10). Since only one percent of AD cases results from a familial mutation and the majority of patients are sporadic (2, 4), it is important to explore new directions to understand the biological mechanisms underlying these cases. Studies of metabolite profiling in brain regions and body fluids of AD and Parkinson's disease (PD) patients show an interference with specific metabolic pathways (15-19). Homocysteine (Hcy), a non-coded amino acid, was identified as a major risk factor for AD as high plasma concentrations were associated with the progression of the disease (20-22). Higher Hcy serum concentration was also correlated with behavioral and psychological symptoms of AD (23) and was associated with change in motor function and cognitive decline in PD as well as with a more severe cognitive impairment in elderly adults (24-26). Furthermore, it was shown that significantly decreased hippocampal and cortical volume is associated with increased Hcy plasma concentration (27).

The hyperhomocysteinemia inborn error of metabolism (IEM) disorder results from cystathionine β-synthase (CBS) deficiency, leading to excess of Hcy, and is characterized by severe cognitive consequences (28-30). While elevated plasma Hcy is frequently reported as a strong and independent risk factor for the development of cognitive decline and dementia, the mechanism of its involvement is elusive (20, 31-34). Interestingly, the association between Hcy and AD-related pathological proteins was also demonstrated. Hcy-rich medium was shown to be cytotoxic to hippocampal and cortical neurons, resulting in increased β-amyloid-induced cell death (35-37). In addition, Hcy was found to bind β-amyloid₁₋₄₀, thereby stimulating β-sheet structure formation to facilitate its deposition. Indeed, induced Hcy accumulation in the brains of rats caused an elevation of β-amyloid deposition (38, 39). Furthermore, Hcy increased total tau and phosphorylated tau protein levels as well as the level of tau oligomers (40). Although the involvement of Hcy accumulation in AD pathology is evidential, no mechanistic insight has so far been suggested.

The inventors have previously demonstrated that small metabolites can self-assemble into amyloid-like structures with amyloidogenic characteristics (41-44). The presence of metabolite assemblies in IEM disorders (for example phenylalanine in Phenylketonuria) exemplifies their physiological importance in pathologically diversified diseases. Interestingly, recent studies demonstrated that metabolite assemblies could cross-seed the aggregation of proteins under physiological conditions, thereby suggesting a possible mechanism in which accumulated metabolites interfere with protein function and folding (45, 46). Assemblies of quinolinic acid, an endogenous neuro-metabolite that is involved in the pathology of PD, induce the aggregation of α-synuclein both in-vitro and in cell culture (45). In addition, phenylalanine pre-formed fibrils were shown to initiate the aggregation of several proteins under physiological conditions (46). Metabolite accumulation, structure formation and seeding of proteins may thus underlie the unknown role of metabolites in neurodegenerative pathologies (47). Specifically, seeding of amyloidogenic proteins by preformed fibrils may be part of the mechanisms underlying the stereotypical spreading of toxic aggregates in the brains of AD patients.

The inventors recently established the first in-vivo yeast model for IEM disorders by genetically modifying the yeast to reflect the mutations found in patients showing accumulation of the adenine nucleobase and its derivatives (48). Yeast model systems provide a powerful platform to elucidate the pathophysiology of diseases, as well as for the screening and the development of disease-modifying therapeutics (49, 79). This first-of-a-kind model was found to be a valid system, as supported by its robust sensitivity to adenine feeding, and by the fact that adenine supramolecular structures could be detected. Furthermore, the addition of a generic fibrillation-modifying polyphenolic compound rescued the toxic effect without lowering the concertation of adenine, indicating the therapeutic potential of the disclosed model for the modulation of structure formation (48, 50). Aiming to explore the role of Hcy in AD, the present disclosure provide the first demonstration for the formation of amyloid-like fibrils by Hcy in-vitro and in-vivo in a yeast model. Structural characterization of the Hcy fibrils and their cytotoxic effect were both studied. In addition, the inventors revealed that polyphenolic inhibitors could rescue the toxic effect of Hcy assemblies and inhibit its structure formation. Remarkably, immunohistochemistry allowed the detection of Hcy fibrils in the brain of AD model mice as well as the apparent interplay between Hcy and β-amyloid. Finally, the inventors demonstrated the cross-seeding of AD-related pathological protein by Hcy assemblies. The present disclosure therefore suggests a new research direction for the association between metabolite accumulation and the initiation of neurodegenerative processes, thus offering a new path for the development of therapeutic treatments that will target the key early stages of the disease.

### SUMMARY OF THE INVENTION

A first aspect of the present disclosure relates to a yeast screening system for candidate therapeutic compound/s for treating, preventing, ameliorating, reducing or delaying the onset of at least one proteinopathy, and/or protein misfolding disorder. The system disclosed herein comprises: (a) a yeast cell and/or yeast cell line, and/or yeast cell population, that display accumulation of at least one metabolite. It should be noted that in some embodiments at least one of: (i) the yeast cell/s carry at least one manipulation and/or modification in at least one yeast metabolic pathway that leads to accumulation of said metabolite; (ii) the yeast cell/s grow under conditions that result in accumulation of said metabolite; (iii) the yeast cell/s endogenously and/or exogenously express at least one pathological protein associated with the protein misfolding disorder. In some further embodiments the system of the present disclosure may optionally further comprises (b), at least one reagent or means for determining at least one of, the accumulation of the metabolite and at least one phenotype associated with accumulation of the metabolite and/or the accumulation of the pathologic protein.

A further aspect of the preset disclosure relates to a screening method of candidate therapeutic compounds for treating, preventing, ameliorating, reducing or delaying the onset of at least one proteinopathy and/or protein misfolding disorder. More specifically, the method comprising the steps of: in a first step (a), contacting a manipulated yeast cell and/or yeast cell line, and/or yeast cell population that display accumulation of at least one metabolite, with a candidate compound. In some embodiments, wherein at least one of: (i) the yeast cell/s carry at least one manipulation and/or modification in at least one yeast metabolic pathway that leads to accumulation of the metabolite; (ii) the yeast cell/s grow under conditions that result in accumulation of the metabolite; (iii) the yeast cell/s endogenously and/or exogenously express at least one pathological protein associated with the proteinopathy and/or protein misfolding disorder.

The next step (b), involves determining in the contacted cells of step (a), at least one of, the accumulation of the metabolite and the level of at least one phenotype associated with the accumulation of the metabolite and/or accumulation of the pathological protein. The next step (c), involves determining that the candidate is a therapeutic compound for the protein misfolding disorder if a change and/or modulation is observed in at least one of: the accumulation of the metabolite and/or accumulation of the pathological protein and/or the phenotype, in cells contacted with the candidate compound as compared with the accumulation of the metabolite, and/or accumulation of the pathological protein and/or the phenotype in the absence of the candidate compound.

A further aspect of the present disclosure relates to a screening method for candidate therapeutic compounds for treating, preventing, ameliorating, reducing or delaying the onset of a disorder characterized by at least one of beta-amyloid protein aggregation. In some embodiments, the method comprising the steps of; First step (a), involves contacting a yeast cell and/or yeast cell line, and/or yeast cell population, that display accumulation of Homocysteine, with a candidate compound.

More specifically, at least one of: (i) the yeast cell/s carry at least one manipulation in at least one yeast metabolic pathway that leads to accumulation of the homocysteine (Hcy); (ii) the yeast cell/s grow under conditions that result in accumulation of the Hcy; (iii) the yeast cell/s endogenously and/or exogenously express the beta-amyloid protein or any pathological protein associated with the disorder.

The next step (b), involves determining in the contacted cells of (a) at least one of, the accumulation of said Hcy, and/or beta-amyloid protein aggregation, and/or at least one phenotype associated with the accumulation of the at least one of Hcy and any derivative thereof. Still further, the next step (c), involves determining that the candidate is a therapeutic compound for the disorder characterized by at least one of beta-amyloid protein aggregation if a change and/or modulation is observed and/re detected in at least one of: the accumulation of said Hcy, and/or the beta-amyloid protein aggregation/accumulation, and/or the phenotype in cells treated with the compound as compared with the accumulation of the Hcy and/or the beta-amyloid protein aggregation and/or accumulation, and/or the phenotype, in the absence of the candidate compound.

A further aspect of the present disclosure provides a method for treating, preventing, ameliorating, reducing or delaying the onset of at least one proteinopathy, and/or protein misfolding disorder. More specifically, the method disclosed herein comprises the following steps: First step (I), obtaining a compound that modulates the level of at least one phenotype associated with the accumulation of at least one metabolite by a screening method. In some particular embodiments, the screening method comprises: First (a), contacting a manipulated and/or modified yeast cell and/or yeast cell line, and/or yeast cell population that display accumulation of at least one metabolite, with a candidate compound, wherein at least one of; (i) the yeast cell/s carry at least one manipulation in at least one yeast metabolic pathway that leads to accumulation of the metabolite; (ii) the yeast cell/s grow under conditions that result in accumulation of the metabolite; (iii) the yeast cell/s endogenously and/or exogenously express at least one pathological protein associated with the protein misfolding disorder. Next in step (b), determining in the contacted cells of (a) at least one of, the accumulation of the metabolite and the level of at least one phenotype associated with the accumulation of the metabolite and/or misfolding of the pathological protein. The next step (c), involves determining that the candidate is a therapeutic compound for the proteinopathy, and/or the protein misfolding disorder if a change and/or modulation is determined and/or observed in at least one of, the accumulation of the metabolite and/or accumulation of the pathological protein and/or the phenotype, is modulated as compared with the accumulation of the metabolite, and/or accumulation of the pathological protein and/or the phenotype in the absence of the candidate compound.

The next step of the therapeutic methods disclosed herein involves (II), administering a therapeutic effective amount of the compound obtained by step (I) to a subject suffering from the at least one protein misfolding disorder.

A further aspect of the present disclosure relates to a therapeutic compound for treating, preventing, ameliorating, reducing or delaying the onset of at least one proteinopathy, and/or protein misfolding disorder. In some embodiments, the compound is identified by a method comprising the steps of: (a), contacting a manipulated and/or modified yeast cell and/or yeast cell line, and/or yeast cell population that display accumulation of at least one metabolite, with a candidate compound, wherein at least one of: (i) the yeast cell/s carry at least one manipulation in at least one yeast metabolic pathway that leads to accumulation of said metabolite; (ii) the yeast cell/s grow under conditions that result in accumulation of the metabolite: (iii) the yeast cell/s endogenously and/or exogenously express at least one pathological protein associated with the protein misfolding disorder; (b), determining in the contacted cells of (a) at least one of, the accumulation of the metabolite and the level of at least one phenotype associated with the accumulation of said metabolite and/or accumulation of the pathological protein; and (c), determining that the candidate is a therapeutic compound for the proteinopathy and/or protein misfolding disorder if a change and/or modulation is detected and/or observed in at least one of, the accumulation of the metabolite and/or misfolding of the pathological protein and/or the phenotype is modulated as compared with the accumulation of the metabolite, and/or misfolding of the pathological protein and/or the phenotype in the absence of said candidate compound. Still further aspect of the present disclosure relates to a method for detection, and/or monitoring of at least one protein misfolding disorder or proteniopathy in a subject. The diagnostic method of the present disclosure comprises: in a first step (a), contacting at least one biological sample of the subject with at least one antibody specific for at least one metabolite-fibrils. The next step (b), involves classifying the subject as a subject affected by the at least one protein misfolding disorder or proteniopathy, if the metabolite-fibril is detected in the sample. In some optional embodiments, the diagnostic method may further comprise a therapeutic step (c), involving administering to a subject classified as affected by said at least one protein misfolding disorder or proteniopathy, a therapeutically effective amount of an anti-proteinopathy or At least one anti-protein misfolding disorder therapeutic agent. The present disclosure further provides a diagnostic kit for the diagnosis and monitoring of proteinopathies and/or protein misfolding disorders in a subject. The disclosed kit comprises any of the metabolite-fibril specific antibodies, or any affinity molecule specific for the metabolite-fibril/s.

A further aspect of the present disclosure method for treating, preventing, ameliorating, reducing or delaying the onset of at least one proteinopathy and/or protein misfolding disorder, the method comprising the steps of: first step (a), involves contacting at least one biological sample of the subject with at least one antibody specific for at least one metabolite-fibrils. The next step (b), involves classifying the subject as a subject affected by the at least one protein misfolding disorder or proteniopathy, if the metabolite-fibril is detected in the sample. The next step (c), involves administering to a subject classified as affected by the at least one protein misfolding disorder or proteniopathy, a therapeutically effective amount of at least one anti-proteinopathy or an anti-protein misfolding disorder therapeutic agent.

These and further aspects of the present disclosure will become apparent by the hand of the following disclosure.

### BRIEF DESCRIPTION OF THE DRAWINGS

The patent or application file contains at least one drawing executed in color. Copies of this patent or patent application publication with color drawings will be provided by the Office upon request and payment of the necessary fee.

In order to better understand the subject matter that is disclosed herein and to exemplify how it may be carried out in practice, embodiments will now be described, by way of non-limiting example only, with reference to the accompanying drawings, in which:

In order to better understand the subject matter that is disclosed herein and to exemplify how it may be carried out in practice, embodiments will now be described, by way of non-limiting example only, with reference to the accompanying drawings, in which:

FIG. 1A-1F: Characterization of Hcy assemblies

FIG. 1A. Skeletal formula of Hcy in a ball and stick model. The carbon atoms are represented in grey and nitrogen, oxygen and sulfur heteroatoms are shown in blue, red and yellow, respectively. Hydrogen atoms are omitted for clarity.

FIG. 1B. TEM micrographs of pre-formed samples of Hcy assemblies (2 mg/mL).

FIG. 1C, cryo-TEM micrographs of pre-formed samples of Hcy assemblies (2 mg/mL).

FIG. 1D-1F. Single crystal structure of Hcy.

FIG. 1D. The solid-state conformation in the asymmetric unit.

FIG. 1E (i-iii). i. Side view of the H-bonded single sheet in the b-direction. ii. Formation of a single layer by two adjacent sheets through intermolecular H-bonding. iii. Layer-by-layer structure formation by hydrophobic interactions resembles supramolecular β-sheet structure. The hydrophobic and hydrophilic regions are indicated by arrows.

FIG. 1F. Top view of the single sheet. Hydrogen bonds are shown as black dotted lines.

FIG. 2A-2B: TEM micrographs of pre-formed samples of Hcy assemblies

FIG. 2A-2B, show TEM micrographs of pre-formed samples of Hcy assemblies. FIG. 2A. 0.2 mg/mL; FIG. 2B. 10 mg/mL. Scale bar: 2 μm.

FIG. 3A-3B: IMS-MS analysis of Hcy self-assembly

FIG. 3A (i-v). Representative mass spectral data of Hcy (0.13 mg/mL in 20 mM ammonium acetate, pH 7) showing various oligomers. (i-v). Isotope distributions of some oligomers are shown.

FIG. 3B (i-ii). 2D plots of m/z vs. drift time (ms) for (i) m/z 1345-1350 showing overlapping isotope patterns of n/z=10/1, 20/2, and 30/3, and (ii) m/z 939-943 showing two features with the same isotope spacing.

FIG. 4A-4B: IMS-MS analysis of Hcy self-assembly

FIG. 4A. Comparison of experimental data and theoretical CCSs from five different models.

FIG. 4B. Representative oligomers of the monolayer, bilayer, single tube and double tube models.

FIG. 5A-5F: Kinetic analysis of Hcy self-assembly and inhibition by polyphenols

FIG. 5A. Kinetic analysis of Hcy self-assembly at the indicated concentrations detected using a ThT binding assay. ThT emission data at 480 nm (excitation at 440 nm) were measured over time.

FIG. 5B. Kinetic analysis of Hcy self-assembly at the indicated concentrations detected by auto-fluorescence intensity over time (excitation at 375 nm, emission at 450 nm).

FIG. 5C. Hcy self-assembly at different concentrations. Hcy was dissolved to a final concentration of 1 mg/mL to 12 mg/mL. Using a ThT binding assay, the fluorescence emission endpoint was measured at 480 nm (excitation at 450 nm).

FIG. 5D. Inhibition of Hcy self-assembly by polyphenols. Hcy at the indicated concentrations was mixed with the inhibitors (1 mM EGCG or 0.1 mM TA) at timepoint zero, or with PBS as a control, followed by addition of ThT. ThT emission data at 480 nm (excitation at 440 nm) were measured over time.

FIG. 5E-5F. TEM micrographs of pre-formed solutions of Hcy with polyphenols. (e) Hcy 2 mg/mL+1 mM EGCG. (f) Hcy 2 mg/mL+0.1 mM TA. Scale bar: 1 μm.

FIG. 6A-6C: kinetics of Hcy self-assembly

FIG. 6A. Kinetic analysis of Hcy self-assembly at the indicated concentrations detected using a ThT binding assay.

FIG. 6B. methionine self-assembly at the indicated concentrations detected using a ThT binding assay. ThT emission data at 480 nm (excitation at 440 nm) were measured over time.

FIG. 6C. TEM micrograph of a pre-formed solution of methionine as a control.

FIG. 7A-7D. Cytotoxicity of Hcy assemblies

FIG. 7A. Cytotoxicity of Hcy assemblies as determined by MTT cell viability assay. Treated SH-SY5Y cells were incubated with pre-formed Hcy assemblies prepared in cell medium, for 24 hours. The control reflects medium without Hcy, which was treated in the same manner. Absorbance was determined at 570/680 nm.

FIG. 7B. Cytotoxicity of Hcy assemblies tested following self-assembly inhibition by polyphenols and cytotoxicity of methionine as a control. Hcy assemblies (2 mg/mL) were similarly prepared in cell medium in the presence of EGCG (0.1 mM) or TA (0.01 mM). Methionine solution was similarly prepared in cell medium. The control reflects medium with no metabolites, which was treated in the same manner. Treated SH-SY5Y cells were incubated with the solutions for 24 hours followed by MTT cell viability assay. (**P<0.01, ***P<0.001 Student's t-test).

FIG. 7C-7D. Apoptotic activity studied by annexin V and propidium iodide (PI) assay. SH-SY5Y cells were treated as described for MTT assay. After incubation, annexin V-FITC and P1 were added to the cells, followed by flow cytometry analysis using a single laser-emitting excitation light at 488 nm. FIG. 7C Flow cytometry plots of the annexin V-FITC/PI double-staining assay. Q1, PI(+) (cells undergoing necrosis); Q2, annexin V-FITC(+) PI(+) (cells in late apoptosis and undergoing secondary necrosis); Q3, annexin V-FITC(+) PI(−)(cells in early apoptosis); Q4, annexin V-FITC(−) PI(−)(live cells). FIG. 7D Quantification of the flow cytometry results, apoptosis is represented in blue (early+late apoptosis) and necrosis in gray.

FIG. 8A-8B: Cytotoxicity of Hcy assemblies

FIG. 8A. HEK293 cells. The solutions were prepared as described for SH-SY5Y.

FIG. 8B. SH-SY5Y cells, following self-assembly inhibition by polyphenols and the inhibitors alone as a control. Hcy assemblies (2 mg/mL) were prepared in cell medium in the presence of EGCG (0.1 mM) or TA (0.01 mM). EGCG and TA control solutions were prepared in cell medium. The controls reflect medium with no metabolites, which was treated in the same manner. Treated SH-SY5Y or HEK293 cells were incubated with the solutions for 24 hours followed by MTT cell viability assay.

FIG. 9: Detection of amyloid-like structures in treated SH-SY5Y cells

Representative confocal and differential interference contrast images of treated and untreated cells using ProteoStat staining. Cells were treated for 4 hours with Hcy assemblies (2 mg/mL) prepared in cells media. The control reflects medium which was treated in the same manner. The scale bar: 10 μm.

FIG. 10A-10F: Yeast model for Hcy accumulation and toxicity

FIG. 10A. WT and cys4Δ strains were serially diluted and spotted on YPD, SD medium containing 13 mg/L cysteine and 100 mg/L Hcy (SD+cys+Hcy), SD medium without cysteine and Hcy (SD-cys) or SD medium containing 13 mg/L cysteine without Hcy (SD+cys).

FIG. 10B-10C. Growth curves of (FIG. 10B) WT and (FIG. 10C) cys4Δ on SD media with or without Hcy at different concentrations (40 mg/L, 80 mg/L and 10 mg/L).

FIG. 10D-10E. Flow cytometry analysis of (FIG. 10D) WT and (FIG. 10F) cys4Δ strains grown in SD media containing 13 mg/L cysteine and 100 mg/L Hcy or without Hcy, using ProteoStat staining. The graphs represent quantification of the flow cytometry results presented in the histograms.

FIG. 10F. WT and cys4Δ strains diluted to OD₆₀₀ 0.01 were grown in SD media containing 13 mg/L cysteine and 100 mg/L Hcy, with or without 0.3 mM TA. The percentage of growth was calculated as the growth under the indicated condition compared to the growth of WT without TA (*P<0.05, ***P<0.001 Student's t-test).

FIG. 11: In-vivo formation of amyloid-like structures upon Hcy feeding Representative confocal and differential interference contrast images of WT and cys4Δ cells under the indicated conditions using ProteoStat staining. Scale bar: 2.5 μm

FIG. 12A-12B: Detection of Hcy fibrils, tested using two commercial antibodies (from Abcam and Sigma, see Methods) and the anti-Hcy-fibrils antibody generated in this study (RαHcy).

FIG. 12A. ELISA assay (**P<0.01, ***P<0.001 Student's t-test).

FIG. 12B. AD model mice. Top: Brain sections stained with one of the three anti-Hcy antibodies, as indicated (green). Bottom: The same sections stained with DAPI (blue) and merged with Hcy signal. Scale bar: 200 μm.

FIG. 13A-13F: Detection of Hcy fibrils correlates with β-amyloid₁₋₄₂ aggregation in AD model mice

Images represent sections of 5×FAD or WT animals.

FIG. 13A-13B. Sections stained with anti-Hcy-fibrils antibodies (green).

FIG. 13C-13D. Sections stained with anti β-amyloid₁₋₄₂ antibody 6E10 (red).

FIG. 13E-13F. Merge of Hcy and β-amyloid₁₋₄₂ staining.

FIGS. 13E′-13F′. Enlarged images of (FIG. 13E) and (FIG. 13F), respectively, merged with DAPI staining. Scale bar: 200 μm.

FIG. 14A-14D: Detection of Hcy fibrils correlates with β-amyloid₁₋₄₂ in AD model mice

The figure shows two additional and different animals brain sections co-stained with anti-Hcy-fibrils antibodies (green), anti β-amyloid₁₋₄₂ antibody 6E10 (red) and DAPI (blue). Scale bar: 200 μm.

FIGS. 14A, 14B. show brain sections of WT mice.

FIGS. 14C, 14D. show brain sections of 5×FAD mice.

FIG. 15A-15F: Detection of Hcy fibrils and co-localization with astrocytes in AD model mice

Images represent sections of mice stained with anti-Hcy-fibrils antibodies (green) or anti-GFAP antibodies (red).

FIGS. 15A-15C. disclose brain sections of 5×FAD mice. Sections in FIG. 15A were stained with anti-Hcy-fibrils antibodies (green), FIG. 15B stained with anti-GFAP antibodies (red), and FIG. 15C presents Merge of Hcy and GFAP staining with DAPI staining. Scale bar: 200 μm.

FIGS. 15D-15F. disclose brain sections of WT mice. Sections in FIG. 15D were stained with anti-Hcy-fibrils antibodies (green), FIG. 15E stained with anti-GFAP antibodies (red), and FIG. 15F presents Merge of Hcy and GFAP staining with DAPI staining.

FIG. 16: Cross-seeding of β-amyloid₁₋₄₂ by Hcy assemblies

Pre-formed Hcy fibrils were added at 20% v/v (stock solutions of 2, 5, 10 mg/mL) to 3 μM β-amyloid₁₋₄₂ monomers solution. Aggregation was monitored using a ThT binding assay. ThT emission data at 480 nm (excitation at 450 nm) were measured over time. Blank measurements of the same reaction without the protein were respectively subtracted.

### DETAILED DESCRIPTION OF THE INVENTION

High levels of homocysteine are reported as a risk factor for Alzheimer's disease (AD). Correspondingly, inborn hyperhomocysteinemia is associated with an increased predisposition for the development of dementia in later stages of life. Yet, the mechanistic link between homocysteine accumulation and the pathological neurodegenerative processes is still elusive. Furthermore, in spite of the clear association of protein aggregation and Alzheimer's disease, attempts for the development of therapy that specifically targets this process were not successful. It is envisioned that the failure in the development of efficacious therapeutic intervention may lie in the metabolomic state of affected individuals. The inventors recently demonstrated the ability of metabolites to self-assemble and cross-seed the aggregation of pathological proteins, suggesting a role for metabolite structures in the initiation of neurodegenerative diseases. The present disclosure provides the first report of homocysteine crystal structure and self-assembly into amyloid-like toxic fibrils, their inhibition by polyphenols and their ability to seed the aggregation of the AD-associated β-amyloid polypeptide. A yeast model of hyperhomocysteinemia indicates a toxic effect, correlated with increased intracellular amyloid staining that could be rescued by polyphenol treatment. Analysis of AD mouse model brain sections indicates the presence of homocysteine assemblies and the interplay between pi-amyloid and homocysteine. This work implies a molecular basis for the association between homocysteine accumulation and AD pathology, potentially leading to a paradigm-shift in the understanding of AD initial pathological processes. Described herein, for the first time, the formation of amyloid-like structures by Hcy, a sulfur-containing non-coded amino acid, which is involved in numerous maladies including neurodegenerative diseases (20, 30, 67). These results decipher the previously unknown X-ray structure of Hcy and present a characterization of its self-assembly into cytotoxic amyloid-like fibrils. A yeast model of Hcy aggregation and toxicity was further established. Notably, the in-vivo yeast model recapitulates both the cell toxicity observed in cultured human cells and the rescue of toxicity by known inhibitors of amyloid formation. The yeast model may thus serve as a platform to screen for potential inhibitory compounds using high throughput screening methods (49). Finally, the presence of Hcy assemblies was demonstrated in brain sections of AD model mice, but not in the brains of WT animals, in co-localization with astrocytes, as well as their correlation with β-amyloid₁₋₄₂ aggregation. This is consistent with the previously reported occurrence of Hcy in astrocytes but provides the first immunohistochemical indication for the aggregative state of the metabolite (68). Following this striking observation, and as the epidemiological association between Hcy and AD-related pathological proteins is well-established (35, 36, 38, 39, 61), the ability of Hcy assemblies to cross-seed the aggregation of β-amyloid₁₋₄₂ was further investigated. Indeed, an induced aggregation of the pathological protein could be observed. This result is in agreement with other observations regarding protein aggregation seeded by metabolite assemblies (45, 46, 69). The data of the present disclosure suggest a dual role for Hcy assemblies in AD pathology. First. Hcy fibrils may induce cytotoxicity via apoptotic cell death. Second, Hcy assemblies may cross-seed the aggregation of β-amyloid₁₋₄₂, resulting in the formation of toxic protein amyloid structures. The interplay between the two mechanisms should be further examined, as well as the effect of inhibitory compounds impeding Hcy assembly. The present disclosure proposes therefore metabolite-protein cross-talk as an interesting possible mechanism for the initiation of neurodegenerative pathologies.

CBS and 5,10-methylenetetrahydrofolate reductases (MTHFR) are key enzymes in Hcy metabolism. Mutations and polymorphism in the genes encoding these enzymes are common in the Caucasian population, reaching up to 2%-10% (28, 67, 70, 71). Subjects carrying these alleles have an elevated risk to develop AD yet are considered “sporadic” despite the relevant genetic background. Along with AD, high levels of Hcy are involved in many other diseases, such as diabetes (72), neurological diseases (67), vascular diseases (73), age-related macular degeneration (74), cancer (75) and hearing loss (76). Thus, the formation of amyloid-like fibrils by Hcy, their toxicity and cross-seeding capability are highly relevant to many biological and medicinal fields. Additional study of the presence of Hcy fibrils (for example in human body fluids) and their role in diverse pathological processes is essential and may contribute to the identification and prevention of AD and many other illnesses.

Thus, a first aspect of the present disclosure relates to a yeast screening system for candidate therapeutic compound/s for treating, preventing, ameliorating, reducing or delaying the onset of at least one proteinopathy and/or protein misfolding disorder. The system disclosed herein comprises: (a) a yeast cell and/or yeast cell line, and/or yeast cell population, that display accumulation of at least one metabolite. It should be noted that in some embodiments at least one of; (i) the yeast cell/s carry at least one manipulation in at least one yeast metabolic pathway that leads to accumulation of the metabolite: (ii) the yeast cell/s grow under conditions that result in accumulation of the metabolite; (iii) the yeast cell/s endogenously and/or exogenously express at least one pathological protein associated with the protein misfolding disorder, and/or proteinopathy. In some further embodiments the system of the present disclosure may optionally further comprises (b), at least one reagent or means for determining at least one of, the accumulation of the metabolite and at least one phenotype associated with accumulation of the metabolite, and/or of said pathologic protein.

As noted above, the first and essential element or component of the system of the invention, as well as for the method disclosed herein, is a yeast cell and/or cell line, specifically, a manipulated and/or genetically or epigenetically modified yeast cell/s and/or yeast cell line/s, and/or yeast cell population/s, and/or any progeny thereof, more specifically, manipulated and/or modified cell/s, specifically, genetically and/or epigenetically manipulated and/or modified cells.

Yeasts are eukaryotic, single-celled microorganisms classified as members of the fungus kingdom. Yeasts are unicellular organisms with some species having the ability to develop multicellular characteristics by forming strings of connected budding cells known as pseudohyphae or false hyphae. Yeast sizes vary greatly, depending on species and environment, typically measuring 3-4 μm in diameter, although some yeast strains can grow to 40 μm in size.

Yeasts do not form a single taxonomic or phylogenetic grouping. The term “yeast” is often taken as a synonym for Saccharomyces cerevisiae, but the phylogenetic diversity of yeasts is shown by their placement in two separate phyla, the Ascomycota and the Basidiomycota. The budding yeasts (“true yeasts”) are classified in the order Saccharomycetales, within the phylum Ascomycota.

Still further. Yeast, e.g., the baker's yeast Saccharomyces cerevisiae, has significant advantages as an experimental system. Yeast are straightforward to culture and maintain, have a short generation time, and are highly genetically tractable, meaning that they can be genetically modified, rapidly, predictably, and with high precision using well known and available techniques and reagents, and are amenable to high throughput chemical and genetic screens. Minimal genetic and epigenetic variation within strains contributes to screen reproducibility. Extensive genetic and protein interaction analysis in yeast means that considerable information regarding the yeast interactome, i.e., the set of physical interactions among molecules in a cell and interactions among genes, i.e., genetic interactions, in yeast cells is available. Molecular interactions can occur between molecules belonging to different biochemical families (proteins, nucleic acids, lipids, carbohydrates, etc.) and also within a given family (e.g., nucleotide-nucleotide interactions). While yeast cells lack the complexity of a multicellular organism the highly conserved genome and eukaryotic cellular machinery that they share with human cells affords the possibility of understanding basic cell-autonomous mechanisms and physical and genetic interactions underlying complex disease processes. There are several genus of yeast. More specifically, the yeast may be one that belongs to the genus Saccharomyces, the genus Zygosaccharomyces, the genus Pichia, the genus Kluyveromyces, the genus Candida, the genus Shizosaccharomyces, the genus Issachenkia, the genus Yarrowia, or the genus Hansenula.

The yeast belonging to the genus Saccharomyces may be, for example, S. cerevisiae, S. bayanus, S boulardii. S. bulderi, S. carioanus, S. cariocus, S. chevaliers, S. dairenensis, S. ellipsoideus, S. eubayanus, S. exiguus, S. florentinus, S. kluyveri, S. martiniae, S. monacensis, S. norbensis, S. paradoxus, S. pastorianus, S. spencerorum, S. turicensis, S. unisporus, S. uvarum, or S. zonatus.

In some specific embodiments, the yeast cell and/or yeast cell line, and/or yeast cell population, and/or any progeny thereof, encompassed by the system and/or methods of the invention may be Saccharomyces cerevisiae or any strain or isolate thereof. Saccharomyces cerevisiae also known as the Baker's yeast is a species of yeast. It is known to convert by fermentation carbohydrates to carbon dioxide and alcohols used in baking and for alcoholic beverages. It is also a centrally important model organism in modern cell biology research and is one of the most thoroughly researched eukaryotic microorganisms. S. cerevisiae cells are round to ovoid, 5-10 μm in diameter. It reproduces by a division process known as budding. Specific examples of Saccharomyces strains that may be used in the invention may include S. boulardii 17 (Sb) (ATCC® MYA796™), S. cerevisiae UFMG A-905 (905), S. cerevisiae Sc47 and S. cerevisiae L11 (L11), S. cerevisiae BY4741 (ATCCX Number: 201388™), S. cerevisiae BY4743 (ATCC® 201390™), YPS128 strain, NCYC3290 strain. K11 strain, YB210 strain, CEN.PK strain (ATCC® MYA1108™), PE-2 strain, BG-1 strain (ATCC® 204700™), and derivatives thereof.

In yet some further specific embodiments, the yeast cells applicable in the systems of the invention may be the BY4741 strain. BY4741 (GenBank: RIS00000000 or GSM1312317, ATCC®) Number: 201388™), also referred to as ATCC 4040002, with the deposited Name: Saccharomyces cerevisiae Hansen, having the Genotype: MATa his3delta1 leu2delta0 met15delta0 ura3delta0, is part of a set of deletion strains derived from S288C in which commonly used selectable marker genes were deleted by design in order to minimize or eliminate homology to the corresponding marker genes in commonly used vectors without significantly affecting adjacent gene expression. The yeast strains were all directly descended from FY2, which is itself a direct descendant of S288C. Variation between BY4741 and S288C is miniscule. BY4741 was used as a parent strain for the international systematic Saccharomyces cerevisiae gene disruption project.

It should be understood that the invention provides yeast cell and/or yeast cell line, and/or yeast cell population, and/or any progeny thereof. More specifically, the invention provides yeast cell line that are in some embodiments, cell culture that is derived from one cell or set of cells of the same type (e.g., manipulated in the specific metabolic pathway) and in which under certain conditions the cells proliferate indefinitely. The invention further encompasses any cell population comprising the yeast cells of the invention, or any cell derived therefrom, where at least 10% or more, 25%, 30%, 40%, 50%, 60%, 70%, 80%, 90% or more and preferably, 100%, of the cells in the population are the modulated yeast cells of the invention. It is understood that such cell/s and progeny thereof refer not only to the particular subject cells but to the progeny or potential progeny of such a cell, specifically, any cell derived from such cell. Because certain modification may occur in succeeding generation due to either mutation or environmental influences, such progeny may not, in fact, be identical to the parent cell, but are still included within the scope of the term as used herein.

In yet some further embodiments, the yeast cells of the systems provided by the invention may be a manipulated and/or modified yeast cell/s that carry at least one manipulation and/or modification in at least one gene involved with at least one yeast metabolic pathway. In some embodiments, such manipulation is a genetic manipulation, for example, a mutation, deletion, insertion, rearrangement and the like. In some embodiments, these cells carry at least one mutation or any other manipulation and/or modification in at least one yeast gene involved in the metabolic pathway, leading to accumulation of the metabolite. As firstly shown by the present disclosure, initiates aggregation and fibril formation of at least one protein associated with the protein misfolding disorder. It should be understood that the manipulation may be either stable specifically affecting the genome of the cells and passing in any cell divisions and passages, or transient. In transient manipulation it is meant that the modification and/or modification involved are not stable and therefore are not maintained in the next generation. Such manipulation may be achieved for example by contacting the cells with a modulator that may be a genetic element, a polypeptide or any other modulator (e.g., repressor or activator) that targets or is directed at a nucleic acid sequence encoding a product that is directly or indirectly participating in the metabolic pathway involved with the metabolite accumulated in the protein misfolding disorder. In yet some further embodiments, the yeasts cell/s, cell lines, yeast cell populations or any progeny thereof, may be alternatively or additionally manipulated by at least one epigenetic manipulation and/or modification.

The yeast cells provided by the systems of the invention contain or carry at least one manipulation and/or modification in at least one yeast metabolic pathway. Such manipulation leads to accumulation of the specific metabolite that have been shown by the present disclosure as participating in aggregation of pathogenic proteins associated with the protein misfolding disorder. In some embodiments, the metabolic pathway is associated directly or indirectly with at least one of, synthesis, formation, stability, levels, activity and function of the metabolite. It should be understood that the manipulations in the metabolic pathway in the yeast cell/s, cell lines, yeast cell population, and/or any progeny thereof, may be genetic manipulations and/or modifications, epigenetic manipulations or any combinations thereof. In some embodiments, manipulation as indicated herein refers to genetic and/or epigenetic manipulation. More specifically, manipulation and/or modification refers to causing at least one of mutation, alteration, abolishment and variation in at least one gene encoding at least one protein that participate in at least one metabolic pathway involved directly or indirectly with at least one of, synthesis, formation, stability, levels, activity and function of the metabolite. In some embodiments, the genetic and/or epigenetic manipulation is performed using gene editing systems. The specific gene encode a protein that participates at least in part of the metabolic pathway. Introduction of such genetic manipulation disturbs the normal function of said protein, thereby leading to accumulation of the specific metabolite. The systems and methods of the invention comprise yeast cell/s and/or cell line/s having at least one manipulation and/or modification, for example, genetic and/or epigenetic manipulation or modification in at least one yeast metabolic pathway. A metabolic pathway, as used herein is a linked series of chemical reactions occurring within a cell. The reactants, products, and intermediates of an enzymatic reaction are known as metabolites, which are modified by a sequence of chemical reactions catalyzed by enzymes. In most cases of a metabolic pathway, the product of one enzyme acts as the substrate for the next. Different metabolic pathway/s function based on the location within a eukaryotic cell and the significance of the pathway in the given compartment of the cell. For example, the mitochondrial membrane or alternatively, the cytosol. There are two types of metabolic pathways that are characterized by their ability to either synthesize molecules with the utilization of energy (anabolic pathway) or break down of complex molecules by releasing energy in the process (catabolic pathway). In addition to the two distinct metabolic pathways is the amphibolic pathway, which can be either catabolic or anabolic based on the need for or the availability of energy. A manipulation in at least one metabolic pathway in accordance with the invention encompasses a genetic modification that modulates (enhance or inhibit the expression, stability and/or activity) any of the enzymes participating in the pathway, specifically at any stage of the pathway thereby leading to the accumulation of the specific metabolite that shown by the present disclosure as participating in aggregation of pathogenic proteins (e.g. amyloid beta) associated with the protein misfolding disorder. The modification according to the invention may be at any metabolic pathway, either catabolic, anabolic or amphibolic pathway as discussed herein.

As indicated herein, the manipulation in at least one metabolic pathway of the yeast leads to accumulation of the specific metabolite shown by the present disclosure as participating in aggregation of pathogenic proteins associated or linked with the proteinopathy and/or protein misfolding disorder. Accumulation as used herein refers to addition, increase, multiplication, conglomeration, growth by addition, gathering, collecting, agglomeration, accession, intensification, multiplication, enlargement, augmentation in the amount, mass, concentration and/or quantity of the metabolite in the cell over time. The accumulation of the metabolite as referred to herein encompass any increase in about 5%, 10%, 15%, 20%, 25%, 30%, 35%, 40%, 45%, 50%, 55%, 60%, 65%, 70%, 75%, 80%, 85%, 90%, 95%, 100%, as compared to natural concentration, amount or quantity of the metabolite in the cell, under conditions were the metabolic pathway is not manipulated. In some embodiments, the amount, concentration and/or quantity of the accumulated metabolite may be increased by 2, 3, 4, 5, 6, 7, 8, 9, 10, 50, 100, 1000, 10,000, folds or more as compared with the amount, concentration and/or quantity of the metabolite in native condition of non-manipulated pathway.

The genetic manipulation discussed herein include any mutation, rearrangement, insertion, deletion, or substitution of one or more nucleotide/s in the coding and/or non-coding region/s of at least one gene that encodes at least one product involved directly or indirectly with at least one of, synthesis, formation, stability, levels, activity and function of the metabolite associated with the protein misfolding disorder.

The term “mutation” as herein defined refers to a change in the nucleotide sequence of the genome of an organism, specifically, yeast cell/s or cell line/s. Mutations may or may not produce observable (phenotypic) changes in the characteristics of an organism. Mutation can result in several different types of change in the DNA sequence. These changes may have no effect, alter the product of a gene, or prevent the gene from functioning properly or completely. There are generally three types of mutations, namely single base substitutions, rearrangement, insertions and deletions and mutations defined as “chromosomal mutations”.

The term “single base substitutions” as herein defined refers to a single nucleotide base which is replaced by another. These single base changes are also called point mutations. There are two types of base substitutions, namely, “transition” and “transversion”. When a purine base (i.e., Adenosine or Thymine) replaces a purine base or a pyrimidine base (Cytosine, Guanine) replaces a pyrimidine base, the base substitution mutation is termed a “transition”. When a purine base replaces a pyrimidine base or vice-versa, the base substitution is called a “transversion”.

Single base substitutions may be further classified according to their effect on the genome, as follows: In missense mutations the new base alters a codon, resulting in a different amino acid being incorporated into the protein chain. In nonsense mutations the new base changes a codon that specified an amino acid into one of the stop codons (taa, tag, tga). This will cause translation of the mRNA to stop prematurely and a truncated protein to be produced. This truncated protein will be unlikely to function correctly.

In silent mutations no change in the final protein product occurs and thus the mutation can only be detected by sequencing the gene. Most amino acids that make up a protein are encoded by several different codons (see genetic code). So, if for example, the third base in the ‘cag’ codon is changed to an ‘a’ to give ‘caa’, a glutamine (Q) would still be incorporated into the protein product, because the mutated codon still codes for the same amino acid. These types of mutations are‘silent’ and have no detrimental effect. Mutation may also arise from insertions of nucleic acids into the DNA or from duplication or deletions of nucleic acids therefrom. As herein defined, the term “insertions and deletions” refers to extra base pairs that are added or deleted from the DNA of a gene, respectively. The number of bases can range from a few to thousands. More specifically, 1 base or more, 2, 3, 4, 5, 6, 7, 8, 9, 10, 15, 20, 25, 30, 35, 40, 45, 50, 55, 60, 65, 70, 75, 80, 85, 90, 95, 100, 150, 200, 250, 300, 350, 400, 450, 500, 550, 600, 650, 700, 750, 800, 850, 900, 950, 1000, 2000, 3000, 4000, 5000, 6000, 7000, 8000, 9000, 10000 and more, 50,000 and more. It should be noted that deletion in the gene, specifically, at least one gene encoding a product that participate in at least one metabolic pathway, in accordance with the invention may be a partial or complete deletion of specific intron/s, exon/s or the entire gene or any homolog thereof, optionally, in both alleles. Insertions and deletions of one or two bases or multiples of one or two bases cause, inter alia, frame shift mutations (i.e., these mutations shift the reading frame of the gene). These can have devastating effects because the mRNA is translated in new groups of three nucleotides and the protein being produced may be useless. Insertions and deletions of three or multiples of three bases may be less substantial because they preserve the open reading frame. It should be understood that the yeast cell and/or yeast cell line and/or yeast cell population, and/or any progeny thereof in accordance with the systems and method of the present invention may carry any one of the above mutations or genetic modifications or any combinations thereof, in one (in haploid and diploid yeasts) or in both alleles (in diploid yeasts). Still further, in some embodiments the yeast cell/s of the systems and method of the present invention undergo alternatively or additionally at least one epigenetic manipulation in at least one nucleic acid sequence that encodes or regulates the expression of a product involved directly or indirectly in the metabolic pathway. Such epigenetic manipulation (e.g., methylation, gene repression) leads to accumulation of at least one metabolite shown by the present disclosure as participating in aggregation of pathogenic protein/s associated with the protein misfolding disorder. The term “epigenetic modification” refers to a change in genetic information that does not arise from a change in a nucleotide sequence (e.g., a DNA sequence). Typically, epigenetic modifications affect the expression or activity of a target chromatin site (e.g., the expression or activity of a gene), although an epigenetic modification can be any modification of genetic material that does not arise from a nucleotide sequence change but produces a change in a phenotype. Epigenetic modifications typically comprise modifications to a nucleic acid (e.g., DNA) or a protein (e.g., a histone). Such modifications typically comprise methylation, dimethylation, trim ethylation, demethylation, acetylation, deacetylation, citrullination, or a combination thereof. Epigenetic modifications can either decrease or increase the expression or activity of a target site (e.g., gene expression or activity of a gene encoding a protein participating in the metabolic pathway). Epigenetic modifications, and the resulting effects (e.g., changes in gene expression or phenotype), can be either transient or persistent. It should be therefore understood that the manipulated yeast cells of the systems and methods of the invention may comprise or exposed to any reagent or means for epigenetic manipulation or modification in at least one metabolic pathway that leads to accumulation of the specific metabolite associated with the protein misfolding disorder. Such manipulation may be performed using any silencing means, for example, specific siRNAs, or other inhibitory nucleic acid molecules, or any gene editing systems that may lead to manipulation (e.g., the CRISPRi or CRISPRa systems) or any chimeras or fusion proteins thereof, for example, dCAS-methyltransferase directed (by specific gRNAs) at regulatory or non-regulatory sequences of a gene encoding a product participating directly or indirectly in a metabolic pathway associated with the metabolite, thereby leading to accumulation thereof.

It should be also appreciated that the genetic and/or epigenetic manipulation may be transient, stable and/or inducible.

In some specific embodiments, the manipulated yeast cell/s provided by the systems and methods of the invention may carry at least one manipulation that affects a yeast metabolic pathway. In more specific embodiments, such genetic and/or epigenetic manipulation results in reduced function of at least one gene product that participates in the specific target metabolic pathway. Therefore, in some embodiments, the genetic and/or epigenetic manipulation performed in the yeast cells of the systems and methods of the invention leads to loss of function. More specifically. “Loss of function” generally refers to reduction of function or absence of function as compared with a reference level. The reference level may be, e.g., a normal or average level of function possessed by a normal gene product or found in a healthy cell or subject. In certain embodiments the reference level may be the lower limit of a reference range. In certain embodiments the function may be reduced by at least about 5%, 10%, 15%, 20%, 25%, 30%, 35%, 40%, 45%, 50%, 55%, 60%, 65%, 70%, 75%, 80%, 85%, 90%, 95%, 99%, or 100% of the reference level.

In some embodiments, the yeast cell/s and/or cell line/s of the systems and methods disclosed herein carry at least one modification or were manipulated or modified (genetically and/or epigenetically) to loos the function, either by disruption and/or knock out of the target gene that encodes a product participating in at least one metabolic pathway. This manipulation causes the accumulation of the metabolite in the modified and/or manipulated cell that lost the function of the modified and/or manipulated gene. Still further, a “loss of function mutation” in a gene refers to a mutation that causes loss (reduction or absence) of at least one function normally provided by a gene product of the gene. A loss of function mutation or modification in a gene or group of genes, for example, a native yeast gene (e.g., the CYS4 gene) that encodes a product that participates in a native yeast metabolic pathway, may result in a reduced total level of a gene product of the gene in a cell that carry the mutation (e.g., due to reduced expression of the gene/s, reduced stability of the gene/s product/s, or both), reduced or altered activity or function per molecule of the gene product encoded by the mutant or modified gene, or both. The reduction in expression, level, activity per molecule, or total function may be partial or complete. A mutation or modification that confers a complete loss of function, or an allele harboring such a mutation or modification, may be referred to as a null mutation or null allele, respectively. In some embodiments a loss of function mutation or modification in a gene results in a reduction of at least 10%, 20%, 30%, 40%, 50%, 60%, 70%, 80%, 90%, 95%, 99%, or 100% in the level or activity of a gene product of the mutant gene, as compared with level or activity of a gene product encoded by a normal allele of the gene. A loss of function mutation may be an insertion, deletion, rearrangement or point mutation or modification. For example, a point mutation may introduce a premature stop codon, resulting in a truncated version of the normal gene product that lacks at least a portion of a domain that contributes to or is essential for activity, such as a catalytic domain or binding domain, or may alter an amino acid that contributes to or is essential for activity, such as a catalytic residue, site of post-translational modification, etc. Alternatively, the genetic manipulation may occur in any coding or non-coding regulatory region that affects the stability, expression and splicing thereof. Still further, as indicated above, the loss of function manipulations or modifications may also involve epigenetic manipulations. It should be however noted that in some alternative embodiments, the manipulated yeast cells of the invention may be manipulated to express either endogenously (by enhancing transcription and/or translation or reducing repression of transcription and/or translation) or exogenously (e.g., using an exogenously added nucleic acid sequence, optionally provided in a vector such as a plasmid) metabolic pathway or any parts thereof, that leads to accumulation of the specific metabolite. Such manipulated and/or modified cells may therefore carry a gain of function mutation or genetic and/or epigenetic manipulation or modification. In some embodiments, the yeast cell/s and/or cell line/s of the systems and methods disclosed herein carry at least one modification or were manipulated or modified (genetically and/or epigenetically) to gain the function, either by exogenously expressing and/or enhancing the expression and/or stability of a target gene that encodes a product participating in at least one metabolic pathway, and/or is associated with a pathogenic protein accumulated in proteinopathies. This manipulation causes the accumulation of the metabolite in the modified and/or manipulated cell that gain the function of the modified and/or manipulated gene. In more specific embodiments, a “gain of function mutation” in a gene refers to a mutation that causes gain (increase or presence) of at least one function normally not provided. A gain of function mutation in a gene, for example, a native yeast gene that encodes a product that participates in a native yeast metabolic pathway, may result in enhanced total level of a gene product of the gene in a cell that carry the mutation (e.g., due to enhanced expression of the gene, enhanced stability of the gene product, or both), enhanced activity per molecule of the gene product encoded by the mutant gene, or both. The increase in expression, level, activity per molecule, or total function may be partial or complete. Alternatively, the cells may express exogenously added gene that encodes the desired product that lead to accumulation of the metabolite. In some embodiments a gain of function mutation in at least one gene may result in an increase of at least 5%, 10%, 20%, 30%, 40%, 50%, 60%, 70%, 80%, 90%, 95%, 99%, or 100% in the level or activity of a gene product of the mutant gene, as compared with level or activity of a gene product encoded by a normal allele of the gene. It should be understood that in some embodiments, the genetic and/or epigenetic manipulation of the yeast cell and/or yeast cell line, and/or yeast cell population, and/or any progeny thereof in accordance with the invention may be performed using any editing system. Non-limiting r examples include, but are not limited to. Transcription activator-like effector nucleases (TALEN), Zinc-finger nucleases (ZFNs), clustered regularly interspaced short palindromic repeats (CRISPR-Cas) system, or any fusion proteins thereof. In yet some alternative or additional embodiments, the CRISPR system may be used for epigenetic manipulations, for example using a mutated Cas (dCAS) protein devoid of nucleolytic activity, fused to an effector molecule such as methyl transferase that methylates the target sequences targeted by the gRNAs, or a dCAS protein fused to a repressor, specifically. CRISPR interference (CRISPRi) (e.g., dCAS-repressor) or alternatively, dCAS fused to activator, specifically, CRISPR activation (CRISPRa) (e.g., dCAS-activator). A non-limiting example for repressor useful in the invention as the effector/modifier component, may be the Krüppel associated box (KRAB) domain, which enhances repression of the targets. A non-limiting example for such activator may be the Herpes simplex virus protein vmw65, also known as VP16.

As indicated above, in some alternative embodiments, the system of the invention may further comprise as a second element or component, at least one of reagents and/or means for detecting or determining at least one phenotype associated with the accumulation of the metabolite. “Means” as used herein refers to any cellular and non-cellular means, specifically, any natural or artificial cell or cell parts, organs, tissues or any equipment, facility, instrument, machine, program, required for determination, quantitation, recording, computing, visualizing, and evaluating the accumulation of the metabolite shown by the present disclosure as participating in aggregation of pathogenic protein/s involved in the protein misfolding disorder.

In some embodiments, the system disclosed herein further comprises at least one validation means for the candidate therapeutic compound. In some embodiments, the validation means is at least one of: (a), at least one unicellular organism that display accumulation of the metabolite and/or accumulation of the pathological protein; (b), at least one multicellular eukaryotic organism that display accumulation of the metabolite and/or accumulation of said pathological protein; and (c), at least one mammalian cell that display accumulation of the metabolite and/or accumulation of said pathological protein; and (d), at least one mammalian animal model that display accumulation of the metabolite and/or accumulation of the pathological protein.

In yet some specific embodiments, the system/s and method/s provided by the invention may further comprise and use, at least one validation means for the candidate therapeutic compound. Validation means in accordance with the invention is used for checking, verifying, proving the validity or effectivity, establishing documentary evidence demonstrating that the identified compound is suitable for therapy. As indicated above, in more specific embodiments such validation means may be at least one of: (a) at least one unicellular organism that display accumulation of the metabolite; (b) at least one multicellular eukaryotic organism that display accumulation of the metabolite; (c) at least one mammalian cell that display accumulation of the metabolite; and (d) at least one mammalian animal model that display accumulation of the metabolite. It should be understood that the unicellular or multicellular organism used as a validation mean may be any unicellular and/or multicellular organism used as a model for the specific proteinopathy.

Mores specifically, in some embodiments, as discussed in (a), the system of the invention may include as a means for evaluation, any unicellular organism, specifically, any eukaryotic or prokaryotic cell that display accumulation of the metabolite.

In some embodiments, any eukaryotic cells, either of a unicellular or of a multicellular eukaryotic organism or prokaryotic cells (bacteria or archaea) may be used as an evaluation means by the systems of the invention. In some embodiments, eukaryotic cells and/or eukaryotic unicellular or multicellular organisms in accordance with the invention may include any eukaryotic cell or organism, for example, of any organism of the biological kingdom Animalia. In more specific embodiments, the eukaryotic cells of the invention may originate from a mammal, specifically, a human. In yet some further embodiments, such mammal may include any member of the mammalian nineteen orders, specifically, Order Artiodactyla (even-toed hoofed animals), Order Carnivora (meat-eaters), Order Cetacea (whbales and purpoises), Order Chiroptera (bats), Order Dermoptera (colugos or flying lemurs), Order Edentata (toothless mammals), Order Hyracoidae (hyraxes, dassies), Order Insectivora (insect-eaters), Order Lagomorpha (pikas, hares, and rabbits), Order Marsupialia (pouched animals), Order Monotremata (egg-laying mammals), Order Perissodactyla (odd-toed hoofed animals), Order Pholidata, Order Pinnipedia (seals and walruses), Order Primates (primates), Order Proboscidea (elephants), Order Rodentia (gnawing mammals), Order Sirenia (dugongs and manatees), Order Tubulidentata (aardvarks). In some specific embodiment, such mammal may be at least one of a Cattle, domestic pig (swine, hog), sheep, horse, goat, alpaca, lama and Camels. In yet some further embodiments, the eukaryotic cells that may be used by the systems and methods disclosed herein, may be cells that originate from rodent, as it represents the most popular and commonly accepted animal model in research.

In yet some further specific embodiments, additional yeast cell lines may be used for evaluation. In some specific embodiments, Schizosaccharomyces pombe or Candida albicans may be used as a validation means by the systems of the invention.

Thus, in some embodiments, Schizosaccharomyces pombe may be used as a validation means by the systems and methods of the invention. Schizosaccharomyces pombe, also called “fission yeast”, is a species of yeast used in traditional brewing and as a model organism in molecular and cell biology. It is a unicellular eukaryote, whose cells are rod-shaped. Cells typically measure 3 to 4 micrometers in diameter and 7 to 14 micrometers in length. Its genome, which is approximately 14.1 million base pairs, is estimated to contain 4,970 protein-coding genes and at least 450 non-coding RNAs.

These cells maintain their shape by growing exclusively through the cell tips and divide by medial fission to produce two daughter cells of equal size, which makes them a powerful tool in cell cycle research.

In yet some further embodiments, Candida albicans may be used as a validation means by the systems and methods of the invention. Candida albicans is an opportunistic pathogenic yeast that is a common member of the human gut flora. It does not proliferate outside the human body. It is detected in the gastrointestinal tract and mouth in 40-60% of healthy adults. It is usually a commensal organism, but can become pathogenic in immunocompromised individuals under a variety of conditions. It is one of the few species of the genus Candida that causes the human infection candidiasis, which results from an overgrowth of the fungus. C. albicans is the most common fungal species isolated from biofilms either formed on (permanent) implanted medical devices or on human tissue.

In some further specific and non-limiting embodiments, the yeast cells applicable in the systems of the invention may be the W303-derivative K6001, that is a key model organism for research into aging. In more specific embodiments, this strain may be also referred to as MATa/MATα {leu2-3,112 trp1-1 can1-100 ura3-1 ade2-1 his3-11,15} [phi⁺]. It should be appreciated that such additional yeast may be used also by any of the methods of the invention as described herein after.

In yet some further specific embodiments, mammalian cells may be used as an evaluation means by the systems of the invention. More specifically, the mammalian cell may be any cell model, for example, primary cells, including Neural stem cells, lymphoblast and fibroblast, hematopoietic stem cells (HSCs). Mesenchymal stem cells, Muscle stem cells, T cells, embryonic stem cell (ESC)-derived and Induced Pluripotent Stem (iPS) cells derived from a patient suffering from said protein misfolding disorder and cell lines. Specifically, embryonic stem cells, or human embryonic stem cells (hESCs), that were obtained from self-umbilical cord blood just after birth. Embryonic stem cells are pluripotent stem cells derived from the early embryo that are characterized by the ability to proliferate over prolonged periods of culture while remaining undifferentiated and maintaining a stable karyotype, with the potential to differentiate into derivatives of all three germ layers, hESCs may be also derived from the inner cell mass (ICM) of the blastocyst stage (100-200 cells) of embryos generated by in vitro fertilization. However, methods have been developed to derive hESCs from the late morula stage (30-40 cells) and, recently, from arrested embryos (16-24 cells incapable of further development) and single blastomeres isolated from 8-cell embryos. In some specific and non-limiting embodiments, neuronal cell/s or cell line/s may be also used, as also shown by the present Examples), as a validation mean in the systems and methods of the present disclosure.

In further embodiments, the eukaryotic cells according to the invention are totipotent stem cells. Totipotent stem cells are versatile stem cells and have the potential to give rise to any and all human cells, such as brain, liver, blood or heart cells or to an entire functional organism (e.g., the cell resulting from a fertilized egg). The first few cell divisions in embryonic development produce more totipotent cells. After four days of embryonic cell division, the cells begin to specialize into pluripotent stem cells. Embryonic stem cells may also be referred to as totipotent stem cells.

In further embodiments, the eukaryotic cells according to the invention are pluripotent stem cells. Similar to totipotent stem cells, a pluripotent stem cell refers to a stem cell that has the potential to differentiate into any of the three germ layers: endoderm (interior stomach lining, gastrointestinal tract, the lungs), mesoderm (muscle, bone, blood, urogenital), or ectoderm (epidermal tissues and nervous system). Pluripotent stem cells can give rise to any fetal or adult cell type. However, unlike totipotent stem cells, they cannot give rise to an entire organism. On the fourth day of development, the embryo forms into two layers, an outer layer which will become the placenta, and an inner mass which will form the tissues of the developing human body. These inner cells are referred to as pluripotent cells.

In still further embodiments, the eukaryotic cells that may be applicable for the methods according to the invention, are multipotent progenitor cells. Multipotent progenitor cells have the potential to give rise to a limited number of lineages. As a non-limiting example, a multipotent progenitor stem cell may be a hematopoietic cell, which is a blood stem cell that can develop into several types of blood cells but cannot into other types of cells. Another example is the mesenchymal stem cell, which can differentiate into osteoblasts, chondrocytes, and adipocytes. Multipotent progenitor cells may be obtained by any method known to a person skilled in the art. More specifically, hematopoietic stem cells (HSCs) originate from the bone marrow. They first differentiate into multipotent progenitor (MPP) cells, and then may differentiate to common lymphoid progenitor (CLP) cells. Still further, in some embodiments, cells suitable in the present application may be immobilized HSC. Hematopoietic stem cells (HSCs) normally reside in the bone marrow but can be forced into the blood, a process termed bone marrow mobilization used to harvest large numbers of HSCs in peripheral blood. One mobilizing agent of choice in accordance with the invention may be granulocyte colony-stimulating factor (G-CSF). The resulting HSCs are further referred as mobilized HSCs.

In some embodiments, pluripotent cells may be used as an evaluating means by the systems and methods of the invention. It should be appreciated that in some embodiments, the target cells may be pluripotent cells, specifically, hematopoietic pluripotent cells. The term “pluripotent” refers to cells with the ability to give rise to progeny that can undergo differentiation, under the appropriate conditions, into cell types that collectively demonstrate characteristics associated with different cell lineages. In yet some further embodiments, such pluripotent cells may be induced pluripotent stem cell. As used herein, an induced pluripotent stem (iPS) cell is a cell that is derived from a somatic cell by reprogramming the cell to a pluripotent state. iPS cells possess certain key features of ES cells including cell morphology, colony morphology, long-term self-renewal, expression of pluripotency-associated markers, similar genome-wide expression profile, ability to form teratomas in immunocompromised mice, and ability to give rise to cells of multiple cell lineages in vitro under appropriate conditions. It will be understood that the term “iPS cell” includes the original derived pluripotent cell and its descendants that retain pluripotent stem cell properties. “Reprogramming”, as used herein, refers to altering the differentiation state or identity of a cell (e.g., its cell type) or a process by which this occurs. In general, reprogramming a first cell generates a second cell with a differentiation state or identity distinct from that which would result from a differentiation program that the first cell or a corresponding cell would normally follow in vivo and results in cells of one or more types distinct from those that the first cell or a corresponding cell would give rise to in vivo. A “corresponding cell” is a cell of the same type or having sufficiently similar features that a person of ordinary skill in the art would reasonably consider it to be of the same or substantially the same cell type. In some specific embodiments, several neuronal cell/s or cell line/s, or cells of a neuronal origin, may be used as a validation means in the systems and methods. Various cells and cell lines, specifically, cells derived from neuronal tissue, or of a neuronal source, may be applicable in the systems and methods of the present disclosure. These cells may include but are not limited to the following cell types: astrocytes, Neurons, Neuronal schwann cells, Brain endothelial cells and fibroblasts, glial cells, Pituitary tumor cells, retail cells, medulloblastoma cells, brain tumor stem cell lines, GBM brain tumor cell lines and oligenglioma. The cells may be of a primary source or not, and may be of any vertebrate source, specifically, mammalian source. More specifically, in some embodiments, astrocytes, may include PG-4 (S+L−) cells (Cat Brain Astrocyte), G355-5 cells (Cat Brain Astrocyte), SVG p12 cells (Human Brain Astroglia), C8-D30 cells (Mouse Brain/cerebellum Astrocyte), C8-S cells (Mouse Brain/cerebellum Astrocyte), C8-D1A cells (Mouse Brain/cerebellum Astrocyte), Swiss SFME cells (Mouse Embryo Astrocyte) BALB SFME cells (Mouse Embryo Astrocyte) C6 cells (Rat Brain Astrocyte) DI TNC1 cells (Rat Brain/diencephalon Astrocyte), CTX TNA2 cells (Rat Brain/cortex Astrocyte); Neurons cells useful in the present disclosure include, but are not limited to SK-N-AS cells (Human Brain; derived from metastatic site bone marrow Neuroblast), (SK-N-FI cells (Human Brain; derived from metastatic site: bone marrow Neuroblast), SK-N-DZ cells (Human Brain; derived from metastatic site: bone marrow Neuroblast), CRL-2266 SH-SY-5Y Human Brain; derived from metastatic site: bone marrow Neuroblast), M17 cells (Human Brain; derived from metastatic site: bone marrow Neuroblast), LUHMES cells (Human Mesencephalon/mid brain Dopaminergic neuron), HCN-2 cells (Human Brain Cortical neuron), neuro-2a cells (Mouse Brain Neuroblast), NB41A3 cells (Mouse Brain Neuroblast), NIE-115 cells (Mouse Brain Neuroblast) Cath.a cells (Mouse Brain Neuron), CRL-2925 NE-4C cells (Mouse Brain, neuroectodermal Neural stem cells), NE-GFP-4C cells (Mouse Brain, neuroectodermal Neural stem cells), QNR/D cells (Quail Neuroretina Retinal gangion/amacrine), QNR/K2 cells (Quail Neuroretina Retinal gangion/amacrine), PC-12 cells (Rat Adrenal gland Neuroblast), RN-33B cells (Rat Brain/medullary raphe nucleus Neuron Essential Neural Cell Lines). Still further, in some embodiments, Neuronal schwann cells applicable in the present disclosure include, but are not limited to sNF02.2 cells (Human Peripheral nervous system, derived from metastatic site: lung Neurofibromatosis type 1 (Nf1)), sNF94.3 cells (Human Peripheral nervous system, derived from metastatic site: lung Neurofibromatosis type 1 (Nf1)), SW10 cells (Mouse Nerve Normal), R3 cells (Rat Sciatic nerve Normal), RSC96 cells (Rat Nerve Normal). RT4-D6P2T cells (Rat Peripheral nervous system Schwannoma). S16 cells (Rat Sciatic nerve Normal), S42 cells (Rat Sciatic nerve Normal), S16Y cells (Rat Sciatic nerve Normal0. Still further, in some embodiments, Various Brain endothelial cells and fibroblasts may be also applicable in the present disclosure, these cells include HBEC-5i cells (Human Brain/cerebral cortex Cerebral microvascular endothelium), bEnd.3 cells (Mouse Endothelioma: cerebral cortex Endothelial), H19-7/IGF-IR cells (Rat Brain/hippocampus Fibroblast). In yet some further embodiments, various Glial cells may be applicable in the systems and methods of the present disclosure, for example, DBTRG-05MG cells (Human Glioblastoma Glial cell), M059K cells (Human Malignant glioblastoma: glioma Glial cell), M059J cells (Human Malignant glioblastoma; glioma Glial cell), A-172 cells (Human Glioblastoma Glial cell), SVG p12 cells (Human Brain Astroglia CRL-3304 HMC3 Human Brain Microglia), EOC 2 cells (Mouse Brain Microglia), EOC 13.31 cells (Mouse Normal Microglia), EOC 20 cells (Mouse Brain Microglia), SIM-A9 cells (Mouse Brain Microglia). C8-D30 cells (Mouse Brain/cerebellum Monocyte/macrophage microglia), C6 cells (Rat Glioma Glial cell), C6/lacZ cells (Rat Glioma Glial cell), 9L/lacZ cells (Rat Gliosarcoma Glial cell), C6/lacZ7 cells (Rat Glioma Glial cell), −2815 cells (HAPI Rat Brain Microglia), F98 cells (Rat Undifferentiated malignant glioma Glial cell), RG2 [D74] cells (Rat Differentiated malignant glioma Glial cell), F98npEGFRvIII cells (Rat Undifferentiated malignant glioma Glial cell) F98 EGFR cells (Rat Undifferentiated malignant glioma Glial cell, and PC12 is a rat cell line (derived from a pheochromocytoma of the adrenal medulla).

Still further, in some embodiments, as also demonstrated by the following examples, the SH-SY5Y cell line may be applicable as a validation means in the systems and methods of the present disclosure. More specifically, the human SH-SY5Y cell line was derived by subcloning from the parental metastatic bone tumor biopsy cell line SK-N-SH SH-SY5Y. The cells can grow continuously as undifferentiated cells that have a neuroblast-like morphology and express immature but not mature neuronal markers. Differentiated SH-SY5Y cells are morphologically similar to primary neurons with long processes and exhibit a decrease in proliferation rate, exit the cell cycle and enter Go, and show increased expression of neuron-specific markers.

In yet some further embodiments, multicellular organisms may be used as a validation means by the systems of the invention. In some embodiments, the models may be based on multicellular organisms that were genetically and/or epigenetically modified. Such modification may be performed using any gene editing system [e.g., TALEN, ZFNs, CRISPR-Cas, or any fusion proteins thereof, for example CRISPRi (e.g., dCAS-repressor, or dCas-methyl transferase) or alternatively, CRISPRa (e.g., dCAS-activator)] or alternatively, by any know gene silencing means (e.g., siRNA, anti-sense nucleic acids, miRNA and the like).

In more specific embodiments, multicellular eukaryotic organism useful as an evaluation means in the systems and methods of the invention may be Nematodes models (Caenorhabditis elegans using any gene silencing means, for example, RNAi, mutations or any gene editing system, for example, CRISPR-Cas and the like).

In some embodiments, the multicellular organism C. elegans may be used by the systems and methods of the invention as a means for evaluation. More specifically, Caenorhabditis elegans is a free-living, transparent nematode, about 1 mm in length, that lives in temperate soil environments. It is the type species of its genus. It was previously named Rhabditides elegans, and has been placed it in the genus Caenorhabditis. C. elegans is an unsegmented pseudocoelomate and lacks respiratory or circulatory systems. C. elegans is unsegmented, vermiform, and bilaterally symmetrical. It has a cuticle (a tough outer covering, as an exoskeleton), four main epidermal cords, and a fluid-filled pseudocoelom (body cavity). It also has some of the same organ systems as larger animals. About one in a thousand individuals is male and the rest are hermaphrodites. The basic anatomy of C. elegans includes a mouth, pharynx, intestine, gonad, and collagenous cuticle. Like all nematodes, they have neither a circulatory nor a respiratory system.

In yet some further embodiments, Drosophila melanogaster may be used as the multicellular eukaryotic organism applicable as a validation means by the systems and methods of the invention. Drosophila melanogaster is a species of fly (the taxonomic order Diptera) in the family Drosophilidae. The species is known generally as the common fruit fly (though inaccurately) or vinegar fly. The D. melanogaster is a specie widely used as a model organism, for biological research in genetics, physiology, microbial pathogenesis, and life history evolution. Drosophila is typically used in research because it can be readily reared in the laboratory, has only four pairs of chromosomes, breeds quickly, and lays many eggs. Still further, in some embodiments, zebrafish may be used as the multicellular eukaryotic organism applicable as a validation means by the systems and methods of the invention. More specifically, zebrafish (Danio rerio) is a freshwater fish belonging to the minnow family (Cyprinidae) of the order Cypriniformes. The zebrafish is a prominent vertebrate model system for comprehensive analysis of the unique functions of genes along with their signaling pathways during development and neurodegeneration. Zebrafish possesses several distinct advantages over other vertebrate models. Specifically, the simplicity of their natural habitat, short generation times of 3-5 months that enhances the rate of experimental progress. Still further, the zebrafish possess external fertilization, and their development pattern facilitates the observation and experimental manipulation of the embryos. Moreover, the unique advantages of the zebrafish are the unrivalled optical clarity of the embryos, allowing visualization of individual genes (fluorescently labelled or dyed) throughout the developmental process using non-invasive imaging techniques. Although zebrafish are different to mammals in organization structure of the central nervous system, several cerebral nuclei in the zebrafish brain including the basal ganglia, striatum, hippocampus and amygdala have high homogeneity to mammals. Thus, the zebrafish possess a vertebrate neural structural organization, and their genome has several gene orthologs similar to those mutated in human Familial Alzheimer's disease (FAD). Zebrafish models are progressively emerging and becoming a powerful tool for in vivo study of neurodegenerative disorders. Extensive use of zebrafish in pharmacology research or drug screening is due to the high conserved evolution and 87% homology to humans. For example, zebrafish models have been used successfully to simulate the pathology of Alzheimer's disease (AD) as well as Tauopathy. Their relatively simple nervous system and the optical transparency of the embryos permit real-time neurological imaging. Several zebrafish models have been established by placing Aβ central to the disease pathology to simulate AD. It was reported that higher levels of Aβ monomers can stimulate angiogenic sprouting in the developing zebrafish hind brain. Still further, it was recently shown that both APP and Aβ-deficient larvae displayed cerebrovascular defects. Besides the Aβ model, research has also focused on the generation of zebrafish models with Tauopathy. Cytoskeletal disruption occurred on expressing frontotemporal dementia with Parkinsonism linked to chromosome 17, a mutant form of human Tau in the neurons of the zebrafish, which resembled the neurofibrillary tangles observed in AD.

Although zebrafish are different to mammals in organization structure of the central nervous system, several cerebral nuclei in the zebrafish brain including the basal ganglia, striatum, hippocampus and amygdala have high homogeneity to mammals. Specifically, in neurodegenerative disorders, especially AD, PD, and ALS, zebrafish have been validated as a feasible tool for research use. Advances in novel imaging strategies, tractable behavioral tests and high-throughput drug screening methods in zebrafish, and more new drug targets have recently been discovered for the treatment of NDD. Zebrafish provide a useful platform for drug discovery in NDD that is time- and cost-efficient. Cerebroventricular microinjection of Aβ42 into zebrafish brain induces Aβ protein accumulation, apoptotic cell death, microglia activation and synaptic degeneration. Islet amyloid polypeptide (IAPP) or Aβ microinjection into zebrafish embryos can produce an AD-like animal model to study amyloidosis. The Swedish mutant APP is known to cause familial AD and zebrafish harbor two genes (appa and appb) that are similar to human APP. Although APP gene is evolutionary conserved in vertebrates, the function of appb is more paramount than appa since loss of appb not only impair locomotor behavior, motor neuron patterning and formation, Mauthner cell development but also disrupt cell adhesion during early development in zebrafish. Neurofibrillary tangles (NTFs) are another typical hallmark of AD and are composed of highly phosphorylated tau. Tau is a microtubule-associated protein which is involved in modulating microtubule assembly and stabilization. Hyperphosphorylated tau protein tends to form NTFs and affects microtubule normal function, microtubule dynamics and axonal transport. A stable Tg zebrafish model with mutations on tau encoding gene MAPT (microtubule-associated protein tau), such as Tau-P301L and Tau-A152T, has been established to mimic AD key pathological features of tauopathies. It should be understood that each of the disclosed zebrafish models is applicable as a validation means in the disclosed systems and methods. It should be understood that the invention encompasses the use of any in vivo animal model of any eukaryotic organism, specifically, any mammalian organism, and more specifically any rodent model. Rodents are mammals of the order Rodentia, which are characterized by a single pair of continuously growing incisors in each of the upper and lower jaws. Rodents are the largest group of mammals. Non-limiting examples for such rodents that are applicable in the present invention, appear in the following list of rodents, arranged alphabetically by suborder and family. Suborder Anomaluromorpha includes the anomalure family (Anomaluridae) [anomalure (genera Anomalurus, Idiurus, and Zenkerella)], the spring hare family (Pedetidae) [spring hare (Pedetes capensis)]. The suborder Castorimorpha includes the beaver family (Castoridae) [beaver (genus Castor), giant beaver (genus Castoroides: extinct)], the kangaroo mice and rats (family Heteromyidae) [kangaroo mouse (genus Microdipodops), kangaroo rat (genus Dipodomys), pocket mouse (several genera)], the pocket gopher family (Geomyidae) [pocket gopher (multiple genera)]. Suborder Hystricomorpha, includes the agouti family (Dasyproctidae), acouchy (genus Myoprocta) [agouti (genus Dasyprocta)], the American spiny rat family (Echimyidae), the American spiny rat (multiple genera), the blesmol family (Bathyergidae) [blesmol (multiple genera)], the cane rat family (Thryonomyidae) [cane rat (genus Thryonomys)], the cavy family (Caviidae) [capybara (Hydrochoerus hydrochaeris), guinea pig (Cavia porcellus) mara (genus Dolichotis)], the chinchilla family (Chinchillidae) [chinchilla (genus Chinchilla), viscacha (genera Lagidium and Lagostomus)], the chinchilla rat family (Abrocomidae) [chinchilla rat (genera Cuscomys and Abrocoma)], the dassie rat family (Petromuridae) [dassie rat (Petromus typicus)], the degu family (Octodontidae) [degu (genus Octodon)], the diatomyid family (Diatomyidae), the giant hutia family (Heptaxodontidae), the gundi family (Ctenodactylidae) [gundi (multiple genera)], the hutia family (Capromyidae) [hutia (multiple genera)], the New World porcupine family (Erethizontidae) [New World porcupine (multiple genera)], the nutria family (Myocastoridae) [nutria (Myocastor coypus)], the Old World porcupine family (Hystricidae) [Old World porcupine (genera Atherurus, Hystrix, and Trichys)], the paca family (Cuniculidae) [paca (genus Cuniculus)], the pacarana family (Dinonmidae) [pacarana (Dinomys branickii)], the tuco-tuco family (Ctenomyidae) [tuco-tuco (genus Ctenomys)]. The suborder Myomorpha that includes the cricetid family (Cricetidae) [American harvest mouse (genus Reithrodonlomys), cotton rat (genus Sigmodon), deer mouse (genus Peromyscus), grasshopper mouse (genus Onychomys), hamster (various genera), golden hamster (Mesocricetus auratus), lemming (various genera) maned rat (Lophiomys imhausi), muskrat (genera Neofiber and Ondatra), rice rat (genus Oryzomys), vole (various genera), meadow vole (genus Microtus), woodland vole (Microtus pinetorum), water rat (various genera), woodrat (genus Neotoma), dipodid family (Dipodidae), birch mouse (genus Sicista), jerboa (various genera), jumping mouse (genera Eozapus, Napaeozapus, and Zapus)], the mouselike hamster family (Calomyscidae), the murid family (Muridae) [African spiny mouse (genus Acomys), bandicoot rat (genera Bandicota and Nesokia), cloud rat (genera Phloeomys and Crateromys), gerbil (multiple genera), sand rat (genus Psammomys), mouse (genus Mus), house mouse (Mus musculus), Old World harvest mouse (genus Micromys), Old World rat (genus Rattus), shrew rat (various genera), water rat (genera Hydromys, Crossomys, and Colomys), wood mouse (genus Apodemus)], thenesomvid family (Nesomyidae). African pouched rat (genera Beamys, Cricetomys, and Saccostomus)], the Oriental dormouse family (Platacanthomyidae) [Asian tree mouse (genera Platacanthomys and Typhlomys)], the spalacid family (Spalacidae) [bamboo rat (genera Rhizomys and Cannomys), blind mole rat (genera Nannospalax and Spalax), zokor (genus Myospalax), suborder Sciuromorpha], the dormouse family (Gliridae) [dormouse (various genera), desert dormouse (Selevinia betpakdalaensis)], the mountain beaver family (Aplodontiidae) [mountain beaver (Aplodontia rufa)], the squirrel family (Sciuridae) [chipmunk (genus Tamias), flying squirrel (multiple genera), ground squirrel (multiple genera), suslik (genus Spermophilus), marmot (genus Marmota), groundhog (Marmota monax), prairie dog (genus Cynomys), tree squirrel (multiple genera)]. In yet some further embodiments, the rodent model of the invention may be a mouse model. A mouse, plural mice, is a small rodent characteristically having a pointed snout, small rounded ears, a body-length scaly tail and a high breeding rate. The best known mouse species is the common house mouse (Mus musculus). Species of mice are mostly found in Rodentia, and are present throughout the order. Typical mice are found in the genus Mus. Several animal models, using mice and rats mainly, have been used to create genetically altered phenocopies of human AD. Transgenic mice overproducing mutant tau and APP proteins (e.g., PDAPP and PS19 mice) and/or some of the enzymes implicated in their metabolic processing have been bred. It should be understood that each one the disclosed animal models may be suitable as a validation means in the systems and methods of the present disclosure, and therefore is encompassed herein. In some embodiments, the transgenic mouse Tg2576, which overexpresses a mutant form of APP (695) associated with the Swedish mutation (K670N, M671NL), may be used herein as an animal model for AD. In yet some further embodiments, the triple-transgenic mouse model (3×Tg) that expresses three significant genes associated with familial AD (APPSwe, PSN1M146V, and tauP301L) may be applicable in the present disclosure. The 5×FAD mouse is an experimental model designed to reduce the time before amyloid plaques are formed. This transgenic mouse combines five mutations. Swedish mutation (APP KM670/671NL), London (V717l), Florida (APP I716V), L286V in PSN1, and M146L. It should be understood that in some embodiments, this animal model is also applicable in the present disclosure. Still further, the PS19 mice expressing the P301S mutant form of human microtubule-associated protein Tau(MAPT), is also an AD mouse model that may be applicable in accordance with some embodiments of the present disclosure. In yet some further embodiments, the E4FAD and E3FAD mouse models, which are crosses between the 5×FAD mice and mice expressing APOE4 and APOE3 human isoforms, may be applicable in some embodiments, as a model for sporadic AD.

Another interesting experimental tool that may be applicable in the methods and systems of the present disclosure is the ICV and intrahippocampal injections of Aβ peptides. Still further, in some embodiments, the ICV administration of soluble Aβ oligomers (AβOs), may b also applicable. These are potent neurotoxins derived from Aβ₁₋₄₂, which can be found in AD brains. In some embodiments of the system disclosed herein, the phenotype associated with accumulation of the metabolite and/or misfolding of the pathological protein is at least one of cell toxicity and formation of metabolite aggregates and/or aggregates of misfolded pathologic protein. As indicated herein above, the systems of the invention may comprise in some embodiments, means for detecting and determining the accumulation of the metabolite, as well as any phenotype associated with such accumulation. In some further embodiments, phenotype associated with accumulation of the metabolite as determined by the system of the invention may be at least one of cell toxicity and formation of metabolite aggregates, and/or aggregation or accumulation of pathologic proteins associated with proteinopathies. A phenotype, as used herein, is the composite of the observable characteristics or traits, of a cell that display accumulation of the specific metabolite. It includes morphological or physical and structural properties, as well as biochemical and physiological properties. As used herein, “phenotypes associated with accumulation of a specific metabolite” may be in some embodiments, cellular phenotypes that are associated with the protein misfolding disease and/or proteinopathy, that are detectable in cells of a subject suffering from the protein misfolding disorder. A cellular phenotype may be any detectable characteristic or property of a cell. In the context of the present disclosure, a cellular “phenotype” associated with a protein misfolding disease may be any detectable deviation from a characteristic or property displayed by a cell that distinguishes the cell from a normal cell or cell derived from a subject who does not have the protein misfolding disease and is not at increased risk of developing the protein misfolding disease relative to the general population.

Still further, in some embodiments, the toxicity is determined by measuring at least one of cell viability, cell proliferation, cell apoptosis, and any toxic phenotype on the organism or cell.

Thus, in some embodiments, a phenotype associated with accumulation of a specific metabolite may be cell toxicity. More specifically, toxicity or cell toxicity as used herein may be reflected by viability of the cells, shape, cell growth, cell function and cell death.

In yet some further embodiments, cell toxicity, may be reflected by induction of any one of oxidative stressors, nitrosative stressors, proteasome inhibitors, inhibitors of mitochondrial function, ionophores, inhibitors of vacuolar ATPases, inducers of endoplasmic reticulum (ER) stress, and inhibitors of endoplasmic reticulum associated degradation (ERAD). Thus, according to some embodiments, the system/s and methods of the invention may further comprise at least one reagent and/or means for measuring and/or detecting cell toxicity.

In some specific embodiments, toxicity, or cell toxicity, may be determined by the systems and/or methods of the invention by any means for quantification or measuring at least one of cell viability, cell proliferation, cell apoptosis, and any toxic phenotype on the organism or cell.

Thus, according to some embodiments, the system of the invention may further comprise at least one reagent and/or means for measuring at least one of cell viability, cell proliferation and cell apoptosis.

In some specific embodiments, cell viability may be determined by 2,3-bis-(2-methoxy-4-nitro-5-sulphophenyl)-2H-tetrazolium-5-carboxanilide (XTT) viability assay (not useful for cysteine accumulation), Methylene Blue, PrestoBlue viability reagent, the fluorescent intercalator 7-aminoactinomycin D (7-AAD). LIVE/DEAD Viability Kits, cell growth by turbidity, for example at OD600, or by any means for cell counting. Thus, in some embodiments, the systems of the invention may comprise at least one reagent required for performing any cell viability and proliferation assay, specifically, any of the assays disclosed above.

In yet some further embodiments, toxicity may be evaluated by measuring apoptosis of the cells. In some embodiments, apoptosis may be determined by at least one of DNA fragmentation (TUNNEL, terminal deoxynucleotidyl transferase dUTP nick end labeling), caspase and/or PARP1 phosphorylation, annexin V and propidium iodide (PI) assay. Thus, in some embodiments, the systems of the invention may comprise at least one reagent required for performing any apoptosis or any other cell death assay, specifically, any of the assays disclosed above.

It should be noted that in some embodiments, other toxic phenotype that may be determined on the organism/cell may include, activation of cell stress pathways (e.g., heat shock response), poor fertility, destruction of organs and tissue damages, DNA mutagenesis, ER stress, cell energy status and ATP content, oxidative stress, mitochondrial dysfunction, mitochondrial damage, activation of autophagy and activation of necrosis.

Thus, in some embodiments, the systems of the invention may comprise at least one reagent required for performing any of the cell toxicity assay disclosed above.

In some embodiments of the system of the present disclosure, at least one of, the accumulation of the metabolite and formation of metabolite aggregates and/or accumulation, and/or aggregation of the pathological protein is determined by at least one of metabolic profiling, microscopy, light diffraction, absorption or scattering assay, spectrometric assay, immunological assay, flow cytometry, liquid chromatography, nuclear magnetic resonance (NMR) and stereoscopy.

In yet some further embodiments, a phenotype associated with the accumulation of a specific metabolite may be the formation of metabolite aggregates. The term “metabolite aggregates” as used herein relates to accumulation of the specific metabolite in the cell in a specific fibrillar structures. More specifically, as used herein, Metabolite aggregates or metabolite structures refer to well-ordered assembly or elongated nanoscale fibrillary structures of non-protein entities such as metabolites. These supramolecular fibrillar structures are formed via self-association of entities that accumulate in a cell. The formation of these fibrils may resemble to amyloid-like fibrils and is typically described by a nucleation-dependent polymerization mechanism, which comprises nucleation and elongation, and is often considered as a kind of crystallization. The time period showing the mass increment of metabolite fibrils is referred to as the elongation phase, and the induction period before the elongation is called the lag phase. It should be noted that in some embodiments, metabolite aggregates may be also referred to herein as amyloid-like structures.

Thus, according to some embodiments, the system of the invention may further comprise at least one reagent and/or means for measuring formation and existence of metabolite aggregates. It should be however noted that in some embodiments, the system of the invention may comprise means and/or reagents for measuring and evaluating the level of the specific metabolite in the cell, specifically, reagents and means for detecting and quantifying metabolite accumulation in the cell.

In yet some further embodiments, accumulation of the metabolite and/or formation of metabolite aggregates may be determined by the system of the invention using at least one of metabolic profiling, microscopy, light diffraction, absorption or scattering assay, spectrometric assay, immunological assay, Nuclear magnetic resonance (NMR), Liquid Chromatography, flow cytometry, and stereoscopy.

In yet some further embodiments, metabolite aggregation may be measured using at least one of Dye-binding specificity (for example, using thioflavin T (ThT) and congo red, or staining with Proteostat) microscopy, X-ray fiber diffraction, X-ray crystallography. X-ray powder diffraction, X-ray single crystal diffraction, mass spectrometry (including Ion-mobility spectrometry, mass spectrometry (IMS-MS)), immunological assay (e.g. using a specific antibody that specifically recognize fibrillary assemblies), flow cytometry, circular dichroism (CD) spectrometry, vibrational CD, Raman Spectroscopy, density functional theory (DFT) quantum mechanics methods, Fourier-transformed infrared spectroscopy dynamic light scattering (DLS), liquid chromatography and NMR.

Microscopy, such as TEM (transmission electron microscope), confocal fluorescence microscopy, confocal Raman microscopy, indirect immunofluorescence. It should be understood that the systems and methods of the invention further encompass any reagent or means, for example, cellular or non-cellular, natural or artificial means, specifically, any equipment, machine, instrument, database, computer program, for performing any of the procedures and processes indicated above for detecting, determining measuring and/or visualizing the formation of metabolite aggregates or metabolite accumulation and/or accumulation of pathologic protein associated with proteinopathies and/or protein misfolding disorder. It should be appreciated that any means involved in the detection methods disclosed herein may be comprised within the system of the invention and used by any of the methods of the invention as a means for determining the accumulation and/or aggregation of the metabolite.

More specifically. Transmission electron microscopy (TEM, also sometimes conventional transmission electron microscopy or CTEM) is a microscopy technique in which a beam of electrons is transmitted through a specimen to form an image. The specimen is most often an ultrathin section less than 100 nm thick or a suspension on a grid. An image is formed from the interaction of the electrons with the sample as the beam is transmitted through the specimen. The image is then magnified and focused onto an imaging device, such as a fluorescent screen, a layer of photographic film, or a sensor such as a charge-coupled device. Transmission electron microscopes are capable of imaging at a significantly higher resolution than light microscopes, owing to the smaller de Broglie wavelength of electrons. This enables the instrument to capture fine detail, as small as a single column of atoms, which is thousands of times smaller than a resolvable object seen in a light microscope.

A scanning electron microscope (SEM) is a type of electron microscope that produces images of a sample by scanning the surface with a focused beam of electrons. The electrons interact with atoms in the sample, producing various signals that contain information about the surface topography and composition of the sample. The electron beam is scanned in a raster scan pattern, and the position of the beam is combined with the detected signal to produce an image. SEM can achieve resolution better than 1 nanometer.

Still further, Environmental SEM (ESEM) allowed samples to be observed in low-pressure gaseous environments (e.g., 1-50 Torr or 0.1-6.7 kPa) and high relative humidity (up to 100%). This was made possible by the development of a secondary-electron detector capable of operating in the presence of water vapor and by the use of pressure-limiting apertures with differential pumping in the path of the electron beam to separate the vacuum region (around the gun and lenses) from the sample chamber. ESEM is especially useful for non-metallic and biological materials because coating with carbon or gold is unnecessary.

X-ray crystallography is a technique used for determining the atomic and molecular structure of a crystal, in which the crystalline structure causes a beam of incident X-rays to diffract into many specific directions. By measuring the angles and intensities of these diffracted beams, a crystallographer can produce a three-dimensional picture of the density of electrons within the crystal. From this electron density, the mean positions of the atoms in the crystal can be determined, as well as their chemical bonds, their crystallographic disorder, and various other information.

Circular dichroism (CD) is dichroism involving circularly polarized light, i.e., the differential absorption of left- and right-handed light. Left-hand circular (LHC) and right-hand circular (RHC) polarized light represent two possible spin angular momentum states for a photon, and so circular dichroism is also referred to as dichroism for spin angular momentum. It is exhibited in the absorption bands of optically active chiral molecules. CD spectroscopy has a wide range of applications in many different fields. Most notably. UV CD is used to investigate the secondary structure of proteins.

Density functional theory (DFT) is a computational quantum mechanical modelling method used in physics, chemistry and materials science to investigate the electronic structure (principally the ground state) of many-body systems, in particular atoms, molecules, and the condensed phases. Using this theory, the properties of a many-electron system can be determined by using functionals, i.e., functions of another function, which in this case is the spatially dependent electron density. DFT is among the most popular and versatile methods available in condensed-matter physics, computational physics, and computational chemistry.

Dynamic light scattering (DLS) is a technique used to determine the size distribution profile of small particles in suspension or polymers in solution. In the scope of DLS, temporal fluctuations are usually analyzed by means of the intensity or photon autocorrelation function (also known as photon correlation spectroscopy or quasi-elastic light scattering). In the time domain analysis, the autocorrelation function (ACF) usually decays starting from zero delay time, and faster dynamics due to smaller particles lead to faster decorrelation of scattered intensity trace.

Ion-mobility spectrometry—mass spectrometry (IMS-MS), also known as ion-mobility separation-mass spectrometry, is an analytical chemistry method that separates gas phase ions on a millisecond timescale using ion-mobility spectrometry and uses mass spectrometry on a microsecond timescale to identify components in a sample. It should be noted that this method may be used for evaluating and measuring the levels of the metabolite and thereby for determining metabolite accumulation.

Metabolic Profiling. Metabolic profiling is a study of chemical processes that are associated to and involve metabolites. It is a study of chemical fingerprints that are very unique and that any specific physiological processes in a cell always leave behind.

Metabolic profiling can also be defined as the use of analytical methods in measurement and interpretation of various endogenous low molecular weight and intermediates from their samples. This study makes use of metabolome, and it provides a critical view of the physiological characteristic of a cell, tissue or the whole organism as compared to proteomic analysis and mRNA analysis. Metabonomics and metabolomics are other terms used in description of this study.

It should be understood that in some embodiments, the effect of the examined candidate on metabolite accumulation and/or on accumulation of pathologic protein associated with proteinopathies and/or protein misfolding disorder may be determined by additional parameters served herein as phenotype, specifically, when the candidate is evaluated using mammalian cells and specifically where a multicellular organism or a mammalian animal are used by the systems and methods of the invention as evaluation means. In yet some specific embodiments, such measured parameters may include morphology, motility, fertility, lethality, development, maturation, puberty, or any other behavioral or physiological parameters or phenotypes specifically associated with the particular protein misfolding disease. It should be appreciated that each and every one of the specified procedures and means is disclosed herein and forms a specific embodiment for each and every aspect of the present disclosure. It should be understood that any method and means described herein in connection with the systems of the invention may be also applicable for any of the methods of the invention and for any aspect disclosed herein.

As indicated above, the system of the invention may be suitable for screening of candidates for treating disorders associated with accumulation of at least one metabolite.

Metabolite, as used herein, is an organic compound which is an intermediate end product of metabolism or a metabolic process or pathway. A primary metabolite is directly involved in normal growth, development, and reproduction such as amino acid, nucleotide, carboxylic acid, alcohols, antioxidants, or vitamins. A secondary metabolite is not directly involved in those processes, but usually has an important ecological function such as pigments or antibiotics. In some specific embodiments, such metabolite may be any one of a nucleobase, an amino acid residue, carbohydrate, fatty acid and ketone, sterols, porphyrin and haem, lipid and lipoprotein, neurotransmitters, vitamins and (non-protein) cofactors, trace elements, metals, metabolites associated with energy metabolism, metabolites associated with peroxisome functions, or any intermediate product, derivative or metabolite thereof.

In more specific embodiments such metabolite may at least one amino acid residue, any derivative, any intermediate product thereof, or any combination or mixture thereof.

Amino acid is an organic compound containing amine (—NH2) and carboxyl (—COOH) functional groups, along with a side chain (R group) specific to each amino acid. As used herein, amino acid refers to a naturally occurring or synthetic amino acid, an amino acid analog, or an amino acid mimetic that functions in a manner similar to a naturally occurring amino acid.

Naturally occurring amino acids are those encoded by the genetic code, as well as those amino acids that are later modified, e.g., hydroxyproline, γ-carboxyglutamate, and O-phosphoserine.

In some embodiments, an amino acid or amino acid residue may refer to Arginine (denoted by Arg or R), Lysine (denoted by Lys or K), Aspartic acid (denoted by Asp or D), Glutamic acid (denoted by Glu or E), Glutamine (denoted by Gln or Q), Asparagine (denoted by Asn or N), Histidine (denoted by His or H), Serine (denoted by Ser or S), Threonine (denoted by Thr or T), Tyrosine (denoted by Tyr or Y), Cysteine (denoted by Cys or C), Tryptophan (denoted by Trp or W), Alanine (denoted by Ala or A), Isoleucine (denoted by Ile or I, Leucine (denoted by Leu or L), Methionine (denoted by Met or M), Phenylalanine (denoted by Phe or F). Valine (denoted by Val or V), Proline (denoted by Pro or P), Glycine (denoted by Gly or G).

The amino acids are characterized on the basis of their polarity, charge, solubility, hydrophobicity, hydrophilicity, and/or the amphiphathic nature. Nonpolar “hydrophobic” amino acids are such as valine, isoleucine, leucine, methionine, phenylalanine, tryptophan, cysteine, alanine, tyrosine, histidine, threonine, serine, proline, glycine, arginine and lysine: “polar” amino acids are such as arginine, lysine, aspartic acid, glutamic acid, asparagine, glutamine: “positively charged” amino acids are such as arginine, lysine and histidine: “acidic” amino acids are such as aspartic acid, asparagine, glutamic acid and glutamine; “aromatic” amino acids include tryptophan, tyrosine, naphthylalanine, and phenylalanine. About 500 naturally occurring amino acids are known (though only 20 appear in the genetic code) and can be classified in many ways. They can be classified according to the core structural functional groups' locations as alpha- (α-), beta- (β-), gamma- (γ-) or delta- (δ-) amino acids: other categories relate to polarity, pH level, and side chain group type (aliphatic, acyclic, aromatic, containing hydroxyl or sulfur, etc.).

Amino acid analogs are compounds that have the same fundamental chemical structure as naturally occurring amino acids, i.e., alpha carbon that is bound to a hydrogen, a carboxyl group, an amino group, and an R group, e.g., homoserine, norleucine, methionine sulfoxide, methionine methyl sulfonium. Such analogs have modified R groups or modified peptide backbones but retain the same basic chemical structure as a naturally occurring amino acid. Amino acid mimetics are chemical compounds that have a structure that is different from the general chemical structure of an amino acid, but that functions in a manner similar to a naturally occurring amino acid. Amino acids may be referred to herein by either their commonly known three letter symbols or by the one-letter symbols recommended by the IUPAC-IUB Biochemical Nomenclature Commission.

Amino acid analogs are such as homo-amino acids, N-alkyl amino acids, dehydroamino acids, aromatic amino acids and α,α-disubstituted amino acids, e.g., cystine, 5-hydroxylysine, 4-hydroxyproline, a-aminoadipic acid, a-amino-n-butyric acid, 3,4-dihydroxyphenylalanine, homoserine, α-methylserine, omithine, pipecolic acid, ortho, meta or para-aminobenzoic acid, citrulline, canavanine, norleucine, d-glutamic acid, aminobutvric acid, L-fluorenylalanine, L-3-benzothienylalanine and thyroxine.

In yet some further alternative embodiments, the metabolite may at least one nucleobase, any derivative, any intermediate product thereof, or any combination or mixture thereof.

Nucleobases, also known as nitrogenous bases or often simply bases, are nitrogen-containing biological compounds that form nucleosides, which in turn are components of nucleotides, with all of these monomers constituting the basic building blocks of nucleic acids. The ability of nucleobases to form base pairs and to stack one upon another leads directly to long-chain helical structures such as ribonucleic acid (RNA) and deoxyribonucleic acid (DNA).

Five nucleobases, adenine (A), cytosine (C), guanine (G), thymine (T), and uracil (U), are called primary or canonical. They function as the fundamental units of the genetic code, with the bases A, G, C, and T being found in DNA while A, G, C, and U are found in RNA. Thymine and uracil are identical excepting that T includes a methyl group that U lacks.

Adenine and guanine have a fused-ring skeletal structure derived of purine, hence they are called purine bases. Similarly, the simple-ring structure of cytosine, uracil, and thymine is derived of pyrimidine, so those three bases are called the pyrimidine bases. Each of the base pairs in a typical double-helix DNA comprises a purine and a pyrimidine: either an A paired with a T or a C paired with a G. These purine-pyrimidine pairs, which are called base complements, connect the two strands of the helix and are often compared to the rungs of a ladder. The pairing of purines and pyrimidines may result, in part, from dimensional constraints, as this combination enables a geometry of constant width for the DNA spiral helix. The A-T and C-G pairings function to form double or triple hydrogen bonds between the amine and carbonyl groups on the complementary bases.

It should be understood that the invention further encompasses any modified nucleobases, for example, modified adenosine or guanosine such as Hypoxanthine, anthine, Inosine, Xanthosine, 7-Methylguanosine (m⁷G), 7-Methylguanosine (m⁷G), or modified cytosine, thymine or uridine such as Dihydrouracil, 5-Methylcytosine, 5-Hydroxymethylcytosine, Dihydrouridine, 5-Methylcytidine.

Still further, Nucleosides are glycosylamines that can be thought of as nucleotides without a phosphate group. A nucleoside consists simply of a nucleobase (also termed a nitrogenous base) and a five-carbon sugar (either ribose or deoxyribose), whereas a nucleotide is composed of a nucleobase, a five-carbon sugar, and one or more phosphate groups. In a nucleoside, the base is bound to either ribose or deoxyribose via a beta-glycosidic linkage. Examples of nucleosides include cytidine, uridine, adenosine, guanosine, thymidine and inosine. Nucleotides, are organic molecules that serve as the monomer units for forming the nucleic acid polymers deoxyribonucleic acid (DNA) and ribonucleic acid (RNA), both of which are essential biomolecules within all life-forms on Earth. Nucleotides are the building blocks of nucleic acids; they are composed of three subunit molecules: a nitrogenous base, a five-carbon sugar (ribose or deoxyribose), and at least one phosphate group. A nucleoside is a nitrogenous base and a 5-carbon sugar. Thus a nucleoside plus a phosphate group yields a nucleotide.

In more specific embodiments such nucleobase may be at least one of purine nucleobases, any derivative or any intermediate product thereof. Purine is a heterocyclic aromatic organic compound that consists of a pyrimidine ring fused to an imidazole ring. There are many naturally occurring purines. They include the nucleobases adenine and guanine. Other notable purines are hypoxanthine, xanthine, theobromine, caffeine, uric acid and isoguanine. Aside from the crucial roles of purines (adenine and guanine) in DNA and RNA, purines are also significant components in a number of other important biomolecules, such as ATP, GTP, cyclic AMP, NADH, and coenzyme A.

In yet some further specific embodiments, such purine nucleobase may be at least one of adenine, and/or any derivative and intermediate thereof.

In some embodiments, the system of the present disclosure may be specifically appliable for any metabolite, for example, any one of an amino acid residue, a nucleobase, nucleoside, nucleotide, carbohydrate, fatty acid and ketone, sterols, porphyrin and haem, lipid, sphingolipid, phospholipid and lipoprotein, neurotransmitters, vitamins and (non-protein) cofactors, pterin, trace elements, metals, metabolites associated with energy metabolism, metabolites associated with peroxisome functions, or any intermediate product, derivative or metabolite thereof.

In some particular embodiment, the metabolite is at least one amino acid residue, any derivative, or any intermediate product or metabolite thereof.

In some specific embodiments, such amino acid residue or any intermediate product or metabolite thereof is at least one of: Homocysteine (Hcy), Phenylalanine. Tyrosine. Glycine, Arginine. Cysteine, Isoleucine. Leucine, Lysine, Methionine, Proline. Tryptophane, Valine, N-acetylaspartate (NAA), Homogentisic acid, and any derivatives thereof.

In yet some further specific embodiments such amino acid residue or any intermediate product or metabolite thereof may be at least one of: Homocysteine, Phenylalanine, Tyrosine, Glycine, Arginine, Cysteine, Isoleucine, Leucine, Lysine, Methionine, Proline, Tryptophane, Valine, N-acetylaspartate (NAA), Homogentisic acid, any branched-chain amino acid and any derivatives thereof. A branched-chain amino acid (BCAA) is an amino acid having an aliphatic side-chain with a branch (a central carbon atom bound to three or more carbon atoms). Among the proteinogenic amino acids, there are three BCAAs: leucine, isoleucine, and valine. Non-proteinogenic BCAAs include 2-aminoisobutyric acid.

It should be noted that Homogentisic acid, also known as melanic acid, is an intermediate in the breakdown or catabolism of tyrosine and phenylalanine.

Still further, N-Acetylaspartic acid, or N-acetylaspartate (NAA), is a derivative of aspartic acid.

In certain embodiments, the amino acid residue or any intermediate product or metabolite thereof is homocysteine. Homocysteine, as used herein, is a non-proteinogenic α-amino acid, also denoted as 2-Amino-4-sulfanylbutanoic acid. It is a homologue of the amino acid cysteine, differing by an additional methylene bridge (—CH₂—). It is biosynthesized from methionine by the removal of its terminal C^{ε} methyl group.

In such case, the yeast cell and/or yeast cell line and/or yeast cell population of the system provided herein, carry a genetic and/or epigenetic modification in the Cystathionine beta-synthase 4 (CYS4) yeast gene.

The CBS gene encodes an enzyme called cystathionine beta-synthase. This enzyme acts in a chemical pathway and is responsible for converting the amino acid homocysteine to a molecule called cystathionine. As a result of this pathway, other amino acids, including methionine, are produced. Mutations in the CBS gene disrupt the function of cystathionine beta-synthase, preventing homocysteine from being used properly. As a result, this amino acid and toxic byproducts substances build up in the blood. Some of the excess homocysteine is excreted in urine. Rarely, homocystinuria can be caused by mutations in several other genes. The enzymes encoded by the MTHFR, MTR, MTRR, and MMADHC genes play roles in converting homocysteine to methionine. Mutations in any of these genes lead to a accumulation of homocysteine in the blood.

As indicated herein, the system and methods of the invention may be particularly adapted for protein misfolding disorders involved with metabolites associated with amino acid metabolism. In accordance with such embodiments, the screening systems and methods of the invention disclosed herein after, may comprise yeast cell/s and/or yeast cell line/s, and/or yeast cell population/s, and/or any progeny thereof having manipulated and/or modified CYS4 gene. This genetic and/or epigenetic manipulation and/or modification leads to disruption of the CYS4 gene that results in accumulation of homocysteine. In some further embodiments, the system of the invention may comprise at least one genetically manipulated or modified yeast cell line that carry a modification in CYS4 yeast gene.

More specifically, the Cystathionine beta-synthase 4 (CYS4) yeast gene as used herein is the Cystathionine beta-synthase 4 (CYS4) in Saccharomyces cerevisiae (strain ATCC 204508/S288c or Baker's yeast) having the accession number NM_001181284.3 encodes for the enzyme Cystathionine beta-synthase or Beta-thionase or Serine sulfhydrase or Sulfur transfer protein 4 (having the accession number NP_011671.3).

In some embodiments, the gene CYS4 comprises the nucleic acid sequence as denoted by SEQ ID NO: 3. In some embodiments, the gene CYS4 encodes for a protein comprising the amino acid sequence as denoted by SEQ ID NO: 4.

Still further in some embodiments, the systems and methods of the present disclosure comprise yeast cell/s and/or yeast cell line/s, and/or yeast cell population/s, and/or any progeny thereof with a disrupted CYS4 gene. Still further, in some embodiments, the CYS4 gene is knocked out in the cells disclosed in the systems and methods. In some specific embodiments, the CYS4 knock out yeast cell/s and/or yeast cell line/s, and/or yeast cell population/s, and/or any progeny thereof, has been prepared by removal of the CYS4 open reading frame from the start codon to the stop codon. In some particular and non limiting embodiments, the yeast cells used herein are the Saccharomyces cerevisiae strain BY4741 [ATCC 4040002]. This fungal strain is also available as ATCC® Catalog No.: 201388™. The preparation of the CYS4 knock out strain is detailed in the experimental procedure section. In more specific embodiments the CYS4 Location is: Chromosome VII 798543.800066. In more specific embodiments the yeast cell/s of the invention, any cell line thereof, cell population or any progeny thereof, has deletion of the CYS4 gene open reading frame in Chromosome VII from position 798543 to position 800066. In yet some further embodiments, the yeast cell of the invention and/or any cell line, cell population and progeny thereof comprise selection markers that replace the open reading frames from the start codon to the stop codon. It should be understood that the specific manipulated yeast cell of the invention or any population, cell line or progeny thereof are provided by the invention and used herein by the systems and methods described herein only as non-limiting embodiments. It must be understood that any genetic and/or epigenetic manipulation and/or modification, for example, mutation, deletion or insertion to any part or portion of the gene may be performed. For example, only part of the open reading frame (10%, 20%, 30%, 405<50%, 60%, 70%, 80%, 90% and more 95% and more) can be deleted, mutated or otherwise modified (including epigenetic modifications such as methylation and the like), provided that such manipulation/s and/or modification/s led to accumulation of homocysteine. In yet some further embodiments, the manipulation may be either transient, and/or stable and/or inducible.

In more specific embodiments, the genetically and/or epigenetically modified yeast cell and/or yeast cell line, and/or yeast cell population, display reduced or no expression of CYS4 gene, thereby displaying accumulation of Homocysteine and any derivative thereof.

In yet some alternative or additional embodiments, the system disclosed herein is applicable for a metabolite such as an amino acid residue or any intermediate product or metabolite thereof, specifically. Homocysteine. According to such embodiments, the yeast cell and/or yeast cell line, and/or yeast cell population provided by the disclosed system grow under conditions of Homocysteine supplementation, optionally, supplemented to the growth medium thereof.

The present disclosure provides systems and methods that are specifically directed for screening and identification of therapeutic compounds applicable for the treatment of proteinopathies and/or protein misfolding disorders. The term Proteinopathy, as used herein, refers to protein conformational disorder, or protein misfolding disease and relates to a class of diseases in which certain proteins become structurally abnormal, and thereby disrupt the function of cells, tissues and organs of the body. Often the proteins fail to fold into their normal configuration; in this misfolded state, the proteins can become toxic in some way (a toxic gain-of-function) or they can lose their normal function. The proteinopathies include such diseases as Creutzfeldt-Jakob disease and other prion diseases, Alzheimer's disease, Parkinson's disease, amyloidosis, multiple system atrophy, and a wide range of other disorders. More specifically, “Protein misfolding and aggregation” as used herein, relates to an impaired physical process by which a protein chain acquires its native three-dimensional structure, a conformation that is usually biologically functional, in an expeditious and reproducible manner. It is the physical process by which a polypeptide folds into its characteristic and functional three-dimensional structure from random coil. Each protein exists as an unfolded polypeptide or random coil when translated from a sequence of mRNA to a linear chain of amino acids. Amino acids interact with each other to produce a well-defined three-dimensional structure, the folded protein, known as the native state. The correct three-dimensional structure is essential to function, although some parts of functional proteins may remain unfolded. Failure to fold into native structure generally produces inactive proteins, but in some instances misfolded proteins have modified or toxic functionality. Several neurodegenerative and other diseases are believed to result from the accumulation of amyloid fibrils formed by the association of misfolded proteins.

More specifically, under some conditions, proteins may not fold into their biochemically functional forms resulting in protein denaturation. A fully denatured protein lacks both tertiary and secondary structure and exists as a so-called random coil. Under certain conditions some proteins can refold; however, in many cases, denaturation is irreversible. Cells sometimes protect their proteins against the denaturing influence of heat with enzymes known as chaperones or heat shock proteins, which assist other proteins both in folding and in remaining folded. Some proteins never fold in cells at all except with the assistance of chaperone molecules, which either isolate individual proteins so that their folding is not interrupted by interactions with other proteins or help to unfold misfolded proteins, giving them a second chance to refold properly. This function is crucial to prevent the risk of precipitation into insoluble amorphous aggregates.

Aggregated proteins are associated with prion-related illnesses such as Creutzfeldt-Jakob disease, bovine spongiform encephalopathy (mad cow disease), amyloid-related illnesses such as Alzheimer's disease and familial amyloid cardiomyopathy or polyneuropathy, as well as intracytoplasmic aggregation diseases such as Huntington's and Parkinson's disease. These age onset degenerative diseases are associated with the aggregation of misfolded proteins into insoluble, extracellular aggregates and/or intracellular inclusions including cross-beta sheet amyloid fibrils. It is not completely clear whether the aggregates are the cause or merely a reflection of the loss of protein homeostasis, the balance between synthesis, folding, aggregation and protein turnover. Misfolding and excessive degradation instead of folding and function leads to a number of proteopathy diseases such as antitrypsin-associated emphysema, cystic fibrosis and the lysosomal storage diseases, where loss of function is the origin of the disorder.

As some of the conditions associated with protein misfolding and protein aggregations involve neurodegeneration, in certain specific embodiments, the methods and systems of the present disclosure may be applicable for neurodegenerative diseases.

Still further, in some embodiments, the system provided herein is specifically applicable for screening for candidate therapeutic compounds useful for treating at least one proteinopathy and/or protein misfolding and/or protein aggregation disorder. In some embodiments, such proteinopathy or protein misfolding disorder may be a neurodegenerative disorder or any early signs or symptoms associated therewith.

The term “neurodegenerative diseases” is the general term for the progressive loss of structure or function of neurons, leading to their death. The greatest risk factor for neurodegenerative diseases is aging. Mitochondrial DNA mutations as well as oxidative stress both contribute to aging. Many of these diseases are late-onset, meaning there is some factor that change as a person ages, for each disease. One constant factor is that in each disease, neurons gradually lose function as the disease progresses with age.

In some specific embodiments, the proteinopathy and/or neurodegenerative disorder is a disorder characterized by at least one of beta-amyloid protein aggregation, an alpha synuclein, and/or beta-synuclein aggregates, islet amyloid polypeptide aggregates, TAR DNA-binding protein 43 aggregates, huntingtin aggregates, serum amyloid protein aggregates, and tau protein aggregation.

Thus, it should be appreciated that in certain embodiments, the invention may be further applicable for disorders characterized by beta-amyloid protein aggregation.

A group of disorders associated with beta-amyloid protein aggregation include Alzheimer's disease (AD), where deposits of a protein precursor called beta-amyloid build up (termed plaques) in the spaces between nerve cells and twisted fibers of tau protein build up (termed tangles) inside the cells.

In yet some further embodiments, the screening systems and methods disclosed herein are applicable for identifying therapeutic compounds for disorders associated with accumulation and/or aggregation of beta-amyloid protein. More specifically. “Beta-amyloid protein aggregations” as used herein relates to cerebral plaques laden with β-amyloid peptide (A) and dystrophic neurites in neocortical terminal fields as well as prominent neurofibrillary tangles in medial temporal-lobe structures, which are important pathological features of Alzheimer's disease. Subsequently, loss of neurons and white matter, congophilic (amyloid) angiopathy are also present.

As peptides are natural products of proteolysis of a precursor and are in a length ranging between 36 to 43 amino acids. Monomers of Aβ40 are much more prevalent than the aggregation-prone and damaging Aβ42 species. Still further, in some embodiments, the human Amyloid-beta precursor protein, as denoted by P05067 (also denoted by SEQ ID NO. 5). More specifically. β-amyloid peptides originate from proteolysis of the amyloid precursor protein by the sequential enzymatic actions of beta-site amyloid precursor protein-cleaving enzyme 1 (BACE-1), a β-secretase, and γ-secretase, a protein complex with presenilin 1 at its catalytic core. An imbalance between production and clearance, and aggregation of peptides, causes Aβ to accumulate, and this excess is considered as the initiating factor in Alzheimer's disease. β-amyloid can also grow into fibrils, which arrange themselves into β-pleated sheets to form the insoluble fibers of advanced amyloid plaques. Soluble oligomers and intermediate amyloid are the most neurotoxic forms of Aβ. In brain-slice preparations, dimers and trimers of Aβ are toxic to synapses. Experimental evidence indicates that Aβ accumulation precedes and drives tau protein aggregation.

In yet some further embodiments, the screening systems and methods disclosed herein are applicable for identifying therapeutic compounds for disorders associated with accumulation and/or aggregation of Tau protein. More specifically, “Tau protein” as used herein, refers to a protein participating in neurofibrillary tangles, which are filamentous inclusions in pyramidal neurons, characteristic for Alzheimer's disease and other neurodegenerative disorders termed tauopathies. Elucidation of the mechanisms of their formation may provide targets for future therapies. Accumulation of hyperphosphorylated Tau protein, that in some embodiments is the human Microtubule-associated protein tau (standard), as disclosed by spP10636. In some embodiments, the Tau protein comprises the amino acid sequence as denoted by SEQ ID NO: 6, as paired helical filaments in pyramidal neurons is a major hallmark of Alzheimer disease (AD). In yet some further alternative or additional embodiments, the Tau protein may be the human Tau protein that comprises the amino acid sequence as denoted by SEQ ID NO: 7. Besides hyperphosphorylation, other modifications of the Tau protein, such as cross-linking, are likely to contribute to the characteristic features of paired helical filaments, including their insolubility and resistance against proteolytic degradation. These neurofibrillary tangles, consist of hyperphosphorylated and aggregated forms of the microtubule-associated protein tau.

Under nonpathological conditions, tau is a developmentally regulated phosphoprotein that promotes assembly and stability of microtubules and is thus involved in axonal transport. In AD and other tauopathies, tau proteins aggregate and form fibrillar insoluble intracellular inclusions, so-called neurofibrillary tangles. It has been suggested that ionic interactions and covalent cross-linking contribute to pathological Tau aggregation and tangle formation. Reactive carbonyl compounds, which are increased under conditions of oxidative stress and in aging have been proposed as potential compounds responsible for tau aggregation. As indicated above, in some embodiments, the systems and methods of the present disclosure may be applicable for AD. Alzheimer's disease (AD) is a progressive neurodegenerative disease. It is the most prevalent form of dementia in the world affecting almost 47 million people worldwide (Alzheimer's Association). The number of Alzheimer's patients is projected to reach 82 million globally by 2030, and the number is expected to rise to 152 million by 2050 of which Asia-Pacific alone shall contribute 71 million cases. The two main pathological hallmarks of the disease include Aβ plaques and neurofibrillary tangles. Other clinical features include depression, hallucinations, speech impairment, motor disabilities and aggressive behavior. Though extensive research has been done, yet, early diagnosis of AD is still not possible. The latter stages of AD include significant neuronal loss in specific regions of the brain, ultimately leading to shrinkage of the total volume of the brain. AD can be categorized into two main types, familial AD (FAD) and sporadic AD (SAD). FAD shows an autosomal dominant inheritance and is usually caused by mutations occurring mainly in three genes, Amyloid precursor protein (APP), Presenilin 1 (PSEN1) and Presenilin 2 (PSEN 2). FAD accounts for only about 1-5% cases of AD, whereas the rest is attributed by SAD. SAD is attributed mainly by a combination of environmental risk factors and genetic susceptibility. Reports suggest a functional role of Apolipoprotein E (ApoE) phenotype in the late-onset AD, ε4 allele being the major risk factor for AD, whereas ε2 allele is protective. Aβ is generated from APP which is a type I transmembrane glycoprotein. β- and γ-secretases participate in the cascade cleavage of APPβ to form Aβ peptides, whereas α- and γ-secretases are involved in P3 peptide production from APPα. Various isoforms of Aβ with different lengths are produced, especially the toxic peptides Aβ42 and Aβ40. Oligomers, protofibrils, and fibrils generated by the accumulation of Aβ finally lead to Aβ plaque formation.

Accordingly, in some embodiments of the systems disclosed herein, the pathological protein is at least one of beta-amyloid protein, alpha synuclein and/or beta-synuclein, islet amyloid polypeptide, TAR DNA-binding protein 43 (TDP-43), Superoxide dismutase (SOD1), Leucine Rich Repeat Kinase 2 (LRRK2), Amyloid-beta precursor protein (APP), ataxin-3, huntingtin protein, serum amyloid proteins and tau protein.

Still further, in some embodiments, the system of the invention may be applicable for screening of therapeutic compounds applicable in any beta-amyloid protein aggregation disorder or tauopathy, for example, any one of Alzheimer's disease (AD) and age-associated cognitive decline (ACD). In yet some further embodiment, the system disclosed herein may be useful in screening for compounds applicable in the treatment of any alpha-synuclein pathology, for example, at least one of Parkinson disease (PD), Dementia with Lewy Bodies (DLB) and multiple system atrophy (MSA). Still further, in some embodiments, the systems disclosed herein, as well as the methods disclosed herein after, are applicable for any TAR DNA-binding protein 43 pathology, specifically, at least one of amyotrophic lateral sclerosis (ALS). Still further, in some embodiments, the systems disclosed herein, as well as the methods disclosed herein after, are applicable for any huntingtin protein pathology, specifically. Huntington disease. In yet some further embodiments, the systems disclosed herein, as well as the methods disclosed herein after, are applicable for any islet amyloid polypeptide pathology, for example, Type 2 diabetes. In some further embodiments, the systems disclosed herein, as well as the methods disclosed herein after, are applicable for any serum amyloid protein pathology, for example, systemic amyloidosis. Still further, in some embodiments, the present disclosure is further applicable for any cognitive disorders. “Age-associated mild cognitive impairment (MCI)”, as use herein is a condition that causes cognitive changes. MCI that primarily affects memory may be classified as “amnestic MCI” where the subjects experience impairment in memorizing information that relate to recent events, appointments or conversations or recent events. MCI that affects thinking skills other than memory is known as “nonamnestic MCI”. Thinking skills that may be affected by nonamnestic MCI include the ability to make sound decisions, judge the time or sequence of steps needed to complete a complex task, or visual perception. Normal aging is associated with a decline in various memory abilities in many cognitive tasks; the phenomenon is known as age-related memory impairment (AMI), age-associated memory impairment (AAMI) or age-associated cognitive decline (ACD).

Still further, in some further embodiments, the screening systems and methods disclosed herein are applicable for identifying therapeutic compounds for disorders associated with accumulation and/or aggregation of alpha synuclein. In some embodiments, alpha-synuclein comprises the amino acid sequence as denoted by SEQ ID NO: 8.

“Alpha-synuclein pathology disorders” or “Synucleinopathies” is used to name a group of neurodegenerative disorders characterized by fibrillary aggregates of alpha-synuclein protein in the cytoplasm of selective populations of neurons and glia. More specifically, as used herein are disorders characterized by the presence of a specific intracellular protein aggregates (inclusion bodies) known as Lewy bodies that contain mainly alpha-synuclein protein. Alpha-synuclein protein is found naturally as an unfolded cytoplasmic protein in neuronal synaptic areas.

Overexpression of alpha-synuclein interrupts normal cell functions and leads to decreases in neurite outgrowth and cell adhesion. Alpha-synuclein aggregates comprising monomeric, oligomeric intermediate, or fibrillar forms are thought to be involved in a critical step in the pathogenesis of Parkinson's disease (PD) and in other alpha-synucleinopathies, such as multiple system atrophy (MSA) and dementia with Lewy bodies (DLB). These chronic neurodegenerative diseases of the CNS are characterized by the development of Lewy bodies containing alpha-synuclein protein. Oligomeric and monomeric alpha-synuclein have both been detected in cerebrospinal fluid and plasma samples from PD patients, suggesting that small aggregates of alpha-synuclein access the extracellular space. Previous animal and clinical data suggest that misfolded alpha-synuclein can be released from cells by exocytosis and transmitted from one brain area to another via cell-to cell propagation. Although the exact mechanism of alpha-synuclein transmission remains unknown, evidence suggests that clathrin-mediated endocytosis (CME) may have an important role in internalization of extracellular alpha-synuclein. As the cargo protein for endocytosis is usually recognized by a specific receptor on the cell surface, it is possible that alpha-synuclein may interact with cell-surface receptors that have not been well specified until now. N-methyl-D-aspartate (NMDA) receptor subunits contain motifs that bind the endocytic adaptor protein involved in CME. Additionally, a recent study provided the evidence that alpha-synuclein could promote endocytic internalization of surface NMDA receptors through a mechanism requiring clathrin, suggesting an interaction between alpha-synuclein and NMDA receptors. Accordingly, alpha-synuclein propagation from one area of the brain to others via cell-to-cell transmission is closely related with disease progression or clinical severity. Still further. Lewy body pathology in Parkinson's disease also found in peripheral nervous system. In neurons innervating the gastrointestinal tract and appendix. Peripheral Lewy pathology is suggested to precede the CNS Lewy pathology and according to Braak hypothesis, precede disease onset.

More specifically, “Parkinson's disease (PD)” as used herein, is a neurodegenerative disease resulting from degeneration of midbrain dopamine neurons and accumulation of alpha-synuclein containing Lewy bodies in surviving neurons. The diagnosis of PD is based on the presence of cardinal motor features in the absence of other aetiological conditions. These motor features include the classical triad of bradykinesia, a resting pill-rolling tremor, and rigidity typically in association with hypomimia, hypophonia, micrographia and postural instability. Non-motor features of PD may even precede its diagnosis, constituting prodromal or premotor PD. These premotor features include problems with olfaction, constipation, mood and sleep, and following the clinical diagnosis of PD, they can become more prominent. Cognitive problems and dementia also commonly develop in PD, affecting almost 50% by 10 years from diagnosis. However, in some individuals with an alpha-synucleinopathy, significant cognitive problems precede the onset of parkinsonian motor symptoms, and these cases are clinically classified with a diagnosis of Dementia with Lewy Bodies. There is clearly a major degree of overlap between these two conditions both clinically and pathologically, but at present, the clinical distinction rests on the time interval between the onset of motor symptoms and dementia, with a minimum one year interval being required for a diagnosis of PD as opposed to Lewy body dementia (DLB).

Multiple system atrophy (MSA) is the rarest of the three major alpha synucleinopathies and differs significantly from PD and DLB in terms of its clinical presentation and its more aggressive course, reflecting differences in the underlying neuroanatomical pathways involved.

In yet some further embodiments, the invention may be applicable for DLB. More specifically, “Dementia with Lewy Bodies (DLB)”, as used herein, is a relatively common cause of dementia, estimated to account for up to 30% of dementia cases, and affecting up to 5% of those over the age of 75. Pathologically, it is defined by the presence of alpha synuclein containing Lewy bodies in the brain, but their distribution differs from that in PD, affecting the neocortex, limbic system and brainstem, in contrast to the nigrostriatal and brainstem-predominant pattern seen in early PD.

In yet some further embodiments, the invention may be applicable for MSA. “Multiple system atrophy (MSA)”, as used herein, is much rarer than PD with an estimated prevalence of 4.4 per 100 000 (PD is around 45 times more common).

In some specific embodiments, the present disclosure provides a yeast screening system of candidate therapeutic compounds for treating, preventing, ameliorating, reducing or delaying the onset of at least one disorder characterized by at least one of beta-amyloid protein aggregation. More specifically, such system comprises: (a) a yeast cell and/or yeast cell line, and/or yeast cell population, that display accumulation of Homocysteine. In some embodiments, wherein at least one of: (i) the yeast cell/s carry at least one manipulation and/or modification in at least one yeast metabolic pathway that leads to accumulation of the homocysteine (Hcy); (ii) the yeast cell/s grow under conditions that result in accumulation of the Hcy; and (iii) the yeast cell/s endogenously and/or exogenously express at least one pathologic protein associated with beta-amyloid protein aggregation. In some optional embodiments, the system provided herein may further comprise (b), at least one reagent or means for determining at least one of, the accumulation of the Hcy, and/or at least one phenotype associated with accumulation of the Hcy and any derivative thereof, and/or misfolding of the beta-amyloid protein.

It is to be appreciated that by providing systems and methods for identifying therapeutic compounds applicable for various proteinopathies, by identifying compounds that disturb the formation of metabolite fibril/s that enhance and initiate the formation of assemblies of pathogenic proteins involved with proteinopathies and protein misfolding disorders, the present disclosure provide systems and methods for identifying compounds useful in inhibiting and disturbing the formation of assemblies of such pathogenic proteins. In specific and non-limiting embodiments, the present disclosure provides systems and methods for treating AD, by identifying compounds that inhibit Hcy-fibril formation. As such, the invention provides methods and systems for inhibiting assemblies of pathogenic proteins, for example, beta amyloids and/or any of the disclosed proteins associated with proteinopathies. For example, the present disclosure provides methods and systems for the inhibition, reduction, elimination, attenuation, retardation, decline, prevention or decrease of assembly formation and/or fibril formation, and/or aggregation of at least one of the disclosed pathogenic proteins, specifically, at least one of beta-amyloid protein, alpha synuclein and/or beta-synuclein, islet amyloid polypeptide, TAR DNA-binding protein 43 (TDP-43), Superoxide dismutase (SOD1), Leucine Rich Repeat Kinase 2 (LRRK2), Amyloid-beta precursor protein (APP), ataxin-3, huntingtin protein, serum amyloid proteins and tau protein, in or by at least about 5%-99.9999%, specifically, at least about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 9%, 97%, 98%, 99%, 99.9%, 99.99%, 99.999%, 99.9999% or about 100%, of the assembly formation and/or fibril formation, and/or aggregation caused in a diseased and/or untreated subject, tissue or state. Still further, in some further embodiments, the screening systems and methods disclosed herein are applicable for identifying therapeutic compounds for disorders associated with accumulation and/or aggregation of beta synuclein. In some embodiments, beta-synuclein is denoted by Q16143.1. Still further, the beta synuclein disclosed herein may comprise the amino acid sequence as denoted by SEQ ID NO. 9. Still further, in some further embodiments, the screening systems and methods disclosed herein are applicable for identifying therapeutic compounds for disorders associated with accumulation and/or aggregation of Huntingtin. In some embodiments, Huntingtin is denoted by P42858. Still further, the Huntingtin disclosed herein may comprise the amino acid sequence as denoted by SEQ ID NO: 10. Still further, in some further embodiments, the screening systems and methods disclosed herein are applicable for identifying therapeutic compounds for disorders associated with accumulation and/or aggregation of Ataxin-3. In some embodiments, Ataxin-3 is denoted by P54252. Still further, the Ataxin-3 disclosed herein may comprise the amino acid sequence as denoted by SEQ ID NO: 11. Still further, in some further embodiments, the screening systems and methods disclosed herein are applicable for identifying therapeutic compounds for disorders associated with accumulation and/or aggregation of Leucine-rich repeat serine/threonine-protein kinase 2 (LRRK2). In some embodiments, LRRK2 is denoted by Q5S007. Still further, the LRRK2 disclosed herein may comprise the amino acid sequence as denoted by SEQ ID NO: 12. Still further, in some further embodiments, the screening systems and methods disclosed herein are applicable for identifying therapeutic compounds for disorders associated with accumulation and/or aggregation of TAR DNA-binding protein 43. In some embodiments, TAR DNA-binding protein 43 is denoted by Q13148. Still further, the TAR DNA-binding protein 43 disclosed herein may comprise the amino acid sequence as denoted by SEQ ID NO: 13. Still further, in some further embodiments, the screening systems and methods disclosed herein are applicable for identifying therapeutic compounds for disorders associated with accumulation and/or aggregation of Superoxide dismutase [Cu—Zn] (SOD1). In some embodiments, SOD1 is denoted by P00441. Still further, the TAR SOD1 disclosed herein may comprise the amino acid sequence as denoted by SEQ ID NO: 14. Still further, in some further embodiments, the screening systems and methods disclosed herein are applicable for identifying therapeutic compounds for disorders associated with accumulation and/or aggregation of Islet amyloid polypeptide. In some embodiments. Islet amyloid polypeptide is denoted by P10997. Still further, the Islet amyloid polypeptide disclosed herein may comprise the amino acid sequence as denoted by SEQ ID NO: 15.

As firstly showed in the present disclosure, the inventors revealed the connection between accumulation and aggregation of metabolites, e.g., Hcy, and initiation of accumulation and/or aggregation of pathogenic proteins such as amyloid beta, that is associated with proteinopathies. Thus, in some embodiments, the methods of the invention screen for candidate compound/s that modulate the level of the accumulation of the metabolite and/or the level of a phenotype, specifically toxic phenotype, associated with accumulation of the metabolite and more importantly, reduce the accumulation and/or aggregation of the pathologic protein associated with proteinopathies. Such compound may be particularly useful in treatment of proteinopathies and/or protein misfolding disorders. “Modulate” as used herein means to decrease (e.g., inhibit, reduce, suppress) or alternatively, increase (e.g., stimulate, activate, enhance) a level, response, property, activity, pathway, or process. A “modulator”, as used herein when referred to the candidate compound tested or evaluated by the systems and methods of the invention, is compound capable of modulating a level of the accumulation of the metabolite and/or the level of a phenotype associated with said accumulation. A modulator may be an inhibitor, antagonist, activator, or agonist. In some embodiments modulation may refer to an alteration, e.g., inhibition or increase, of the of the accumulation of the metabolite and/or the level of a phenotype associated with said accumulation by at least about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99% or 100%. Specifically, as compared with the level of the accumulation of the metabolite and/or the level of a phenotype associated with said accumulation in the absence of such modulator.

In some embodiments, modulated refers to either reduced or elevated. In yet some further specific embodiments, the candidate a therapeutic compound for the protein misfolding disorder if the level of said phenotype is reduced as compared with the level of the phenotype in the absence of said candidate compound.

In more specific embodiments, a decrease or inhibition of growth or viability indicates toxicity of the accumulated metabolite and/or pathologic protein associated with protein misfolding disorders, in the yeast cell or in the cell, unicellular organism, multicellular organism or mammal of the validation means described herein. Toxicity of the accumulated metabolite and/or pathologic protein associated with protein misfolding disorders, in yeast correlates with human and/or other mammalian proteinopathies and/or protein misfolding disorder state associated with abnormal accumulation, and/or aggregation, of the metabolite and/or pathologic protein associated with protein misfolding disorders. If such a yeast cell is exposed to a candidate compound, one can test the ability of the compound to modulate, e.g., inhibit, toxicity in the cell by measuring growth or viability of the cell and comparing the growth or viability with the growth or viability of a yeast cell cultured in the absence of the compound. For example, a screen may be performed that comprises culturing yeast cells that carry at least one manipulation in at least one pathway involved in the metabolism of the metabolite in the presence of a compound, measuring cell growth or viability in the presence of the compound, and comparing cell growth or viability measured in the presence of the compound to cell growth or viability in the absence of the candidate compound. If cell growth or viability is increased or decreased in the presence of the compound as compared to cell growth or viability in the absence of the compound, the compound is identified as a compound that modulates toxicity induced by the accumulated metabolite and/or pathologic protein associated with protein misfolding disorders.

A further aspect of the preset disclosure relates to a screening method of candidate therapeutic compounds for treating, preventing, ameliorating, reducing or delaying the onset of at least one protein misfolding disorder. More specifically, the method comprising the following steps: in a first step (a), contacting a manipulated and/or modified yeast cell/s and/or yeast cell line/s, and/or yeast cell population/s that display accumulation of at least one metabolite, with a candidate compound. In some embodiments, the cells contacted in (a) may be characterized by one of; in some embodiments (i), the yeast cell/s carny at least one manipulation and/or modification in at least one yeast metabolic pathway that leads to accumulation of the metabolite. In alternative or additional embodiments (ii), the yeast cell/s grow under conditions that result in accumulation of said metabolite. In some further additional or alternative embodiments (iii), the yeast cell/s endogenously and/or exogenously express at least one pathological protein associated with the proteinopathy and/or protein misfolding disorder. The next step (b), involves determining in the contacted cells of step (a), at least one of: (i) the accumulation of the metabolite; (ii) the level and/or manifestation of at least one phenotype associated with the accumulation of the metabolite and/or the pathologic protein; and/or (iii) the accumulation of the pathological protein. The next step (c), involves determining that the candidate is a therapeutic compound for the protein misfolding disorder if a change and/or modulation is detected and/or observed and/or determined in at least one of the following parameters: (i) the accumulation of the metabolite; and/or (ii) the accumulation of the pathological protein; and/or (iii) the observed phenotype, in the cells, as compared with the accumulation of the metabolite, and/or accumulation of the pathological protein and/or the observed phenotype in the absence of the candidate compound. Thus, by comparing the indicted parameters in cells treated with the candidate compound to untreated cells, any change and/or modulation in any of the indicated parameters may indicate that the candidate compound may be potentially used as the therapeutic compound, or in other words, may be a potential therapeutic compound.

In some embodiments a system and method of screening for a compound that decreases toxicity associated with accumulation of a specific metabolite and/or pathologic protein associated with protein misfolding disorders, may comprise: contacting a yeast cell that carry at least one manipulation in at least one pathway involved in the metabolism of said metabolite with a test compound; and evaluating the yeast cell for viability, wherein an increase in viability of the yeast cell as compared to viability of the yeast cell in the absence of the compound indicates that the compound decreases toxicity associated with accumulation of the specific metabolite and/or pathologic protein, in a specific protein misfolding disorder. In some embodiments, compound that inhibit toxicity associated with the accumulated metabolite are candidate therapeutic compounds for treating any proteinopathy and/or any protein misfolding disorder characterized by accumulation of the metabolite. In certain embodiments the metabolite accumulates and in further embodiments, forms detectable aggregates in the yeast cell. Compounds can be tested for their ability to modulate, e.g., inhibit, formation or persistence of metabolite aggregates, or alternatively, inhibit the accumulation of the metabolite, or even reduce, at least in part the level of the metabolite.

In yet some further specific embodiments, a screen may be performed that comprises culturing yeast cells that carry at least one manipulation in at least one pathway involved in the metabolism of the metabolite, for example. Hcy, in the presence of a compound, measuring metabolite accumulation and/or metabolite aggregation, and/or aggregation and/or accumulation of pathologic protein associated with protein misfolding disorders, e.g., amyloid beta, in the presence of the compound, and comparing metabolite accumulation and/or metabolite aggregation measured in the presence of the compound to metabolite accumulation and/or metabolite aggregation in the absence of the candidate compound. If metabolite accumulation and/or metabolite aggregation is increased or decreased in the presence of the compound as compared to metabolite accumulation and/or metabolite aggregation in the absence of the compound, the compound is identified as a compound that modulates metabolite accumulation (level) and/or metabolite aggregation of the accumulated metabolite. In some embodiments a method of screening for a compound that decreases accumulation of a specific metabolite or formation of metabolite aggregate may comprise: contacting a yeast cell that carry at least one manipulation in at least one pathway involved in the metabolism of the metabolite with a compound; and evaluating the yeast cell for metabolite level and/or metabolite aggregation, wherein a decrease in metabolite accumulation and/or metabolite aggregation of the yeast cell as compared to metabolite accumulation or level and/or metabolite aggregation and/or accumulation or aggregation of pathologic protein associated with protein misfolding disorders, in the yeast cell in the absence of the compound indicates that the compound decreases toxicity associated with accumulation of the specific metabolite in a specific protein misfolding disorder. In some embodiments, compound that inhibit toxicity and/or metabolite aggregation associated with the accumulated metabolite by at least about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99% or about 100%, are candidate therapeutic compounds that may be suitable for treating an protein misfolding disorder characterized by accumulation of the metabolite.

It should be understood that the yeast cells are indicated herein for simplicity, since this step is mandatory. However, when the methods of the invention further comprise validation step, similar comparison of the effect of the tested candidate on accumulation of the metabolite and/or accumulation or aggregation of pathologic protein associated with protein misfolding disorders, and/or the level of the phenotype associated with the accumulation, also in the cell, unicellular organism, multicellular organism or mammal of the validation means described herein, is to be compared to the effect in the absence of the candidate compound.

In some specific embodiments, the phenotype associated with accumulation of the metabolite determined or evaluated by the methods of the invention may be at least one of cell toxicity and formation of metabolite aggregate.

In yet some further embodiments of the methods of the invention, cell toxicity is determined by the method of the invention by measuring at least one of cell viability, cell proliferation, cell apoptosis and any toxic phenotype on the organism or cell.

As mentioned herein in connection with other aspects of the invention, in some specific embodiments, cell viability may be determined by 2,3-bis-(2-methoxy-4-nitro-5-sulphophenyl)-2H-tetrazolium-5-carboxanilide (XTT) viability assay (not useful for cysteine accumulation), Methylene Blue, PrestoBlue viability reagent, the fluorescent intercalator 7-aminoactinomycin D (7-AAD), LIVE/DEAD Viability Kits, cell growth by turbidity, for example at OD600, or by any means for cell counting.

Thus, in some embodiments, the methods of the invention may comprise at least one step required for performing any cell viability and proliferation assay, specifically, any of the assays disclosed above.

In yet some further embodiments, toxicity may be evaluated by measuring apoptosis of the cells. In some embodiments, apoptosis may be determined by at least one of DNA fragmentation (TUNNEL, terminal deoxynucleotidyl transferase dUTP nick end labeling), caspase and or parp1 phosphorylation, annexin V and propidium iodide (PI) assay. Thus, in some embodiments, the methods of the invention may comprise at least one step required for performing any apoptosis or any other cell death assay, specifically, any of the assays disclosed above.

It should be noted that in some embodiments, other toxic phenotype that may be determined on the organism/cell may include, activation of cell stress pathways (e.g., heat shock response), poor fertility, destruction of organs and tissue damages, DNA mutagenesis, ER stress, cell energy status and ATP content, oxidative stress, mitochondrial dysfunction, mitochondrial damage, activation of autophagy and activation of necrosis.

Thus, in some embodiments, the methods of the invention may further comprise at least one step required for performing any of the cell toxicity assay disclosed above.

In yet some further embodiments, metabolite accumulation and/or formation of metabolite aggregation may be determined by the method of the invention using at least one of microscopy, light diffraction, absorption, or scattering assay, spectrometric assay, immunological assay, Liquid Chromatography. NMR, flow cytometry, and stereoscopy. In yet some further embodiments, metabolite accumulation and/or metabolite aggregation may be measured using at least one of Dye-binding specificity (for example, using thioflavin T (ThT) and congo red, microscopy, X-ray fiber diffraction, X-ray powder diffraction, X-ray single crystal diffraction, mass spectrometry (including ion mobility), immunological assay (e.g. using a specific antibody), flow cytometry, circular dichroism (CD) spectrometry, vibrational CD, Raman Spectroscopy, Fourier-transformed infrared spectroscopy dynamic light scattering (DLS), Liquid Chromatography (HPLC and UPLC) and NMR. Microscopy, metabolic profiling, such as TEM (transmission electron microscope), confocal fluorescence microscopy, confocal Raman microscopy, indirect immunofluorescence.

In some embodiments, the methods disclosed herein may further comprise the step of further validating a candidate compound determined as a potential therapeutic compound displaying a change or modulation in at least one of: (i) the accumulation of the metabolite and/or accumulation of the pathological protein and/or change or modulation in the observed phenotype as determined in step (c), of the disclosed method. The additional validation and evaluation may involve in some embodiments the use of various cellular and/or in vitro and/or in vivo systems and/or platforms. The validation therefore may comprise according the following steps: First (I), contacting the candidate compound with at least one of: in some embodiments (i), at least one unicellular organism that display accumulation of the metabolite and/or accumulation of the pathological protein; in yet some further alternative or additional embodiments (ii), at least one multicellular eukaryotic organism that display accumulation of the metabolite and/or accumulation of the pathological protein; in some further alternative or additional embodiments (iii), at least one mammalian cell that display accumulation of the metabolite and/or accumulation of the pathological protein; and in some further alternative or additional embodiments (iv), at least one mammalian animal model that display accumulation of the metabolite and/or accumulation of the pathological protein. The next step (II), involves determining in the cells, and/or unicellular organism, and/or multicellular organism or mammal used in step (I), at least one phenotype associated with the accumulation of the metabolite and/or accumulation of the pathological protein. The next step (III), involves determining that the candidate is a therapeutic compound for the protein misfolding disorder if a change or modulation of at least one of the following parameters is observed in treated as compared to untreated cells and/or organisms of the at least one of the systems or platforms used for further validation or evaluation. Thus, a modulation and/or change in at least one of: the accumulation of the metabolite and/or accumulation of the pathological protein, and/or the observed phenotype, in the unicellular and/or multicellular organisms used and treated with the candidate compound as compared with the level of at least one of, the accumulation of the metabolite, and/or accumulation of the pathological protein, and/or the phenotype, in the unicellular and/or multicellular organisms, in the absence of the candidate compound.

In some further embodiments, the phenotype associated with accumulation of the metabolite and/or misfolding of the pathological protein is at least one of cell toxicity and formation of metabolite aggregate.

In some specific embodiments of the methods disclosed herein, the cell toxicity is determined by measuring at least one of cell viability, cell proliferation, cell apoptosis and any toxic phenotype on the organism or cell used in the screening and/or further evaluation and validation as discussed above. In some embodiments, at least one of, the accumulation of the metabolite, the formation of metabolite aggregation and/or misfolding of the pathological protein is determined by metabolic profiling, and/or microscopy, and/or light diffraction, and/or absorption, and/or scattering assay, and/or spectrometric assay, and/or immunological assay, and/or flow cytometry, and/or Liquid Chromatography, and/or NMR and stereoscopy.

In more specific embodiments, the metabolite is any one of an amino acid residue, a nucleobase, nucleoside, nucleotide, carbohydrate, fatty acid and ketone, sterols, porphyrin and haem, lipid, sphingolipid, phospholipid and lipoprotein, neurotransmitters, vitamins and (non-protein) cofactors, pterin, trace elements, metals, metabolites associated with energy metabolism, metabolites associated with peroxisome functions, or any intermediate product, derivative or metabolite thereof.

Still further, in some embodiments of the methods disclosed herein, the metabolite is at least one amino acid residue, any derivative, or any intermediate product or metabolite thereof.

In some specific embodiments, the amino acid residue or any intermediate product or metabolite thereof is at least one of: Homocysteine, Phenylalanine. Tyrosine, Glycine, Arginine, Cysteine, Isoleucine, Leucine, Lysine, Methionine, Proline, Tryptophane, Valine, N-acetylaspartate (NAA), Homogentisic acid, and any derivatives thereof.

In more specific embodiments, the amino acid residue or any intermediate product or metabolite thereof is Homocysteine. In such case, the yeast cell and/or yeast cell line, and/or yeast cell population, used by methods disclosed herein may carry a genetic and/or epigenetic modification in the Cystathionine beta-synthase 4 (CYS4) yeast gene.

Still further, in some embodiments, the genetically and/or epigenetically modified yeast cell and/or yeast cell line, and/or yeast cell population, display reduced or no expression of CYS4 gene, thereby displaying accumulation of Homocysteine and any derivative thereof.

In yet some alternative embodiments, the amino acid residue or any intermediate product or metabolite thereof is Homocysteine, and the yeast cell and/or yeast cell line, and/or yeast cell population used by the methods disclosed herein, grow under conditions of Homocysteine supplementation.

In some embodiments, the disclosed methods are applicable for screening for therapeutic compounds that may be suitable for any proteinopathy, protein misfolding and/or protein aggregation disorder. In some specific and non-limiting embodiments, such disorder may be any neurodegenerative disorder or any early signs or symptoms associated therewith.

Still further, in some embodiments, the compound identified by the screening methods of the present disclosure may be applicable for neurodegenerative disorder. In some embodiments, such disorder is characterized by at least one of beta-amyloid protein aggregation, an alpha synuclein beta-synuclein aggregates and tau protein aggregation.

In some embodiments, pathological protein is at least one of beta-amyloid protein, alpha synuclein and/or beta-synuclein and tau protein.

In yet some further embodiments, the methods disclosed herein may be used for screening for therapeutic compounds that may be applicable for the treatment of beta-amyloid protein aggregation disorder or tauopathy, specifically, at least one of Alzheimer's disease (AD), and age-associated cognitive decline (ACD). Still further the methods disclosed herein may be used for screening for therapeutic compounds that may be applicable for the treatment of alpha-synuclein pathology, for example, at least one of Parkinson disease (PD). Dementia with Lewy Bodies (DLB) and multiple system atrophy (MSA). In yet some further embodiments, the methods disclosed herein may be used for screening for therapeutic compounds that may be applicable for the treatment of at least one TAR DNA-binding protein 43 pathology, specifically, amyotrophic lateral sclerosis (ALS). In certain embodiments, the methods disclosed herein may be used for screening for therapeutic compounds that may be applicable for the treatment of huntingtin protein pathology, specifically, Huntington disease. In some further embodiments, the methods disclosed herein may be used for screening for therapeutic compounds that may be applicable for the treatment of islet amyloid polypeptide pathology, specifically. Type 2 diabetes. In some further embodiments, the methods disclosed herein may be used for screening for therapeutic compounds that may be applicable for the treatment of serum amyloid protein pathology, specifically, systemic amyloidosis.

A further aspect of the present disclosure relates to a screening method for candidate therapeutic compounds for treating, preventing, ameliorating, reducing or delaying the onset of a disorder characterized by at least one of beta-amyloid protein aggregation. In some embodiments, the method comprising the steps of; First step (a), involves contacting a yeast cell and/or yeast cell line, and/or yeast cell population, that display accumulation of Homocysteine, with a candidate compound.

More specifically, in some embodiments, the cells used are characterized by at least one of: in some embodiments (i), the yeast cell/s carry at least one manipulation in at least one yeast metabolic pathway that leads to accumulation of the homocysteine (Hcy). In some additional or alternative embodiments (ii), the yeast cell/s grow under conditions that result in accumulation of the Hcy. In some additional or alternative embodiments (iii), the yeast cell/s endogenously and/or exogenously express the beta-amyloid protein or any pathological protein associated with the disorder.

The next step (b), involves determining in the contacted cells obtained in step (a), at least one of the following parameters or indications: the accumulation of the Hcy, and/or beta-amyloid protein aggregation and/or accumulation, and/or at least one phenotype associated with the accumulation of the at least one of Hcy and any derivative thereof and/or beta-amyloid protein. Still further, the next step (c), involves determining that the candidate is a therapeutic compound, at least a potential therapeutic compound (that may be in some embodiments further evaluated and validated) for the disorder characterized by at least one of beta-amyloid protein aggregation if a change and/or modulation is detected or observed in cells treated by the candidate compound, in at least one of, the accumulation of said Hcy, and/or the beta-amyloid protein aggregation and/or accumulation, and/or the phenotype, as compared with the accumulation of the Hcy and/or said beta-amyloid protein aggregation and/or accumulation, and/or the phenotype in the absence of the candidate compound, specifically, in untreated cells or cells that were treated with a control compound.

As indicated above, the method of the invention is intended for screening for candidate compounds that may be useful in treating protein misfolding disorders associated with aggregation of at least one metabolite. In more specific embodiments, such metabolite may be any one of an amino acid residue, a nucleobase, carbohydrate, fatty acid and ketone, sterols, porphyrin and haem, lipid and lipoprotein, neurotransmitters, vitamins and (non-protein) cofactors, trace elements, metals, metabolites associated with energy metabolism, metabolites associated with peroxisome functions, or any intermediate product, derivative or metabolite thereof.

It should be appreciated that in some embodiments, screening methods in accordance with the invention may be a high throughput screen (HTS). High throughput screens often involve testing large numbers of compounds with high efficiency, e.g., in parallel. For example, tens or hundreds of thousands of compounds can be routinely screened in short periods of time, e.g., hours to days. Often such screening is performed in multi-well plates containing, e.g., e.g., 96, 384, 1536, 3456, or more wells (sometimes referred to as micro-well or microtiter plates or dishes) or other vessels in which multiple physically separated cavities or depressions or areas are present in or on a substrate. High throughput screens can involve use of automation, e.g., for liquid handling, imaging, data acquisition and processing, etc. Certain general principles and techniques that may be applied in embodiments of a HTS are known in the art, for example, as described in Macarron R & Hertzberg R P. Design and implementation of high-throughput screening assays. Methods Mol Biol., 565: 1-32, 2009.

A further aspect of the present disclosure provides a method for treating, preventing, ameliorating, reducing or delaying the onset of at least one protein misfolding disorder.

More specifically, the method disclosed herein comprises the following steps:

First step (I), obtaining a compound that modulates the level of at least one phenotype associated with the accumulation of at least one metabolite by a screening method. In some particular embodiments, the screening method comprises:

First (a), contacting a manipulated and/or modified yeast cell and/or yeast cell line, and/or yeast cell population that display accumulation of at least one metabolite, with a candidate compound. The modified and/or manipulated cells used in the disclosed methods may be further characterized by at least one of: in some embodiments (i), the yeast cell/s carry at least one manipulation and/or modification in at least one yeast metabolic pathway that leads to accumulation of the metabolite. In some additional and/or alternative embodiments (ii), the yeast cell/s grow under conditions that result in accumulation of said metabolite. In some additional and/or alternative embodiments (iii), the yeast cell/s endogenously and/or exogenously express at least one pathological protein associated with the proteinopathy and/or protein misfolding disorder. Next in step (b), determining in the contacted cells obtained in step (a), at least one of: (i) the accumulation of the metabolite; (iii) accumulation of the pathologic protein; (iii) the level of at least one phenotype associated with the accumulation of the metabolite and/or accumulation of the pathological protein. The next step (c), involves determining that the candidate is a therapeutic compound for the protein misfolding disorder if a change and/or modulation is observed when cells treated with the candidate compound are compared with untreated cells (or cells not treated with the candidate compound). The following at least one parameters are compared: the accumulation of the metabolite and/or accumulation of the pathological protein and/or the phenotype, is modulated as compared with the accumulation of the metabolite, and/or misfolding of the pathological protein and/or the phenotype in the absence vs. the presence of the candidate compound. The next step of the therapeutic methods disclosed herein involves (II), administering a therapeutic effective amount of the compound obtained by step (I), to a subject suffering from the at least one protein misfolding disorder.

In some embodiments, the compound useful in the therapeutic methods disclosed herein is obtained by a screening method as defined by the present disclosure.

The present disclosure further provides therapeutic methods for treating proteinopathies and/or protein misfolding disorders. As used herein, “disease”, “disorder”, “condition” and the like, as they relate to a subject's health, are used interchangeably and have meanings ascribed to each and all of such terms. It should be appreciated that the invention provides therapeutic methods applicable for any of the disorders disclosed above, as well as to any condition or disease associated therewith. It is understood that the interchangeably used terms “associated”, “linked” and “related”, when referring to pathologies herein, and in some embodiments refers to the metabolite associated with the proteinopathies and/or protein misfolding disorder, mean diseases, disorders, conditions, or any pathologies which at least one of: share causalities, co-exist at a higher than coincidental frequency, or where at least one accumulated metabolite share causalities, co-exist at a higher than coincidental frequency with at least one disorder, specifically, proteinopathies and/or protein misfolding disorder, or where at least one disease, disorder condition or pathology causes the second disease, disorder, condition or pathology. More specifically, as used herein, “disease”, “disorder”, “condition”, “pathology” and the like, as they relate to a subject's health, are used interchangeably and have meanings ascribed to each and all of such terms.

The terms “treat, treating, treatment” as used herein and in the claims mean ameliorating one or more clinical indicia of disease activity by administering a pharmaceutical composition of the invention in a patient having a pathologic disorder.

The term “treatment” as used herein refers to the administering of a therapeutic amount of the compounds obtained by the screening systems and methods provided by the invention, or any composition thereof which is effective to ameliorate undesired symptoms associated with a disease, to prevent the manifestation of such symptoms before they occur, to slow down the progression of the disease, slow down the deterioration of symptoms, to enhance the onset of remission period, slow down the irreversible damage caused in the progressive chronic stage of the disease, to delay the onset of said progressive stage, to lessen the severity or cure the disease, to improve survival rate or more rapid recovery, or to prevent the disease form occurring or a combination of two or more of the above.

The term “prevention” as used herein, includes the prevention or postponement of development of the disease, prevention or postponement of development of symptoms and/or a reduction in the severity of such symptoms that will or are expected to develop, preventing the occurrence or reoccurrence of the acute disease attacks. These further include ameliorating existing symptoms, preventing-additional symptoms and ameliorating or preventing the underlying metabolic causes of symptoms.

The term “amelioration” as referred to herein, relates to a decrease in the symptoms, and improvement in a subject's condition brought about by the compositions and methods according to the invention, wherein said improvement may be manifested in the forms of inhibition of pathologic processes associated with the protein misfolding disorders described herein, a significant reduction in their magnitude, or an improvement in a diseased subject physiological state.

The term “inhibit” and all variations of this term is intended to encompass the restriction or prohibition of the progress and exacerbation of pathologic symptoms or a pathologic process progress, said pathologic process symptoms or process are associated with.

The term “eliminate” relates to the substantial eradication or removal of the pathologic symptoms and possibly pathologic etiology, optionally, according to the methods of the invention described below.

The terms “delay”, “delaying the onset”, “retard” and all variations thereof are intended to encompass the slowing of the progress and/or exacerbation of a pathologic disorder or an infectious disease and their symptoms slowing their progress, further exacerbation or development, so as to appear later than in the absence of the treatment according to the invention. More specifically, treatment or prevention include the prevention or postponement of development of the disease, prevention or postponement of development of symptoms and/or a reduction in the severity of such symptoms that will or are expected to develop. These further include ameliorating existing symptoms, preventing-additional symptoms and ameliorating or preventing the underlying metabolic causes of symptoms.

It should be appreciated that the terms “inhibition”, “moderation”, “reduction” or “attenuation” as referred to herein, relate to the retardation, restraining or reduction of a process by any one of about 1% to 99.9%, specifically, about 1% to about 5%, about 5% to 10%, about 10% to 15%, about 15% to 20%, about 20% to 25%, about 25% to 30%, about 30% to 35%, about 35% to 40%, about 40% to 45%, about 45% to 50%, about 50% to 55%, about 55% to 60%, about 60% to 65%, about 65% to 70%, about 75% to 80%, about 80% to 85% about 85% to 90%, about 90% to 95%, about 95% to 99%, or about 99% to 99.9%. With regards to the above, it is to be understood that, where provided, percentage values such as, for example, 10%, 50%, 120%, 500%, etc., are interchangeable with “fold change” values, i.e., 0.1, 0.5, 1.2, 5, etc., respectively.

The present invention relates to the treatment of subjects, or patients, in need thereof. By “patient” or “subject in need” it is meant any organism to whom the preventive and prophylactic combinations, composition/s, kit/s, and methods herein described is desired, including humans and domestic mammals. In some specific embodiments, the treated subject may be a human subject. The subject may be male or female, a child or an adult. In exemplary embodiments, the subject is an adult (e.g., at least 18 years old). The present invention relates to the treatment of subjects, or patients, in need thereof. It should be further noted that particularly in case of human subject, administering of the compositions of the invention to the patient includes both self-administration and administration to the patient by another person.

The terms “effective amount” or “sufficient amount” mean an amount necessary to achieve a selected result. The “effective treatment amount” is determined by the severity of the disease in conjunction with the preventive or therapeutic objectives, the route of administration and the patient's general condition (age, sex, weight and other considerations known to the attending physician).

A further aspect of the present disclosure relates to a therapeutic compound for treating, preventing, ameliorating, reducing or delaying the onset of at least one protein misfolding disorder. In some embodiments, the compound is identified by a method comprising the steps of; first, in step (a), contacting a manipulated yeast cell and/or yeast cell line, and/or yeast cell population that display accumulation of at least one metabolite, with a candidate compound, wherein at least one of: (i) the yeast cell/s carry at least one manipulation in at least one yeast metabolic pathway that leads to accumulation of the metabolite, (ii) the yeast cell/s grow under conditions that result in accumulation of the metabolite, (iii) the yeast cell/s endogenously and/or exogenously express at least one pathological protein associated with the protein misfolding disorder. In the next step (b), determining in the contacted cells obtained in step (a), at least one of: the accumulation of the metabolite and/or the pathologic protein, and/or the level of at least one phenotype associated with the accumulation of the metabolite and/or accumulation of the pathological protein. In the next step (c), determining that the candidate is a therapeutic compound for the proteinopathy and/or the protein misfolding disorder if a change or modulation is observed in cells treated by the examined candidate compound, in at least one of the following parameters: the accumulation of the metabolite and/or accumulation of the pathological protein and/or the phenotype is modulated as compared with the accumulation of the metabolite, and/or accumulation of the pathological protein and/or said phenotype in the absence of the candidate compound. In some embodiments, the compound is identified by any of the methods defined by the present disclosure.

In more specific embodiments, the compound may be at least one of a small molecule/s, aptamer, a peptide, a nucleic acid molecule and an immunological agent, and any combinations thereof.

Still further, in some embodiments, the present invention provides therapeutic compounds based on screening of candidate compounds using the systems and methods provided by the invention.

A “Compound” is used herein to refer to any substance, agent (e.g., molecule), supramolecular complex, material, or combination or mixture thereof. A compound may be any agent that can be represented by a chemical formula, chemical structure, or sequence. Example of compounds applicable for the present invention, include, e.g., small molecules, polypeptides, nucleic acids (e.g., RNAi agents, antisense oligonucleotide, aptamers), lipids, polysaccharides, etc. In general, compounds may be obtained using any suitable method known in the art. The ordinary skilled artisan will select an appropriate method based, e.g., on the nature of the compound. A compound may be at least partly purified. In some embodiments a compound may be provided as part of a composition, which may contain, e.g., a counter-ion, aqueous or non-aqueous diluent or carrier, buffer, preservative, or other ingredient, in addition to the compound, in various embodiments. In some embodiments a compound may be provided as a salt, ester, hydrate, or solvate. In some embodiments a compound is cell-permeable, e.g., within the range of typical compounds that are taken up by cells and acts intracellularly, e.g., within mammalian cells, to produce a biological effect. Certain compounds may exist in particular geometric or stereoisomeric forms. Such compounds, including cis- and trans-isomers, E- and Z-isomers, R- and S-enantiomers, diastereomers. (D)-isomers, (L)-isomers, (−)- and (+)-isomers, racemic mixtures thereof, and other mixtures thereof are encompassed by this disclosure in various embodiments unless otherwise indicated. Certain compounds may exist in a variety or protonation states, may have a variety of configurations, may exist as solvates (e.g., with water (i.e., hydrates) or common solvents) and/or may have different crystalline forms (e.g., polymorphs) or different tautomeric forms. Embodiments exhibiting such alternative protonation states, configurations, solvates, and forms are encompassed by the present disclosure where applicable. Still further, in certain embodiments, candidate compounds can be screened from large libraries of synthetic or natural compounds. A compound to be tested may be referred to as a test compound or a candidate compound. Any compound may be used as a test compound in various embodiments. In some embodiments a library of FDA approved compounds that can be used by humans may be used. Compound libraries are commercially available from a number of companies including but not limited to Maybridge Chemical Co. (Trevillet, Cornwall, UK), Comgenex (Princeton, N.J.), Microsource (New Milford, Conn.), Aldrich (Milwaukee, Wis.), AKos Consulting and Solutions GmbH (Basel, Switzerland), Ambinter (Paris, France), Asinex (Moscow, Russia), Aurora (Graz, Austria), BioFocus DPI, Switzerland, Bionet (Camelford, UK), ChemBridge, (San Diego, Calif.), ChemDiv, (San Diego, Calif.), Chemical Block Lt, (Moscow, Russia). ChemStar (Moscow, Russia), Exclusive Chemistry. Ltd (Obninsk, Russia), Enamine (Kiev, Ukraine), Evotec (Hamburg, Germany), Indofine (Hillsborough, N.J.), Interbio screen (Moscow, Russia), Interchim (Montlucon, France), Life Chemicals, Inc. (Orange, Conn.), Microchemistry Ltd. (Moscow, Russia), Otava, (Toronto, ON), PharmEx Ltd. (Moscow, Russia), Princeton Biomolecular (Monmouth Junction, N.J.), Scientific Exchange (Center Ossipee, N.H.), Specs (Delft, Netherlands), TimTec (Newark, Del.), Toronto Research Corp. (North York ON). UkrOrgSynthesis (Kiev. Ukraine), Vitas-M, (Moscow, Russia), Zelinsky Institute, (Moscow, Russia), and Bicoll (Shanghai, China). Combinatorial libraries are available and can be prepared. Libraries of natural compounds in the form of bacterial, fungal, plant and animal extracts are commercially available or can be readily prepared by methods well known in the art. Compounds isolated from natural sources, such as animals, bacteria, fungi, plant sources, and marine samples may be tested for the presence of potentially useful pharmaceutical compounds. It will be understood that the agents to be screened could also be derived or synthesized from chemical compositions or man-made compounds. In some embodiments a library useful in the present invention may comprise at least 10,000 compounds, at least 50,000 compounds, at least 100,000 compounds, at least 250,000 compounds, or more.

In some specific embodiments, the compound of the invention may be a small molecule. A “small molecule” as used herein, is an organic molecule that is less than about 2 kilodaltons (kDa) in mass. In some embodiments, the small molecule is less than about 1.5 kDa, or less than about 1 kDa. In some embodiments, the small molecule is less than about 800 daltons (Da), 600 Da, 500 Da, 400 Da, 300 Da, 200 Da, or 100 Da. Often, a small molecule has a mass of at least 50 Da. In some embodiments, a small molecule is non-polymeric. In some embodiments, a small molecule is not an amino acid. In some embodiments, a small molecule is not a nucleotide. In some embodiments, a small molecule is not a saccharide. In some embodiments, a small molecule contains multiple carbon-carbon bonds and can comprise one or more heteroatoms and/or one or more functional groups important for structural interaction with proteins (e.g., hydrogen bonding), e.g., an amine, carbonyl, hydroxyl, or carboxyl group, and in some embodiments at least two functional groups. Small molecules often comprise one or more cyclic carbon or heterocyclic structures and/or aromatic or polyaromatic structures, optionally substituted with one or more of the above functional groups. It should be understood that the candidate compound used in the systems and methods of the invention may be any of the compounds as described herein above.

It should be understood That the therapeutic methods and uses disclosed herein may be applicable for any of the protein misfolding disorders disclosed by the present disclosure. Still further aspect of the present disclosure relates to a method for the detection, and/or monitoring of at least one protein misfolding disorder or proteniopathy in a subject. The diagnostic method of the present disclosure comprises: first (a), contacting at least one biological sample of the subject with at least one antibody specific for at least one metabolite-fibrils. It should be appreciated that the sample, or any aliquot or part thereof, may be alternatively contacted with any other affinity molecule that specifically recognizes and binds metabolite-fibril/s. The next step (b), involves classifying the subject as a subject affected by the at least one protein misfolding disorder or proteniopathy, if the metabolite-fibril is detected in the sample. In some embodiments, the diagnostic method may further comprise an optional additional therapeutic step (c), involving administering to a subject classified as affected by the at least one protein misfolding disorder and/or proteniopathy, a therapeutically effective amount of at least one anti-proteinopathy or an anti-protein misfolding disorder therapeutic agent.

In some specific embodiments, the diagnostic method is specifically applicable for early diagnosing and/or monitoring disorders associated with accumulation of amyloid beta, specifically. Alzheimers disease (AD). According to some specific embodiments, the anti-Hcy-fibril antibodies of the present disclosure are used. Detection of the Hcy-fibril in the examined sample indicates that the subject is affected by a disorder associated with accumulation of amyloid beta. In some embodiments, such disorders include, but are not limited to any of the disclosed proteinopathies, specifically, AD. Thus, in yet some further embodiments, the antibodies used may be antibodies, or any other affinity molecule that recognize and bind Hcy-fibril/s.

The present disclosure further provides a diagnostic kit for the diagnosis and monitoring of proteinopathies and/or protein misfolding disorders in a subject. The disclosed kit comprises any of the metabolite-fibril specific antibodies. As disclosed herein after in connection with the diagnostic methods and kits of the present disclosure, the kit of the invention may comprise the disclosed anti-metabolite-fibril/s antibodies, that may be provided in the disclosed kit/s either in solution, or in dried form, attached and/or non attached to a solid support as discussed herein after, disclosed antibodies may be provided in an array or any device (for example lateral flow device) appropriate for performing the immunological recognition of the metabolite-fibril/s by the disclosed antibodies.

As shown by the preset disclosure, the formation of metabolite fibrils, for example, Hcy fibrils is an initiating step for the formation of the pathological protein assemblies and aggregates, for example, the beta-amyloid aggregates and/or assemblies. Therefore, in some embodiments, the disclosed diagnostic methods and kits may be used for early detection of the discussed proteinopathies in subjects. Specifically, for detecting the initiating step of the assemblies of the pathogenic proteins that eventually lead to the proteinopathy. Thus, the diagnostic methods of the preset disclosure provide a powerful means for detecting an “early diagnosis” or “early detection” may be used interchangeably, and provides diagnosis prior to appearance of clinical symptoms. Prior as used herein is meant days, weeks, months or even years before the appearance of such symptoms. More specifically, at least 1 week, at least 1 month, 2 months, 3 months, 4 months, 5 months, 6 months, 7 months, 8 months, 9 months, 10 months, 11 months, 12 months, or even few years before clinical symptoms appear. As indicated above, the invention provides diagnostic and prognostic methods. “Prognosis” is defined as a forecast of the future course of a disease or disorder, based on medical knowledge. This highlights the major advantage of the invention, namely, the ability to predict progression of the disease, based on the detection of metabolite-fibril/s, that ae the initiating step in aggregation and fibril formation of the pathogenic proteins that are known to be associated with the protein misfolding disorders disclosed herein.

In some embodiments of the diagnostic methods and kits disclosed herein, the metabolite-fibril/s existence or level/s are detected upon contacting the sample with the antibody, and thereby enabling the classification step. Thus, the presence of the metabolite fibrils is detected and the levels in the examined sample are determined and generally considered for classification purpose as “positive” or “negative”. Detected metabolite-fibril/s level values that are higher (positive) or lower (negative) in comparison with a corresponding predetermined standard metabolite-fibril value or a cut-off value in a control sample predict to which population of subjects, either healthy or diseased, the tested sample belongs, and in some embodiments, may even reflect the disease stage, or the protein aggregation status of the subject. Specifically, the aggregation of the pathologic misfolded protein involved and associated with the specific proteinopathy).

It should be appreciated that an important step in the method of the inventions is determining whether the detected value of any one of the metabolite-fibril/s is changed or different when compared to a pre-determined cut off or is within the range of metabolite-fibril/s levels of such cutoff. Alternatively, or in addition, the metabolite-fibril/s value determined may be compared to the metabolite-fibril/s value of a control sample, for example, a sample obtained from a healthy subject or from a subject that is not affected by the specific proteinopathy and/or protein misfolding disorder, for example, AD.

Thus, in yet more specific embodiments, the second step (b) of the method of the invention further involves comparing the metabolite-fibril/s values determined for the tested sample with predetermined standard values or cutoff values, or alternatively, with metabolite-fibril/s values of at least one control sample. As used herein the term “comparing” denotes any examination of the metabolite-fibril/s level and/or metabolite-fibril/s values obtained in the samples of the invention as detailed throughout in order to discover similarities or differences between at least two different samples. It should be noted that in some embodiments, comparing according to the present invention encompasses the possibility to use a computer-based approach. As described hereinabove, the method of the invention refers to a predetermined cutoff value/s. It should be noted that a “cutoff value”, sometimes referred to simply as “cutoff” herein, is a value that meets the requirements for both high diagnostic sensitivity (true positive rate) and high diagnostic specificity (true negative rate). The terms “sensitivity” and “specificity” are used herein with respect to the ability of one or more anti-metabolite-fibril/s antibodies, to correctly classify a sample as belonging to a pre-established population associated with the specific proteinopathy and/or protein misfolding disorder, for example, AD, or alternatively, to a pre-established population of healthy subjects or subjects that are not affected by AD. In other words, to correctly classify a sample as a sample of a subject affected by specific proteinopathy and/or protein misfolding disorder, for example, AD or alternatively as a subject that is not affected by the specific proteinopathy and/or protein misfolding disorder, for example, AD (either healthy or not). “Sensitivity” indicates the performance of the disclosed anti-metabolite-fibril/s antibodies of the invention, with respect to correctly classifying samples as belonging to pre-established populations that are likely to suffer from a disease or disorder or characterized at different stages of a disease, if the metabolite-fibril/s are formed in such subject. “Specificity” indicates the performance of the anti-metabolite-fibril/s antibodies of the invention with respect to correctly classifying and distinguishing between samples as belonging to pre-established populations of subjects suffering from the same disorder and populations of subjects that are either healthy or not affected by the specific proteinopathy and/or protein misfolding disorder, for example, AD.

Simply put, “sensitivity” relates to the rate of identification of the patients (samples) as such out of a group of samples, whereas “specificity” relates to the rate of correct identification of the specific proteinopathy and/or protein misfolding disorder, for example, AD samples as such out of a group of samples. Cutoff values may be used as control sample/s or in addition to control sample/s, the cutoff values being the result of a statistical analysis of metabolite-fibril/s value/s and/or levels (specifically, the Hcy-fibril/s of the invention) differences in pre-established populations healthy or suffering from specific proteinopathy and/or protein misfolding disorder, for example, AD. Pre-established populations as used herein refer to populations of patients diagnosed with the specific proteinopathy and/or protein misfolding disorder, for example, AD (by any conventional means), or alternatively, populations of healthy subjects.

In yet some further embodiments, a negative or positive determination of the metabolite-fibril/s levels and/or value as compared to the predetermined cutoff values, or the metabolite-fibril/s value of a control sample, also encompass values that are within the range of the discussed cutoff. More specifically, in case the particular metabolite-fibril/s is found to be present and formed in the specific proteinopathy and/or protein misfolding disorder, for example, AD, a metabolite-fibril/s value that is determined by the method of the invention as “positive” when compared to a predetermined cutoff of population of patients suffering from the specific proteinopathy and/or protein misfolding disorder, for example, AD, or to the metabolite-fibril/s value of at least one, and preferably, more, known patient/s suffering from specific proteinopathy and/or protein misfolding disorder, for example, AD. This may indicate that the examined subject belongs to a population suffering from specific proteinopathy and/or protein misfolding disorder, for example, AD (e.g., that the subject carries or is affected by specific proteinopathy and/or protein misfolding disorder, for example, AD), in case that the metabolite-fibril/s value is either higher (positive) or fall within the range (the average values of the cutoff predetermined for patient population suffering from specific proteinopathy and/or protein misfolding disorder, for example, AD) of the control or standard value. Ina similar manner, a subject exhibiting a metabolite-fibril/s value that is “negative” (or not existing) as compared to the cutoff patients, may be considered as belonging to population that is not suffering from specific proteinopathy and/or protein misfolding disorder, for example, AD. In some embodiments, “fall within the range” encompass values that differ from the cutoff value in about 1%, about 5%, about 10%, about 15%, about 20%, about 25%, about 30%, about 35%, about 40%, about 45%, about 50% or more. Simply put, a “positive” metabolite-fibril/s value as used herein refers to high metabolite-fibril/s value that reflects existence of metabolite-fibril/s, fibril formation and even in some embodiments, moderate metabolite-fibril/s value. A “negative” metabolite-fibril/s value reflects a repressed, low, reduced, or non-existing metabolite-fibril/s (lack of metabolite-fibril/s formation). Thus, in some embodiments, when a specific biomarker is overexpressed in specific proteinopathy and/or protein misfolding disorder, for example, AD, a “positive” metabolite-fibril/s value of an examined sample may be a value that is higher or within the range of the metabolite-fibril/s value of a sample taken from a patient affected with specific proteinopathy and/or protein misfolding disorder, for example, AD, or a standard cutoff value calculated for specific proteinopathy and/or protein misfolding disorder, for example, AD patients. A “negative” value would be a metabolite-fibril/s value that is lower than the metabolite-fibril/s value of the specific proteinopathy and/or protein misfolding disorder, for example, AD patients (or standard value, or the value of a control sample). Such value may be within the range of the value of a healthy control sample or a standard value of a healthy population of subject, or of subjects that are not affected by specific proteinopathy and/or protein misfolding disorder, for example, AD.

It should be appreciated that a “control sample” as used herein may reflect a sample of at least one subject (either healthy, a subject that is not affected by specific proteinopathy and/or protein misfolding disorder, for example, AD, or alternatively, an specific proteinopathy and/or protein misfolding disorder, for example, AD patient), and preferably, a mixture at least two, at least three, at least four, at least five, at least six or more patients.

It should be emphasized that the nature of the invention is such that the accumulation of further patient data may improve the accuracy of the presently provided cutoff values, which are based on an ROC (Receiver Operating Characteristic) curve generated according to the patient data using analytical software program. The metabolite-fibril/s values are selected along the ROC curve for optimal combination of diagnostic sensitivity and diagnostic specificity which are as close to 100 percent as possible, and the resulting values are used as the cutoff values that distinguish between subjects who are diagnosed with the specific proteinopathy and/or protein misfolding disorder, for example, AD at a certain rate, and those who will not (with said given sensitivity and specificity). Similar analysis may be performed for example when diagnosis of specific proteinopathy and/or protein misfolding disorder, for example, AD is being examined to distingue between healthy tissue and neurodegenerative tissue. The ROC curve may evolve as more and more data and related metabolite-fibril/s values are recorded and taken into consideration, modifying the optimal cutoff values and improving sensitivity and specificity.

As noted above, the metabolite-fibril/s value determined for the examined sample (the normalized metabolite-fibril/s value) is compared with a predetermined cutoff or to a control sample. More specifically, in certain embodiments, the metabolite-fibril/s value obtained for the examined sample is compared with a predetermined standard or cutoff value.

In further embodiments, the predetermined standard metabolite-fibril/s value, or cutoff value has been pre-determined and calculated for a population comprising at least one of healthy subjects, subjects suffering from any disorder, subjects suffering from different stages of any disorder, subjects that respond to treatment, non-responder subjects, subjects in remission and subjects in relapse.

Still further, in certain alternative embodiments where a control sample is being used (instead of, or in addition to, pre-determined cutoff values), the metabolite-fibril/s value or the normalized metabolite-fibril/s values of the metabolite-fibril/s used by the invention in the test sample are compared to the metabolite-fibril/s values in the control sample. In certain embodiments, such control sample may be obtained from at least one of a healthy subject, a subject suffering from a disorder at a specific stage, a subject suffering from a disorder at a different specific stage a subject that responds to treatment, a non-responder subject.

It should be appreciated that “Standard” or a “predetermined standard” as used herein, denotes either a single standard value or a plurality of standards with which the level of at least one of the metabolite-fibril/s from the tested sample is compared. The standards may be provided, for example, in the form of discrete numeric values or is calorimetric in the form of a chart with different colors or shadings for different levels of metabolite-fibril/s; or they may be provided in the form of a comparative curve prepared on the basis of such standards (standard curve). In certain embodiments, the detection step further involves detecting a signal from the antibodies that correlates with the metabolite-fibril/s level of at least one of the metabolite-fibril/s in the sample from the subject, by a suitable means. According to some embodiments, the signal detected from the sample by any one of the experimental methods detailed herein below reflects the metabolite-fibril/s level of at least one of the metabolite-fibril/s. It should be noted that such signal-to-metabolite-fibril/s level data may be calculated and derived from a calibration curve.

Thus, in certain embodiments, the method of the invention may optionally further involve the use of a calibration curve created by detecting a signal for each one of increasing pre-determined concentrations of at least one of the metabolite-fibril/s. Obtaining such a calibration curve may be indicative to evaluate the range at which the detected levels correlate linearly with the concentrations of at least one of the metabolite-fibril/s. It should be noted in this connection that at times when no change in level of at least one of the metabolite-fibril/s is observed, the calibration curve should be evaluated in order to rule out the possibility that the measured metabolite-fibril/s level is not exhibiting a saturation type curve, namely a range at which increasing concentrations exhibit the same signal.

It should be noted that for determining the metabolite-fibril/s value/s of at least one of the metabolite-fibril/s of the invention, the methods of the invention may further comprise the step of providing at least one detecting molecule, specifically, an anti-metabolite-fibril/s antibody specific for determining the existence or levels of at least on of the metabolite-fibril/s described by the present disclosure. In some embodiments, such detecting molecules, specifically, anti-metabolite-fibril/s antibodies may be provided as a mixture, as a composition or as a kit. Thus, in some embodiments, the at least one anti-metabolite-fibril/s antibodies may be provided as a mixture of antibodies, wherein each antibody is specific for one metabolite-fibril/s. It should be appreciated however, that for each metabolite-fibril/s, one or several specific antibodies may be used and provided. In yet some further alternative embodiments, the antibodies may be provided separately for each metabolite-fibril/s, e.g., in specific tube, containers, slots, spots, wells, and the like. It further alternative embodiments, the antibodies may be attached or immobilized to a solid support, specifically, in recorded location.

In some embodiments, the kits and diagnostic methods provided herein comprise the anti-metabolite-fibril/s antibodies attached or immobilized or associated with a solid support.

As used herein, the term “immobilized” refers to a stable association of the anti-metabolite-fibril/s antibody with a surface of a solid support. By “stable association” is meant a physical association between two entities in which the mean half-life of association is one day or more, two days or more, one week or more, one month or more, including six months or more e.g., under physiological conditions. According to certain embodiments, the stable association arises from a covalent bond between the two entities, a non-covalent bond between the two entities (e.g., an ionic or metallic bond), or other forms of chemical attraction, such as hydrogen bonding, Van der Waals forces, and the like. Thus, in some embodiments, the anti-metabolite-fibril/s antibodies are attached to a solid support. Solid support suitable for use in the methods and kits of the present invention is typically substantially insoluble in liquid phases. Solid supports of the current invention are not limited to a specific type of support. Rather, a large number of supports are available and are known to one of ordinary skill in the art. Thus, useful solid supports include solid and semi-solid matrixes, such as aerogels and hydrogels, resins, beads, biochips (including thin film coated biochips), microfluidic chip, a silicon chip, nanoparticles, polymers, multi-well plates (also referred to as microtiter plates or microplates), membranes, filters, conducting and non-conducting metals, glass (including microscope slides) and magnetic supports. More specific examples of useful solid supports include, silica gels, polymeric membranes such as nitrocellulose, particles, derivatized plastic films, glass beads, cotton, plastic beads, alumina gels, polysaccharides such as Sepharose, nylon, latex bead, magnetic bead, paramagnetic bead, superparamagnetic bead, starch and the like. In yet some further embodiments, in case electrochemical assays are applied by the methods, devices and kits of the invention, solid support may further include nano- and micro-sized materials, such as gold nanoparticles (GNPs), carbon nanotubes (CNTs), graphene (GR), magnetic particles (MBs), quantum dots (QDs) and conductive polymers.

Still further, it should be noted that all steps for determining the different parameters indicated above, involve contacting the sample or any component thereof with a specific reagent (e.g., anti-metabolite-fibril/s antibodies).

The term “contacting” means to bring, put, incubates or mix together. As such, a first item is contacted with a second item when the two items are brought or put together, e.g., by touching them to each other or combining them. In the context of the present invention, the term “contacting” includes all measures or steps which allow interaction between the at least one of the antibodies of at least one of the metabolite-fibril/s, and optionally, for at least one suitable control of the tested sample. The contacting is performed in a manner so that the at least one of antibodies specific for the at least one of the metabolite-fibril/s for example, can interact with or bind to the at least one of the metabolite-fibril/s, in the tested sample. The binding will preferably be non-covalent, reversible binding, e.g., binding via salt bridges, hydrogen bonds, hydrophobic interactions or a combination thereof.

As indicated above, the disclosed antibodies specifically recognize and bind the specific metabolite-fibril/s, for example, the Hcy-fibril/s disclosed herein. It should be therefore noted that the term “binding specificity”, “specifically binds to an antigen”. “specifically immuno-reactive with”, “specifically directed against” or “specifically recognizes”, when referring to an epitope, specifically, a recognized epitope within the at least one of the metabolite-fibril/s, refers to a binding reaction which is determinative of the presence of the epitope in a heterogeneous population of fibrils and other metabolite/s. More particularly. “selectively bind” in the context of proteins encompassed by the invention refers to the specific interaction of an antibody with the specific metabolite-fibril/s, wherein the interaction preferentially occurs as between any two of antibody and the specific metabolite-fibril/s preferentially as compared with any other metabolite-fibril/s and antibody.

Thus, under designated immunoassay conditions, the specified antibodies bind to a particular epitope at least two times the background and more typically more than 10 to 100 times background. More specifically. “Selective binding”, as the term is used herein, means that a molecule binds its specific binding partner with at least 2-fold greater affinity, and preferably at least 10-fold, 20-fold, 50-fold, 100-fold or higher affinity than it binds a non-specific molecule.

It should be appreciated that the antibodies used by the methods of the invention, may be in some embodiments antibodies that are not naturally occurring antibodies. More specifically, the antibodies are not produced naturally in the body, and more specifically, it should be appreciated that production thereof involves immunological and recombinant techniques.

Still further, in some embodiments, when the anti-metabolite-fibril/s antibodies are used in the disclosed diagnostic methods and kits, the determination of the metabolite-fibril/s level of the metabolite-fibril/s may be performed by an immunological assay.

In some specific embodiments, determination of the level of the metabolite-fibril/s may be performed using ELISA. Enzyme-Linked Immunosorbent Assay (ELISA) is used herein involves fixation of a sample, potentially containing a metabolite-fibril/s to a surface such as a well of a microtiter plate. A metabolite-fibril/s-specific antibody coupled to an enzyme or other detectable moiety is applied and allowed to bind to the metabolite-fibril/s. Presence of the antibody is then detected and quantitated by a colorimetric reaction employing the enzyme coupled to the antibody. Enzymes commonly employed in this method include horseradish peroxidase and alkaline phosphatase. If well calibrated and within the linear range of response, the amount of metabolite-fibril/s present in the sample is proportional to the amount of color produced. A metabolite-fibril/s standard is generally employed to improve quantitative accuracy.

In some specific embodiments, different RI assays may be employed for determination of the level of the metabolite-fibril/s of the invention. In one version, Radioimmunoassay (RIA) involves precipitation of the desired target (i.e., the metabolite-fibril/s) with a specific antibody and radio labeled antibody-binding protein (e.g., protein A labeled with I¹²⁵) immobilized on a perceptible carrier such as agars beads. The radio-signal detected in the precipitated pellet is proportional to the amount of substrate bound.

Still further, in specific embodiments, determination of the metabolite-fibril/s level of the metabolite-fibril/s of the invention may be performed using FACS. Fluorescence-Activated Cell Sorting (FACS) involves detection of a target (e.g., the metabolite-fibril/s) in situ in cells bound by metabolite-fibril/s-specific, fluorescently labeled antibodies. The substrate-specific antibodies are linked to fluorophore. Detection is by means of a flow cytometry machine, which reads the wavelength of light emitted from each cell as it passes through a light beam. This method may employ two or more antibodies simultaneously and is a reliable and reproducible procedure used by the present invention. Immuno histochemical Analysis involves detection of a metabolite-fibril/s in situ in fixed cells by metabolite-fibril/s-specific antibodies. The metabolite-fibril/s specific antibodies may be enzyme-linked or linked to fluorophore or any other detectable moiety. Detection is by microscopy and is either subjective or by automatic evaluation. With enzyme-linked antibodies, a calorimetric reaction may be required. It will be appreciated that immunohistochemistry is often followed by counterstaining of the cell nuclei, using, for example, Hematoxyline or Giemsa stain.

As shown by the present disclosure, specific antibodies that recognize the metabolite-fibrils were prepared. In some embodiments, these antibodies were prepared against and are therefore specific for the Hcy-fibrils. The disclosed antibodies were prepared against a specific and well-defined antigen, specifically, a fibrillar form of a metabolite, for example, Hcy fibrils. As will be discussed herein after, the antigen used was extensively characterized, thereby allowing the creation of highly specific antibodies that recognize the fibril form of the metabolite, whereas commercial antibodies that recognize the metabolite itself, cannot recognize the fibril form. Preparation of the discussed antibodies is described in detail in the Experimental procedure section. The present disclosure therefore in some aspects thereof, relates, and therefore provides antibodies. The term “antibody” as used herein, means any antigen-binding molecule or molecular complex that specifically binds to or interacts with a particular antigen. The term “antibody” includes immunoglobulin molecules comprising four polypeptide chains, two heavy (H) chains and two light (L) chains inter-connected by disulfide bonds, as well as multimers thereof (e.g., IgM). Each heavy chain comprises a heavy chain variable region (abbreviated herein as HCVR or V_{H}) and a heavy chain constant region (CH). The heavy chain constant region comprises three domains. CH1, CH2 and CH3. Each light chain comprises a light chain variable region (abbreviated herein as LCVR or V_{L}) and a light chain constant region. The light chain constant region comprises one domain (CL1). The V_{H} and V_{L} regions can be further subdivided into regions of hypervariability, termed complementarity determining regions (CDRs), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4.

Typically, an antibody is composed of two immunoglobulin (Ig) heavy chains and two Ig light chains. In humans, antibodies are encoded by three independent gene loci, namely kappa (κ) chain (Igκ) and lambda (λ) chain (Igλ) genes for the Light chains and IgH genes for the Heavy chains, which are located on chromosome 2, chromosome 22, and chromosome 14, respectively.

The antibody used by the methods and kits of the invention may be any one of a polyclonal, a monoclonal or humanized antibody or any antigen-binding fragment thereof. As shown by the present disclosure, in some specific and non-limiting embodiments, any polyclonal antibodies, prepared as discussed herein, are useful in the disclosed methods and kits. The term “an antigen-binding fragment” refers to any portion of an antibody that retains binding to the antigen. Examples of antibody functional fragments include, but are not limited to, complete antibody molecules, antibody fragments, such as Fv, single chain Fv (scFv), complementarity determining regions (CDRs), V_{L} (light chain variable region), V_{H} (heavy chain variable region), Fab, F(ab)₂′ and any combination of those or any other functional portion of an immunoglobulin peptide capable of binding to target antigen, specifically, the metabolite-fibril/s, and more specifically, the Hcy fibril/s.

As appreciated by one of skill in the art, various antibody fragments can be obtained by a variety of methods, for example, digestion of an intact antibody with an enzyme, such as pepsin, or de novo synthesis. Antibody fragments are often synthesized de novo either chemically or by using recombinant DNA methodology. Thus, the term antibody, as used herein, includes antibody fragments either produced by the modification of whole antibodies, or those synthesized de novo using recombinant DNA methodologies (e.g., single chain Fv) or those identified using phage display libraries. The term antibody also includes bivalent molecules, diabodies, triabodies, and tetrabodies.

References to “V_{H}” or a “VH” refer to the variable region of an immunoglobulin heavy chain, including an Fv, scFv, a disulfide-stabilized Fv (dsFv) or Fab. References to “V_{L}” or a “VL” refer to the variable region of an immunoglobulin light chain, including of an Fv, scFv, dsFv or Fab.

More specifically, the phrase “single chain Fv” or “scFv” refers to an antibody in which the variable domains of the heavy chain and of the light chain of a traditional two chain antibody have been joined to form one chain. Typically, a linker peptide is inserted between the two chains to allow for the stabilization of the variable domains without interfering with the proper folding and creation of an active binding site. A single chain antibody applicable for the invention, e.g., may bind as a monomer. Other exemplary single chain antibodies may form diabodies, triabodies, and tetrabodies.

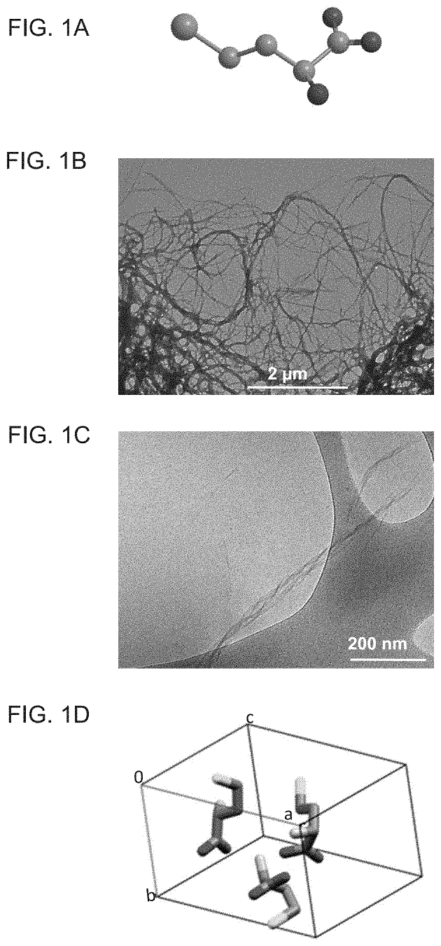

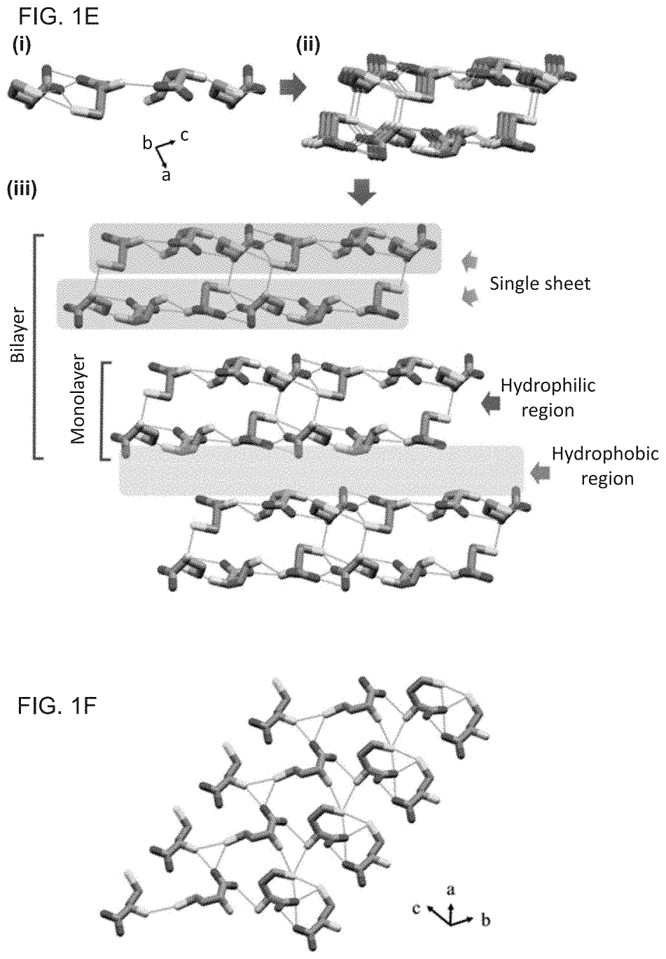

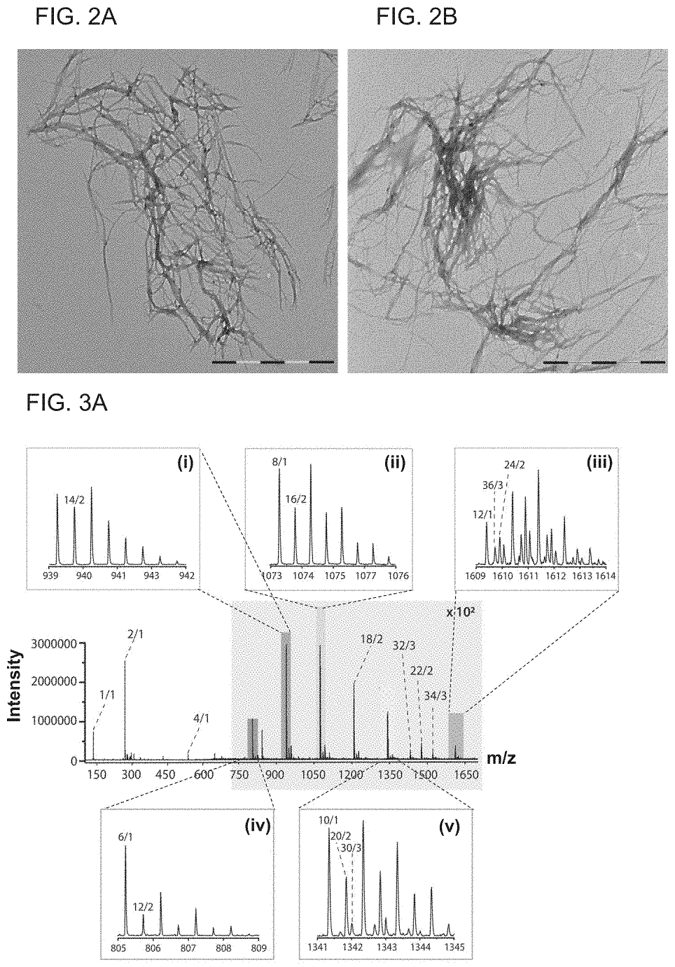

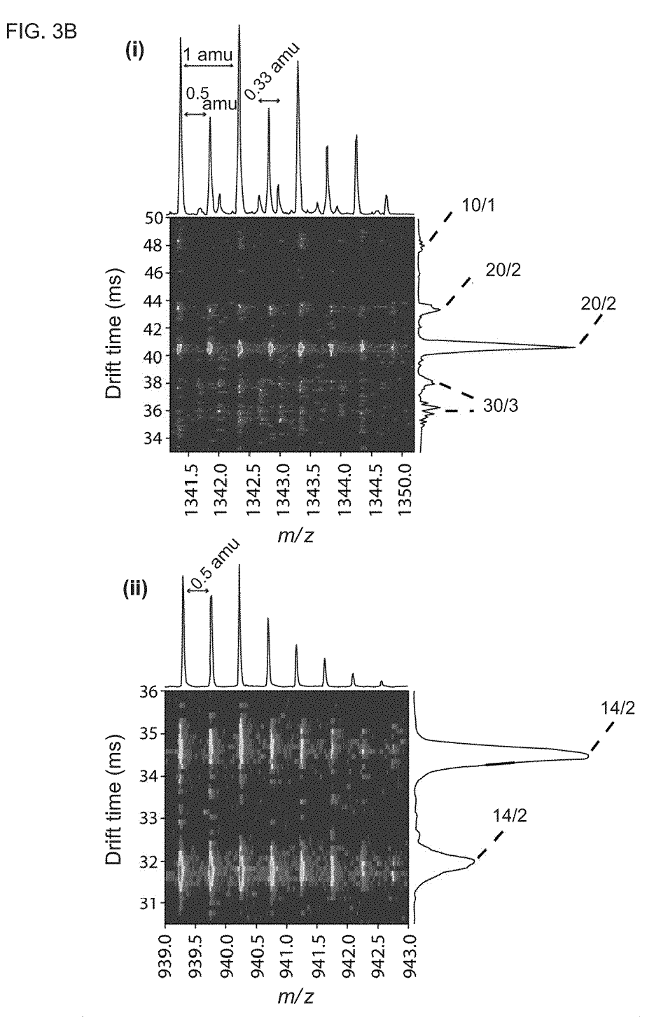

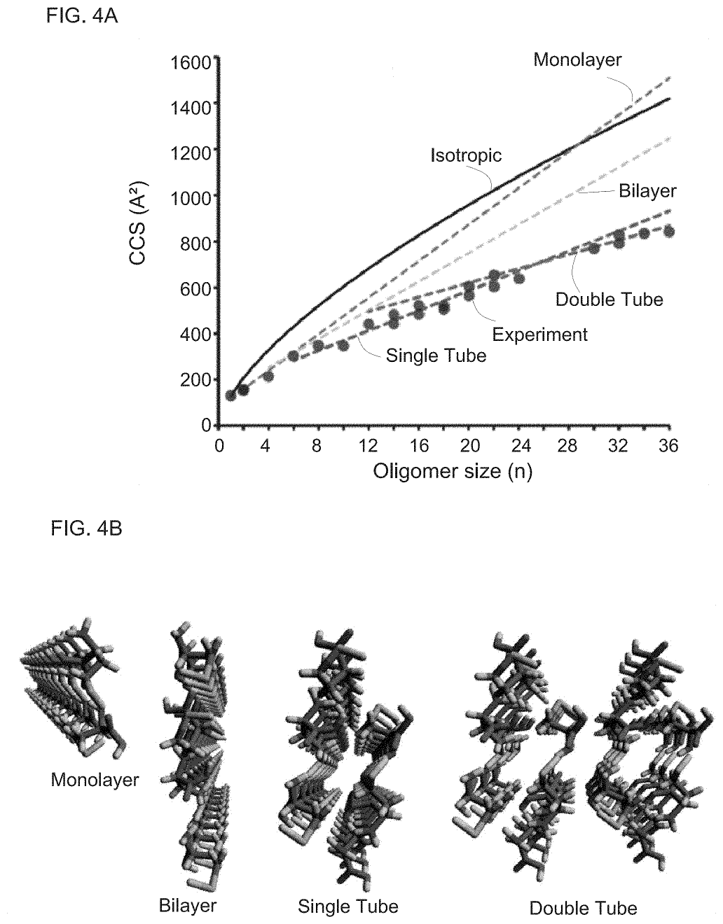

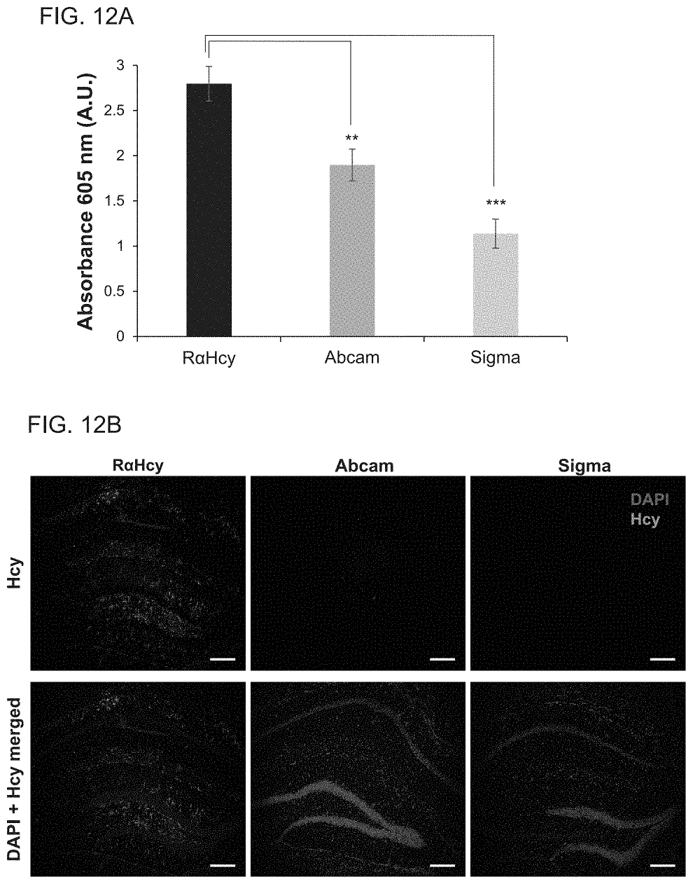

It should be appreciated that in some embodiments, any antibody used by the methods and kits of the invention is not a naturally occurring antibody. Specifically, any of the antibodies used herein cannot be considered as a product of nature. In yet some further embodiments, immobilization of any of the antibodies used to create an immobilized metabolite-fibril/s specific antibody, clearly distinguishes the product used from its natural counterpart. Still further, the antibodies used in the diagnostic methods and kits of the present disclosure specifically recognize and bind metabolite-fibril/s. Non-limiting embodiments of such antibodies are disclosed by the preset examples. As shown by FIGS. 12A and 12B, only the specific antibodies of the present disclosure that were prepared against the metabolite-fibril/s, specifically, the Hcy-fibril/s, and as opposed to the commercial antibodies used, recognize the specific Hcy-fibril/s also in brain sections of AD model mice. Thus, the antigen recognized by the antibodies is metabolite-fibril/s, specifically, the Hcy-fibril/s. Still further, in some embodiments, the Hcy-fibril/s were prepared as described in the examples. In yet some further embodiments, the metabolite-fibril/s used as an antigen were further characterized using several techniques and therefore display specific structural properties. For example, as shown in FIG. 1 and FIG. 2 (cryo-TEM micrographs), and FIG. 3 and FIG. 4 (IMS-MS analysis). These metabolite-fibril/s, and specifically, Hcy-fibril/s used as an antigen for antibody preparation, comprise at least one epitope that is specifically recognized by the antibodies, and is not recognized by antibodies directed against the metabolite. Such epitope is specific for the fibrillar form of the metabolite. The term “epitope” is meant to refer to that portion of any molecule or structure, e.g., metabolite-fibril/s, capable of being bound by an antibody which can also be recognized by that antibody. Epitopes or “antigenic determinants” usually consist of chemically active surface groupings of molecules such as amino acids or sugar side chains and have specific three dimensional structural characteristics as well as specific charge characteristics.

It should be appreciated that antibodies and antigens as specified herein, may be also applicable in any other aspect of the invention disclosed herein after.

The diagnostic methods and kits of the present disclosure are applicable for any sample obtained from the tested subject. As used herein, the term “sample” refers to cells, sub-cellular compartments thereof, tissue or organs. The tissue may be a whole tissue, or selected parts of a tissue. Tissue parts can be isolated by micro-dissection of a tissue, or by biopsy, or by enrichment of sub-cellular compartments. The term “sample” further refers to healthy as well as diseased or pathologically changed cells or tissues. Hence, the term further refers to a cell or a tissue associated with a disease, such a specific proteinopathy and/or protein misfolding disorder, for example, AD. A sample can be cells that are placed in or adapted to tissue culture. A sample can additionally be a cell or tissue from any mammalian species, specifically, humans. A tissue sample can be further a fractionated or preselected sample, if desired, preselected or fractionated to contain or be enriched for particular cell types.

In some specific and non-limiting embodiments, the sample of the method of the invention may be a body fluid sample. More specifically, such sample may be any body fluid such as plasma, cerebrospinal fluid (CSF), lymph, urine, serum and the like. The sample can be fractionated or preselected by a number of known fractionation or pre selection techniques. A sample can also be any extract of the above. Thus, in some specific embodiments, the sample may be any one of a biological sample of organ/s, cell/s or tissue/s and a blood sample.

It should be further appreciated that the diagnostic methods and kits further encompasses the option of combining antibodies that detect various metabolite fibrils, and/or of combining detection means for detecting the misfolded proteins, for example, methods and kits using antibodies that detect beta amyloid assemblies or aggregates, combined with antibodies that detect the Hcy-fibril formation.

A further aspect of the present disclosure relates to methods for treating, preventing, ameliorating, reducing or delaying the onset of at least one proteinopathy and/or protein misfolding disorder. More specifically, the method comprising the following steps. The first step (a), involves contacting at least one biological sample of the subject with at least one antibody specific for at least one metabolite-fibrils. The next step (b), involves classifying the subject as a subject affected by the at least one protein misfolding disorder or proteniopathy, if the metabolite-fibril is detected in the sample. The next step (c), involves administering to a subject classified as affected by the at least one protein misfolding disorder or proteniopathy, a therapeutically effective amount of at least one anti-proteinopathy or an anti-protein misfolding disorder therapeutic agent.

All scientific and technical terms used herein have meanings commonly used in the art unless otherwise specified. The definitions provided herein are to facilitate understanding of certain terms used frequently herein and are not meant to limit the scope of the present disclosure.

Before specific aspects and embodiments of the invention are described in detail, it is to be understood that this invention is not limited to particular methods, and experimental conditions described, as such methods and conditions may vary. It is also to be understood that the terminology used herein is for the purpose of describing particular embodiments only, and is not intended to be limiting, since the scope of the present invention will be limited only by the appended claims.

As used in this specification and the appended claims, the singular forms “a”, “an”, and “the” include plural references unless the context clearly dictates otherwise. Thus, for example, references to “a method” includes one or more methods, and/or steps of the type described herein and/or which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps. More specifically, the terms “comprises”, “comprising”, “includes”, “including”, “having” and their conjugates mean “including but not limited to”. This term encompasses the terms “consisting of” and “consisting essentially of”. The phrase “consisting essentially of” means that the composition or method may include additional ingredients and/or steps, but only if the additional ingredients and/or steps do not materially alter the basic and novel characteristics of the claimed composition or method.

The term “about” as used herein indicates values that may deviate up to 1%, more specifically 5%, more specifically 10%, more specifically 15%, and in some cases up to 20% higher or lower than the value referred to, the deviation range including integer values, and, if applicable, non-integer values as well, constituting a continuous range. As used herein the term “about” refers to f 10%.

It should be noted that various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible sub ranges as well as individual numerical values within that range. For example, description of a range such as from 1 to 6 should be considered to have specifically disclosed sub ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range. Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between.

The following examples are put forth so as to provide those of ordinary skill in the art with a complete disclosure and description of how to make and use the methods and compositions of the invention and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

The examples are representative of techniques employed by the inventors in carrying out aspects of the present invention. It should be appreciated that while these techniques are exemplary of preferred embodiments for the practice of the invention, those of skill in the art, in light of the present disclosure, will recognize that numerous modifications can be made without departing from the spirit and intended scope of the invention.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub combination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

Disclosed and described, it is to be understood that this invention is not limited to the particular examples, methods steps, and compositions disclosed herein as such methods steps and compositions may vary somewhat. It is also to be understood that the terminology used herein is used for the purpose of describing particular embodiments only and not intended to be limiting since the scope of the present invention will be limited only by the appended claims and equivalents thereof.

It must be noted that, as used in this specification and the appended claims, the singular forms “a”, “an” and “the” include plural referents unless the content clearly dictates otherwise.

### EXAMPLES

Materials

L-Homocysteine was purchased from CHEM-IMPEX with purity >98%. Other materials (methionine, EGCG, TA) were purchased from Sigma. Fresh stock solutions were prepared by dissolving the metabolites at 90° C. in PBS or Dulbecco's modified Eagle's medium (DMEM)/Nutrient Mixture F12 (Ham's 1:1, Biological Industries, without FBS) at various concentrations, followed by gradual cooling of the solutions.

Experimental Procedures

Transmission Electron Microscopy

Hcy was dissolved at 90° C. in PBS at various concentrations followed by gradual cooling of the solution. Where indicated, EGCG or TA (final concentration of 1 mM and 0.1 mM, respectively) were added before gradual cooling. Subsequently, 10 μL samples were placed on 400-mesh copper grids. After 2 min, excess fluids were removed. Samples were viewed using a JEOL 1200EX electron microscope operating at 80 kV.

Cryo-Transmission Electron Microscopy

Hcy solutions were prepared as described for TEM. Vitrified specimens were prepared on a copper grid coated with a perforated lacy carbon 300=mesh (Ted Pella Inc.). A 3 μL drop from the solution was applied to the grid and blotted with a filter paper to form a thin liquid film of solution. The blotted sample was immediately plunged into liquid ethane at its freezing point (−183° C.). The procedure was performed automatically in the Plunger (Lieca EM GP). The vitrified specimens were transferred into liquid nitrogen for storage. The samples were analyzed using a FEI Tecnai 12 G2 TEM, at 120 kV with a Gatan cryo-holder maintained at −180° C., and images were recorded on a slow scan cooled charge-coupled device CCD camera (Gatan). Images were recorded using the Digital Micrograph software package, at low dose conditions, to minimize electron beam radiation damage. Measurements were performed at the Ilse Katz Institute for Nanoscale Science and Technology (Ben-Gurion University of the Negev, Beer Sheva, Israel).

Powder X-Ray Diffraction (PXRD)

Lyophilized Hcy powder was dissolved to a concentration of 2 mg/mL in double distilled water and allowed to self-assemble by incubating at 18° C. for one week. The sample was then centrifuged for 10 min at 6000 rpm and the solution was decanted to remove non-assembled molecules. The assembled fibers were lyophilized and poured inside a glass capillary 0.7 mm in diameter. X-ray diffraction was collected using a Bruker D8 Discover diffractometer with LYNXEYE EX linear position detector. The capillary setup employed was as follows: Göbels mirrors to obtain a parallel beam, rotating capillary holder and 28 scan between 2 and 50°, step 0.02 Å. The presence of three NaCl peaks was noticed in the diffraction pattern. As it was not possible to obtain a sample without the presence of NaCl, these peaks were excluded in the further data treatments. Crystallographic structure determination was performed using the EXPO2014 software (77). EXPO2104 was used with cell indexing (N-TREOR09 algorithm) and the Simulated Annealing Method.

The solution with the lowest Cost Function was used as model to perform further crystal refinement on the structure using the GSASII software (78). The final error indexes were wR=5.83% and GoF=3.46. The crystallographic data have been deposited in the CCDC with no. 1976215.

ThT Kinetics Assay

Hcy was dissolved at 90° C. in PBS to a final concentration of 10, 5 and 2 mg/mL and maintained at 90° C. until the beginning of the measurement. Samples containing polyphenols were immediately mixed with the inhibitors. EGCG or TA (final concentration of 1 mM and 0.1 mM, respectively). As a control, Hcy was diluted with PBS alone to the same final concentrations. ThT in PBS was added to a final concentration of 20 μM for 5 and 10 mg/ml Hcy or 40 μM for 2 mg/ml Hcy. Next, the solutions were plated in a black 96-well, clear, and flat bottom microplate (Greiner) and self-assembly kinetics were recorded over time at room temperature (uncontrolled gradual cooling of the solutions) with short shaking before each read. ThT emission data at 480 nm (excitation at 440 nm), were measured using a Tecan™ SPARK 10 M plate reader, gain of 100 for 2 mg/ml, and using a Tecan™ Infinite 200 PRO plate reader, gain of 90 for 5 and 10 mg/ml. The displayed results are representative of three experiments performed in triplicates.

Auto-Fluorescence Kinetics Assay

Hcy was dissolved at 90° C. in PBS to a final concentration of 10 and 5 mg/mL and maintained at 90° C. until the beginning of the measurement. Next, the solutions were plated in a black 96-well, clear, and flat bottom microplate (Greiner) and self-assembly kinetics were recorded over time at room temperature (uncontrolled gradual cooling of the solutions) with short shaking before each read. Emission data at 450 nm (excitation at 375 nm) were measured using a Tecan™ SPARK 10 M plate reader. The displayed results are representative of three experiments performed in triplicates.

ThT Fluorescence Endpoint Measurements

Hcy was dissolved at various concentrations, ranging from 1 mg/mL to 12 mg/mL at 90° C. in PBS and plated on a 96-well black plate together with 20 μM ThT in PBS (final concentration). Following an overnight incubation at room temperature with gentle rotation (uncontrolled gradual cooling of solutions), ThT emission signal at 480 nm (excitation at 450 nm) was measured at room temperature with short shaking before each read and using a Tecan™ Infinite 200 PRO plate reader, gain 90. The displayed results are representative of three experiments performed in triplicates.

Cytotoxicity Experiments

SH-SY5Y cells (ATCC CRL-2266) or HEK293 cells (ATCC® CRL-1573) were cultured (2×10⁵ cells/mL) in DMEM/Nutrient Mixture F12 (Ham's; 1:1) supplemented with 10% fetal bovine serum (FBS) in 96-well tissue microplates (100 μL per well) and allowed to adhere overnight at 37° C. Half of each plate was plated with cells, with the other half later serving as a control containing solutions alone. The treatment solutions were prepared as follows: Hcy or methionine were dissolved at 90° C. and at various concentrations in cell media without FBS, followed by gradual cooling of the solutions. For solutions containing polyphenols, Hcy (final concentration of 2 mg/mL) was similarly dissolved in cell media and mixed with EGCG or TA (final concentration of 0.1 mM and 0.01 mM, respectively, stock solution dissolved in cell medium) before gradual cooling. The medium was replaced and cells were treated with the solutions (100 μL per well), followed by overnight incubation at 37° C. Control cells were incubated with medium that was treated in the same manner (without Hcy). Controls of media supplemented with the inhibitors, without Hcy, were examined as well. Cell viability was evaluated using the 3-(4,5-dimethylthiazolyl-2)-2,5-diphenyltetrazolium bromide (MTT) assay. Briefly, 10 μL of 5 mg/mL MTT dissolved in PBS was added to each well. After a 4 hours incubation at 37° C. 100 μL of extraction buffer 120% SDS dissolved in a mixture of 50% DMF and 50% DDW (pH 4.7)] was added to each well, and the plates were further incubated at 37° C. for 30 min. Finally, color intensity was measured using an ELISA reader at 570 nm. Blank measurements of the solutions without cells were respectively subtracted. The data points are presented as mean±SD. For inhibitors and methionine controls, two-tailed Student's t test was performed when two groups were compared. The displayed results are representative of three biological experiments performed in triplicates.

Apoptosis Assay

SH-SY5Y cells were cultured at 2×10⁵ cells/mL in 24-well plates in DMEM/Nutrient Mixture F12 (Ham's; 1:1) supplemented with 10% FBS and were allowed to adhere overnight at 37° C. The treatment solutions containing Hcy and methionine were prepared as described above for the MTT assay. Cells were treated with the solutions followed by overnight incubation at 37° C. Control cells were incubated with medium that was treated in the same manner without Hcy. The apoptotic effect was evaluated using the MEBCYTO Apoptosis kit (MBL International), according to the manufacturer's instructions. Briefly, the adherent cells were trypsinized, detached and combined with floating cells from the incubated growth medium. Cells were then centrifuged and washed once with PBS and once with binding buffer. Cells were subsequently incubated with annexin V-FITC and PI for 15 min in the dark, resuspended in 200 μL binding buffer and analyzed by flow cytometry using a single laser-emitting excitation light at 488 nm. Data from at least 10⁴ cells were acquired using the BD FACSort and the CellQuest software (BD Biosciences). Analysis was performed using the FlowJo software (TreeStar, version 14). The displayed results are representative of three biological experiments performed in triplicates.

Construction of cys4Δ Yeast Strain

The cys4 gene was disrupted in wild-type (WT) yeast strain (BY4741) via homologous recombination using PCR fragments amplified from the plasmid pFA6a-KanMX6 as a template with primer DL154:

(5′GATTTCGTTGTAGGCCACTTGCTCAAAGGACATCT
AGATAAATACGACGTAAGAATAAAACGGATCCCCGGGT
TAATTAA-3′, as denoted by SEQ ID NO. 1)
and
primer DL155:
(5′AGGAAAGGAATGACGGATTTTGCTTCTATGTTTCTTT
TATTTGAAGCGTGGGTTCTTATACATTTGATTAAAATAG
AAC-3′, as denoted by SEQ ID NO. 2)

where bold underlining indicates the plasmid sequence.

The PCR fragments contain homology regions in each side and a with a selectable marker gene in the middle. The fragments are integrated into yeast chromosomes during yeast transformation by homologous recombination.

Gene replacement was validated by PCR with suitable primers that confirm the presence of the desired fragment in the right place.

Yeast Growth Assays

WT and cys4Δ strains were grown overnight at 30° C. Unless otherwise specified, the strains were grown on synthetic defined medium (SD) containing 20 mg/L L-methionine, 20 mg/L L-histidine monohydrochloride monohydrate, 20 mg/L uracil and 30 mg/L L-leucine. Cysteine was added to a final concentration of 13 mg/L. Hcy was added at the indicated concentrations.

For spotting assays, strains were diluted to 6.25*10⁷ cells/mL and then 5-fold serially diluted and spotted on the indicated plates. Plates were incubated at 30° C. for 2-3 days. The displayed results are representative of three biological experiments.

For OD₆₀₀ measurements, strains were diluted to OD₆₀₀ 0.01. 200 μL of cells were platted on 96 wells plates and incubated at 30° C. for 30 hours with continuous shaking. OD₆₀₀ was measured using Tecan™ SPARK 10M plate reader. The displayed results are representative of three biological experiments performed in triplicates.

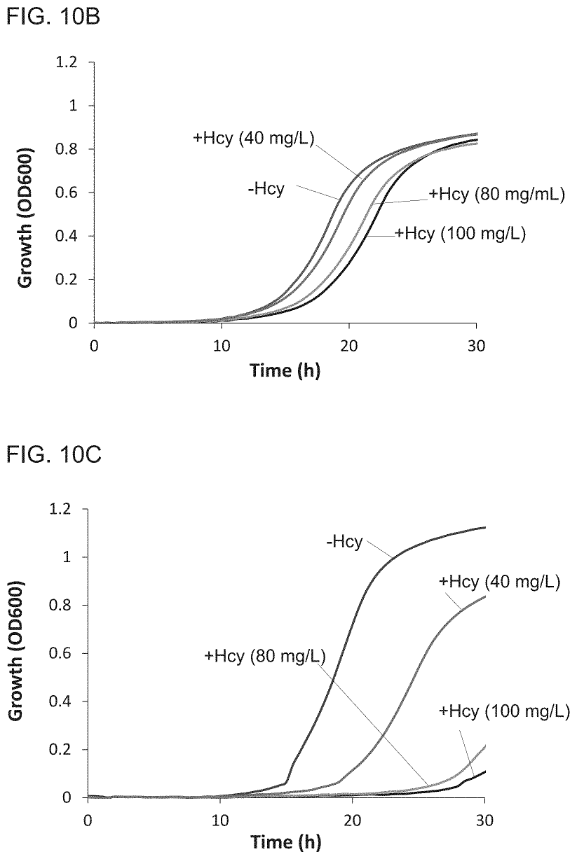

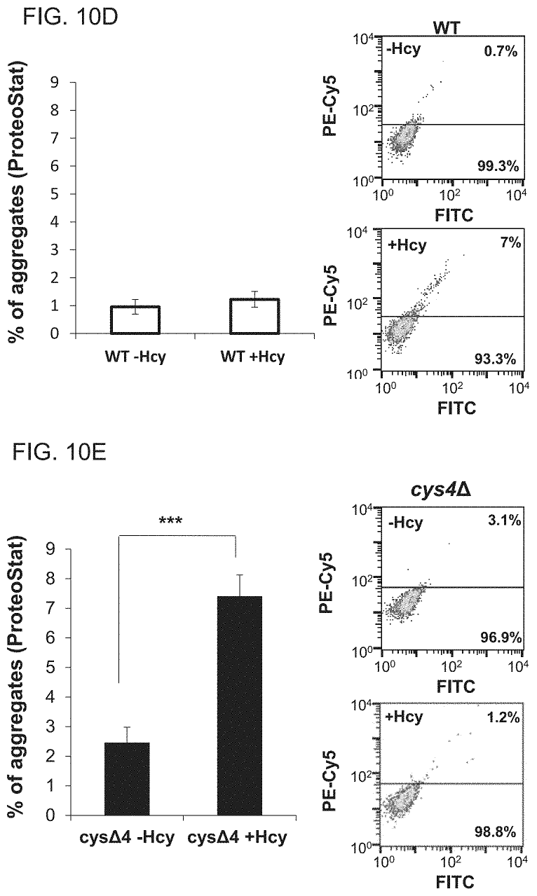

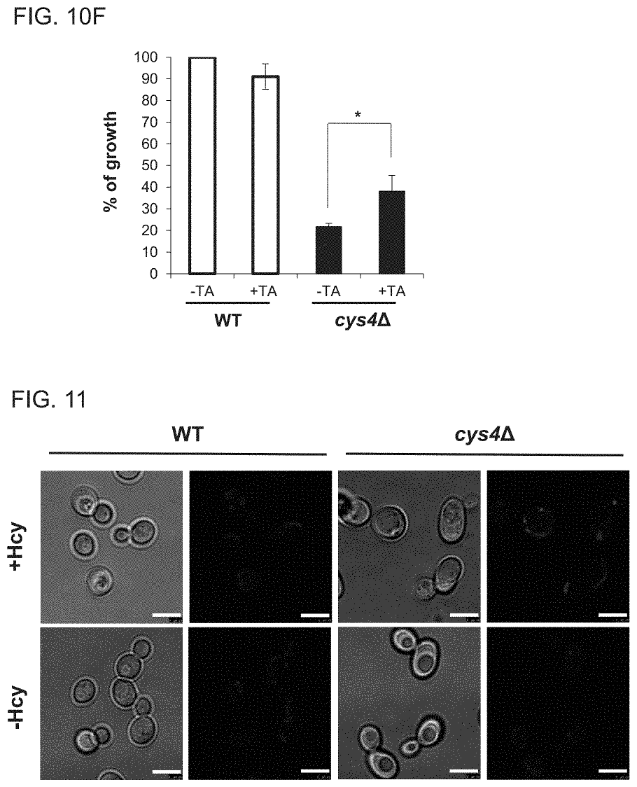

The percentage of growth analysis (FIG. 10F) represents the OD₆₀₀ levels following 27 hours of growth in the plate reader. The percentage of growth represents the growth under the indicated conditions, with or without 0.3 mM TA, compared to the growth of WT without TA. The displayed results are representative of three biological experiments performed in triplicates.

Aggregation Level Assays in Yeast

For each sample, 2×10⁶ cells/mL of logarithmic cells were resuspended with ProteoStat dye (Enzo Life Sciences). Cells were incubated for 15 min at room temperature protected from light. Flow cytometry was preformed using Stratedigm S1000EXi and the CellCapTure software (Stratedigm, San Jose, Calif.). Live cells were gated (P1) by forward scatter and side scatter. Fluorescence channels for FITC (530/30) and PE-Cy5 (676/29) were used utilizing a 488 nm laser source. A total of 50,000 events were acquired for each sample. Analyses were performed using the FlowJo software (TreeStar, version 14). The displayed results are representative of three biological experiments performed in triplicates.

Mouse Model of AD

5×FAD model mice co-overexpress a familial AD (FAD) mutant form of human amyloid precursor protein (APP) and human Presenilin1 (PS1). These double transgenic mice co-express five FAD mutations (5×FAD mice: the Swedish mutation, K670N/M671L; the Florida mutation, I716V; the London mutation, V717I, and PS1 M146L/L286V trans-genes) which increase β-amyloid production. β-amyloid deposition is first detected in the brain of the model mice in the subiculum of the hippocampal area. Mice develop β-amyloid plaques (and gliosis) by 5-6 weeks of age, show robust inflammation and neuronal damage and reach a very large plaque burden, especially in the subiculum and deep cortical layers (65, 66). Studies were performed using three 13-month-old age 5×FAD female mice and 3 age-matched WT female mice.

All mice were euthanized using CO₂ and intracardially perfused with Ringer's solution (NaCl 6.5 gL⁻¹, KCl 0.42 gL⁻¹, CaCl₂) 0.25 gL⁻¹, NaHCO₃ gL⁻¹, containing 4 units mL⁻¹ of Heparin in DW) followed by 4% paraformaldehyde (PFA) in PBS. Brains were collected and post-fixed with 4% PFA overnight at 4° C. before treated by a sucrose gradient: 15% sucrose in PBS overnight followed by 30% sucrose in PBS overnight. Brains were preserved in Peel-A-Way disposable plastic tissue-embedding-molds (Sigma #E6032) filled with tissue freezing media (OCT) and stored at −80° C. until sectioning. Coronal tissue sections (20 μm thick) were obtained using Leica CM 1950 Cryostat. Sections were placed on glass slides, air-dried, and kept at −20° C. until staining. All experiments were in accordance with Tel Aviv University guidelines and approved by the Tel Aviv University Institutional Animal Care and Use Committee (Protocol Number: 04-17-033).

Immunohistological Staining of Mouse Brain Tissue

Samples were washed three times in PBS (3×5 min), fixed with 4% PFA (in PBS) for 10 min, washed (3×5 min) in PBS, and then permeabilized for 10 min in 0.5% Triton-X 100 (Sigma-Aldrich) in PBS. The slices were blocked with a blocking solution containing 0.5% Triton, 0.3 M glycine, 1% BSA solution (in PBS) for 1 h, then incubated with primary rabbit anti-Hcy-fibrils antibodies overnight at 4° C. (purified by Adar Biotech; 1:200 in blocking solution) or similarly with commercial antibodies from Abcam (#ab15154) and Sigma-Aldrich (#AB5512). On the next day, the samples were washed three times in PBS and incubated with Alexa F488-conjugated goat-anti-rabbit secondary antibody diluted 1:500 in PBS for 1 h at 25° C. (ThermoFisher; #A11008).

For double immunofluorescence staining, the same sections were washed with PBS (3×5 min), blocked again with a blocking solution containing 8% (v/v) horse serum, 0.3% (v/v) Triton, 1% (w/v) BSA, 0.02% (v/v) sodium azide solution (in PBS) for 1 h, and then incubated with primary mouse anti-AD antibodies (6E10. BioLegend: #803001) or primary rat anti-GFAP antibodies (GFAP, Millipore; #345860) diluted 1:500 in blocking solution, overnight at 4° C. After washing (PBS 3×5 min), the sections were incubated with Alexa F594-conjugated goat anti-mouse (ThermoFisher; #A11005) or goat anti-rat (ThermoFisher: #A11007) secondary antibody diluted 1:500 in PBS, for 1 h at 25° C., and co-stained with DAPI diluted 1:500 in PBS for 10 min to detect nuclei (DAPI, Sigma-Aldrich). Sections were then mounted in Vectashield mounting medium (Vector Labs) and coverslipped. Immunofluorescence-stained brain sections were observed and images were acquired at ×10 magnification using Nikon Eclipse 80i fluorescence microscope (Melville, N.Y., USA) and MicroPublisher 6 camera (Teledyne QImaging). The displayed results are representative of three independent biological repeats.

Ion Mobility Spectrometry-Mass Spectrometry Analysis

A 10 mM Hcy stock solution was prepared by dissolving 2.6 mg Hcy in 2 mL LC-MS grade water (OmniSolv). After a week, the stock was further diluted 1:10 to a final concentration of 0.13 mg/mL (0.96 mM) in 20 mM ammonium acetate pH 7 (VWR, Life Science). The mass spectrometry and multi-field ion-mobility spectrometry experiments were performed on an Agilent 6560 IMS-Q-TOF instrument (Agilent Technologies Santa Clara, Calif.). The sample was injected into a dual ESI/Agilent Jet Stream using a syringe pump at a rate of 2.5 μL/min. The drying gas was set at 5 L/min at a temperature of 300° C. The sheath gas was set at 11 L/min, 25 psi and 250° C. The ions were accumulated and stored in the entrance funnel for 5 ms, and a short pulse of 150 μs was subsequently used to inject the ions into the 78.1-cm drift cell filled with N₂ at 3.94 torr. Five different drift voltages were chosen in the multi-field experiment (ΔV=1450, 1350, 1250, 1150 and 1050). A transmission tune in fragile ion mode and collisional cross-section (CCS) measurements using Agilent tune-mix (Santa Clara, Calif.) were performed prior to data acquisition. The data were visualized using the IM-MS Browser 10.0 and the drift time data were used to calculate the experimental CCSs using the Mason-Schamp equation [Mason, E. A. & McDaniel, E. W, NASA STI/Recon Technical Report a, 89, 15174 (1988)].

Theoretical CCSs were calculated using the trajectory method available in the Mobcal package (with helium as the buffer gas [Mesleh. M. F., et. al., J. Phys. Chem. 100, 16082-16086 (1996); Shvartsburg, A. A. & Jarrold. M. F. et. al., Chem. Phys. Lett. 261, 86-91 (1996)]. To convert the CCS_{He} to CCS_{N2}, the CCSs of the Agilent tune-mix ions were used: m/z 322, 622, 922, 1222, and 1522 in He and N₂ for calibration. The experimental CCSs (in squared angstrom) of these ions in He are 98.2, 138.2, 175.2, 209.2, and 239.4, respectively. In N₂, the corresponding values are 153.4, 202.4, 242.7, 280.9, and 315.7 [Kurulugama, R. T., et. al., (2015) Analyst 140, 6834-6844]. Using these values, the conversion equation of CCS_{N}_{₂}=1.1412×CCS_{He}+42.713 was obtained.

Confocal Microscopy

SH-SY5Y cells: 2×10⁵ cells/mL were cultured in DMEM/Nutrient Mixture F12 (Ham's; 1:1) supplemented with 10% FBS in 24-well plate on glass slides coated with 0.01% Poly-L-Lysine. When reaching ˜70-80% confluency, cells were treated with Hcy (2 mg/mL dissolved at 90° C. in cell media without FBS, followed by gradual cooling of the solution). After 4 hours of incubation, the cells were immediately supplemented with ProtesoStat dye diluted 1:250 in ProteoStat assay buffer followed by incubation for 15 min at room temperature protected from light. Cells were imaged using Leica TCS SP8 laser confocal microscope with ×63 1.4 NA or ×100 1.4 NA oil objectives. An argon laser with 488 excitation line was used for ProteoStat (emission wavelength, 500-600 nm). The results displayed are representative of three biological experiments.

Yeast cells: 1 ml of logarithmic cells were washed with PBS buffer and sonicated using 15 s pulses at 20% power. Cells were resuspended in 50 μL of ProtesoStat dye diluted 1:250 in ProteoStat assay buffer followed by incubation for 15 min at room temperature protected from light. 10 μL of each sample was deposited on poly-lysine-coated glass slides (Sigma-Aldrich). Cells were imaged using Leica TCS SP8 laser confocal microscope with ×63 1.4 NA or ×100 1.4 NA oil objectives. An argon laser with 488 excitation line was used (500-600 nm emission). The results displayed are representative of three biological experiments.

Generation of Anti-Hcy-Fibrils Antibodies

Hcy assemblies, formed as outlined, served as antigens in a series of immunization cycles in rabbits (ADAR BIOTECH, Rehovot, Israel). ELISA was employed to validate the specificity of the antibody to Hcy fibrils. To purify antibodies towards anti-metabolite assemblies, the Hcy assemblies served as antigens in a series of immunization cycles in rabbits.

To create Hcy assemblies, 2 mg/ml Hcy was dissolved at 90° C. in PBS followed by gradual cooling of the solution.

The Hcy assemblies were injected into two rabbits, followed by seven immunization cycles, with intervals of 3 weeks between each immunogen. The rabbit's serums were collected 7-10 days after the last injections and combined. Approximately 40 ml from each Rabbit (total of 80 ml sera). Antibodies were purified from the rabbit serum, via protein A column separation, specifically, a Protein A affinity column lot #1003 66 (3 ml bed volume) according to GE purification protocol. Purified polyclonal IgG antibodies were collected and stored at −80° C. The polyclonal antibodies were used as a primary antibody in a reactivity assay. FIG. 12A-12B demonstrate the specificity of these antibodies to Hcy fibril structures, as opposed to two commercial antibodies that display no recognition of the fibril form of the metabolite.

Amine Binding ELISA

A fresh solution of Hcy assemblies (10 mg/mL) was used as antigen for the assay. Hcy assemblies (200 μL) were loaded in triplicates onto an “Amino Immobilizer” 96 well plate (Nunc) and allowed to adhere with gentle agitation at room temperature for 2 hours, and then incubated overnight at 4° C. PBS was also loaded as a control to rule out solvent background staining. On the following day, the plate was blocked with 3% BSA (VWR) in solution. Three different primary antibodies against Hcy were tested, two commercial antibodies from Abcam (#ab15154) and Sigma-Aldrich (#AB5512) and the rabbit anti-Hcy-fibrils antibodies specifically generated against Hcy assemblies in this study (diluted 1:500). Secondary antibody was diluted 1:5000. Blocking and primary antibody steps were performed for 2 hours each, while the secondary antibody was incubated for 1 hour. Between the stages, three wash cycles with TBS-T 0.1% were performed. 100 μL of 1-Step Ultra TMB ELISA Substrate (Thermo Scientific Pierce) were added to each of the wells followed by a 30 min incubation (until the appearance of blue color). The plate was then transferred to a CLARIOstar plate reader (BMG LABTECH) and the absorbance was measured at 605 nm. The displayed results are representative of three experiments performed in triplicates.

In-Vitro Seeding of β-Amyloid₁₋₄₂ by Hcy Assemblies

Hcy was dissolved at 90° C. in PBS followed by gradual cooling of the solution. D-amyloid₁₋₄₂ (rPeptide) was dissolved in DMSO to a concentration of 120 μM. The monomeric protein was mixed with pre-formed 20% v/v Hcy assemblies (taken from 10, 5 or 2 mg/mL stock solutions), or with an equivalent volume of PBS as a control, to a final concentration of 3 μM β-amyloid₁₋₄₂ ThT was added to a final concentration of 20 μM. Aggregation was monitored using Tecan™ Infinite 200 PRO fluorescence plate reader over time at 37° C. Blank measurements of Hcy only (without β-amyloid) were respectively subtracted. The displayed results are representative of three experiments performed in triplicates.

### Example 1

Self-Assembly of Hcy to Form Amyloid-Like Fibrils and their Structural Analysis

The inventors first examined whether Hcy, a sulfur-containing non-coded amino acid (FIG. 1A), can self-assemble into ordered supramolecular structures. To examine the possible in-vitro self-association of Hcy and to further characterize the assemblies, the inventors dissolved Hcy to obtain a homogenous solution, which resulted in its self-assembly into ordered fibrillary structures. As assessed by transmission electron microscopy (TEM), Hcy formed amyloid-like fibrils that could be detected by structure adsorption to a copper grid (FIG. 1B), as well as in the solution, as demonstrated using cryo TEM (FIG. 1C). Hcy fibrils could be detected at various concentrations (FIG. 2A-2B).

In spite of its vast biological importance, the crystal structure of Hcy has not been reported in the literature. To determine the molecular arrangement of Hcy at the atomic level and the overall packing, the inventors set out to solve the single crystal structure of Hcy. Due to the challenge of growing single crystals of sufficient size and quality for single-crystal diffraction, powder X-ray diffraction (PXRD) methods were employed to determine the crystal structure of Hcy, as previously used for the crystal structure determination of L-Lysine (51). The freeze-dried samples of self-assembled Hcy typically give rise to a microcrystalline powder of the anhydrous phase, thus requiring PXRD for structure determination. The solved structure from the PXRD pattern contained three molecules of Hcy in the unit cell with triclinic P-1 space group comprising a=10.27 Å, b=7.01 Å, c=14.15 Å, α=99.08°, β=75.75°, γ=68.31° (FIG. 1D). The crystal structure of Hcy can be described as a layered arrangement (a supramolecular β-sheet), common with the crystal structures of various single amino acids (52, 53). In the crystallographic b-direction, Hcy molecules were connected through H-bonds between head group NH₃⁺ and COO⁻ as well as side-chain —SH group, thereby producing a single sheet (FIG. 1E-i). FIG. 1f presents the top view of the H-bonded sheet. Two nearby sheets were connected to each other by further H-bonds and thus formed a single layer which can be signified as a single β-strand (FIG. 1E-ii) (54). The monolayers packed up to form a double layer assembly, in which side-chain to side-chain van der Waals forces connected the hydrophobic edges of the planes (FIG. 1E-iii). A hydrophobic region was in fact composed of a bilayer, while a hydrophilic region or a single layer was composed of two hydrophilic sheets. The continuous hydrophobic stacking of single layers or supramolecular β-strands fabricated the overall packing of Hcy, which resembled supramolecular β-sheet arrangement. The H-bonding directions inside the two sheets forming a layer was the same as those in next layer. Based on H-bonding direction, the higher order packing of Hcy can be classified as a parallel β-sheet structure. The organization into supramolecular β-sheet structure at the atomic level further supports the amyloidal characteristics of Hcy.

### Example 2

Ion Mobility Spectrometry-Mass Spectrometry (IMS-MS) Analysis of Hcy Self-Assembly

Similar to the previously reported case of phenylalanine (55), the mass spectrum of Hcy of FIG. 3, shows evidence of oligomerization under the gentle IMS-MS conditions. The IMS-MS experiments were performed in positive polarity with the fragile ion tune. The most abundant peak appeared at m/z 269, indicating a singly protonated dimer (n/z=2/1), while the protonated monomer was detected at m/z 136 (n/z=1/1; ˜30% of the dimer) (FIG. 3A). Large oligomers were assigned according to the observed mass to charge ratios (m/z) and isotope spacings. For example, in the range from m/z 1341 to 1345 (FIG. 3b-i), which corresponds to a nominal oligomer to charge (n/z) of 10/1, three isotope patterns of 1.0, 0.5 and 0.33 were observed. These unique spacings suggest that there are at least three distinct populations of oligomers: singly charged decamers (n=10), doubly charged eicosamers (n=20), and triply charged triacontamers (n=30). Following this approach, the largest oligomers identified in the mass spectrum were n=36. The singly protonated oligomers were observed from n=1 to n=12. Doubly protonated oligomers started at n=12 and at least up to n=24. Triply protonated oligomers were detected at n=32. Importantly, larger oligomers (n>36) were not observed due to the limited mass range of fragile ion mode (m/z 100-1700). Of note, only even-sized oligomers were observed in 24-h incubated samples, supporting that oligomer formation was not due to in-source clustering reactions. The exact masses of the oligomers suggest the later formation of disulfide bonds between every two Hcy monomers to stabilize the structure as the Hcy molecules are close in space (FIG. 3A, with panels (i) to (v), showing isotope distribution of the indicated oligomers). In addition, some high-mass spectral peaks showed two features (conformations) in their arrival time distributions (ATDs) with the same isotope spacing, implying distinct growth paths. For example, m/z 939 with an n/z=14/2 showed two prominent conformations (FIG. 3B-ii). Other mass spectral peaks with similar ATDs were m/z 1073 (n/z=16/2), 1209 (n/z=18/2), and 1475 (n/z=22/2).

The experimental collisional cross-sections (CCSs) were compared to different growth models, four of which were obtained from the PXRD data (FIG. 4A). The models include (a) the isotropic model, commonly used in peptide assembly assuming the monomer unit and oligomers are all globular (56), (b) the monolayer formed of two Hcy sheets stabilized via inter-sheet hydrophilic interactions, (c) the bilayer, similar to the monolayer but made of four sheets, (d) the single tube model where each side of the “tube” is made of three sheets, and (e) the double tube model (FIG. 4B). The isotropic model significantly deviates from the experimental data, as the monomeric Hcy is not globular. Deviations from this model were reported for other amino acids such as serine (57). The mono- and bilayer models also overestimate the CCSs. On the other hand, the single and double-tube models better account for the growth of Hcy oligomers, as indicated by the agreement between the predicted and experimental CCSs, suggesting them to be the two growth paths for Hcy assembly. The tube models provide sufficient hydrophobic and hydrophilic stability for the growth into fibrils. Overall, the IMS-MS data disclosed herein provide compelling evidence that Hcy oligomerizes into fibrils via the tube modes, resembling the case of phenylalanine (55).

### Example 3

Kinetic Analysis of Hcy Self-Assembly

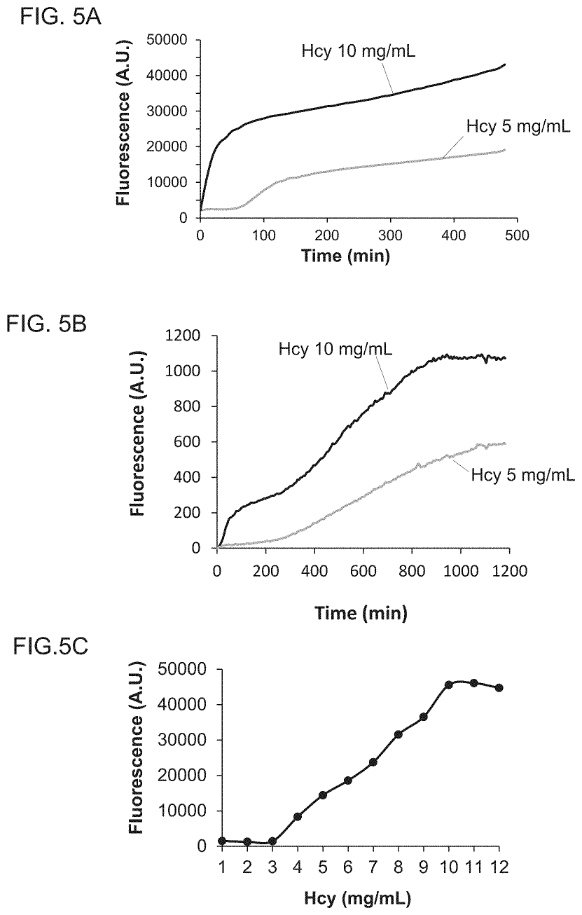

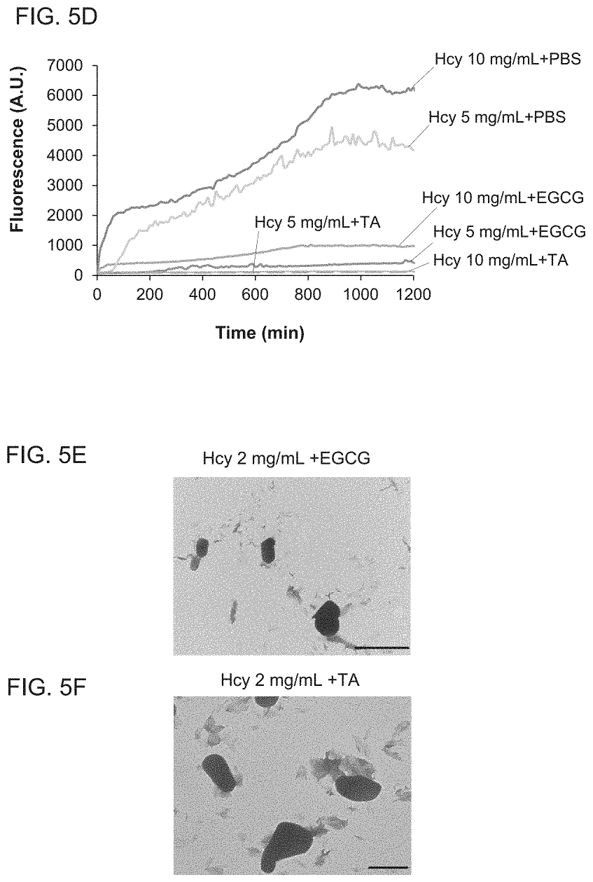

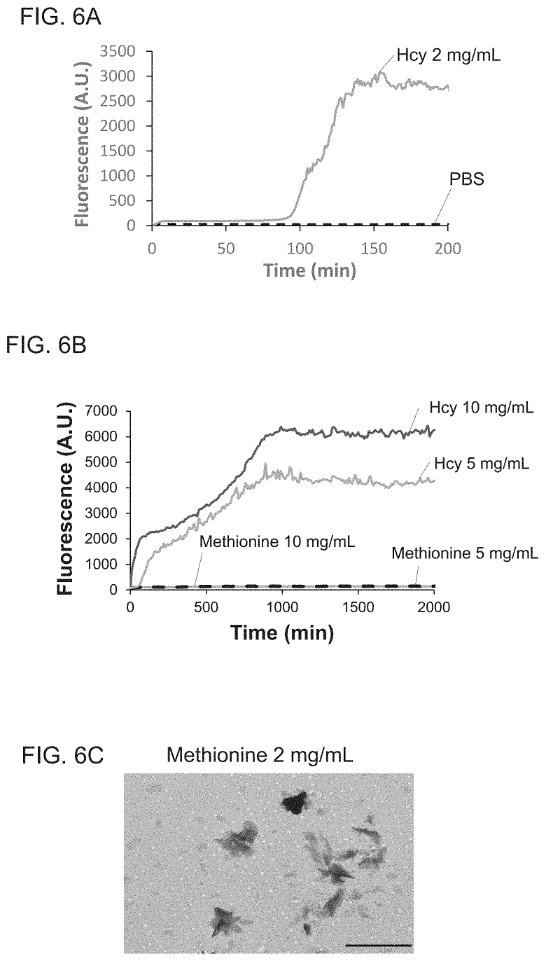

Metabolite fibrillar assemblies were previously reported to exhibit amyloid-like attributes, such as thioflavin T (ThT) binding, auto-fluorescence properties and a supramolecular β-sheet-like conformation (43, 58). Here, using ThT binding assays, the inventors could detect the kinetics of Hcy self-assembly, with higher metabolite concentrations showing stronger ThT fluorescence (FIG. 5A, FIG. 6A). The assembly kinetics could also be detected by the auto-fluorescence properties of Hcy (FIG. 53), as similarly demonstrated for other metabolite assemblies (58). A ThT binding assay similarly showed a sigmoidal self-assembly of Hcy at different concentrations (FIG. 5C) consistent with the mechanism of nucleation-growth typically observed for amyloid formation (59).

### Example 4

Inhibition of Hcy Fibril Formation by Polyphenols

Previous studies demonstrated that self-assembly of metabolite amyloids can be inhibited by polyphenols (48, 50). The inventors used epigallocatechin gallate (EGCG) and tannic acid (TA), two aggregation inhibitors recently reported to inhibit fibril formation by metabolites (48, 50). The inventors first examined the inhibitory potential of the polyphenols on Hcy self-assembly kinetics using ThT binding assays. The inhibitors were added at timepoint zero followed by monitoring of ThT fluorescence intensity (FIG. 5D). Both EGCG and TA were found to inhibit the formation of Hcy fibrils as reflected by the significant reduction in the ThT fluorescence intensity curves. Using TEM analysis, the inventors confirmed that no structures were formed in the mixture of Hcy with EGCG or TA (FIG. 5E, 5F) along with a control solution of Hcy alone that exhibited similar fibrils as presented in FIG. 1.

### Example 5

Cytotoxicity of Hcy Assemblies

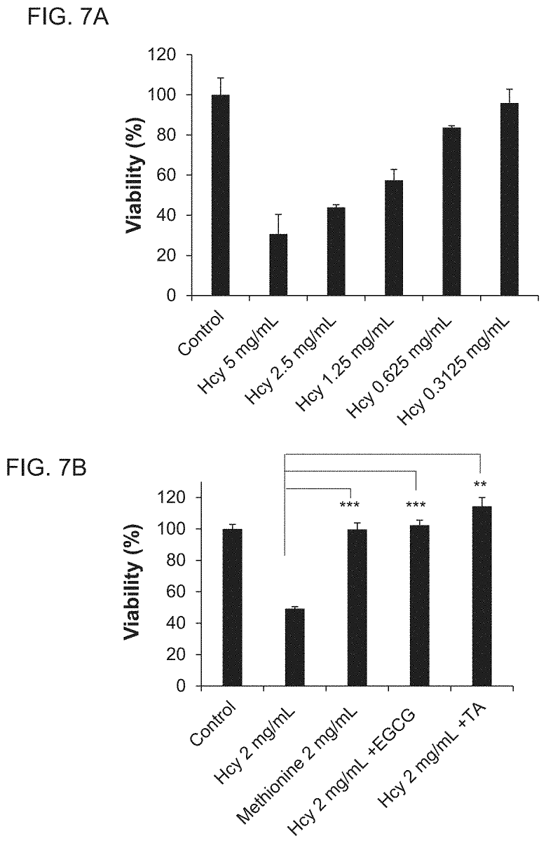

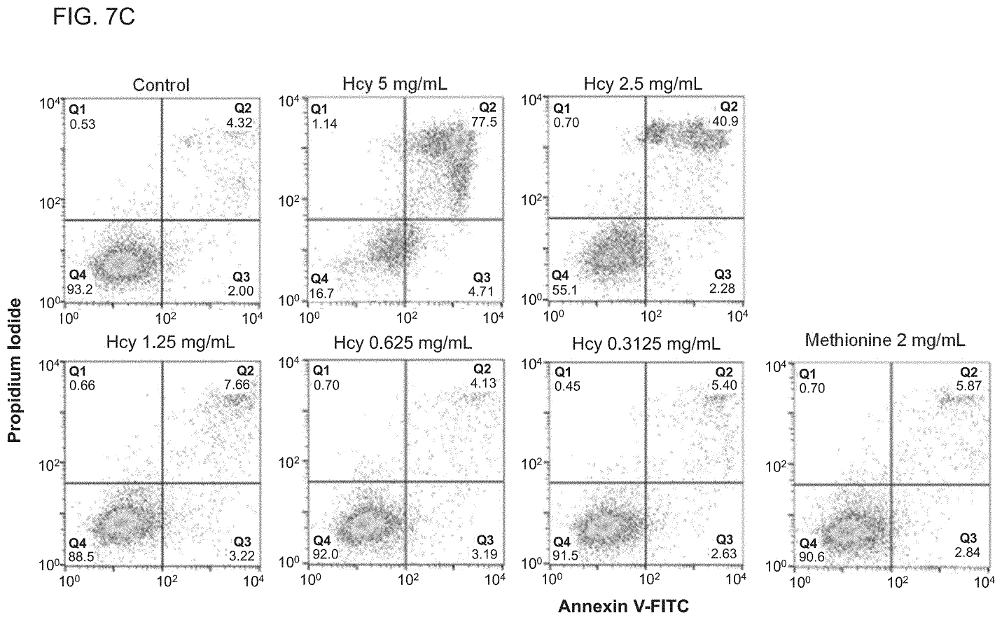

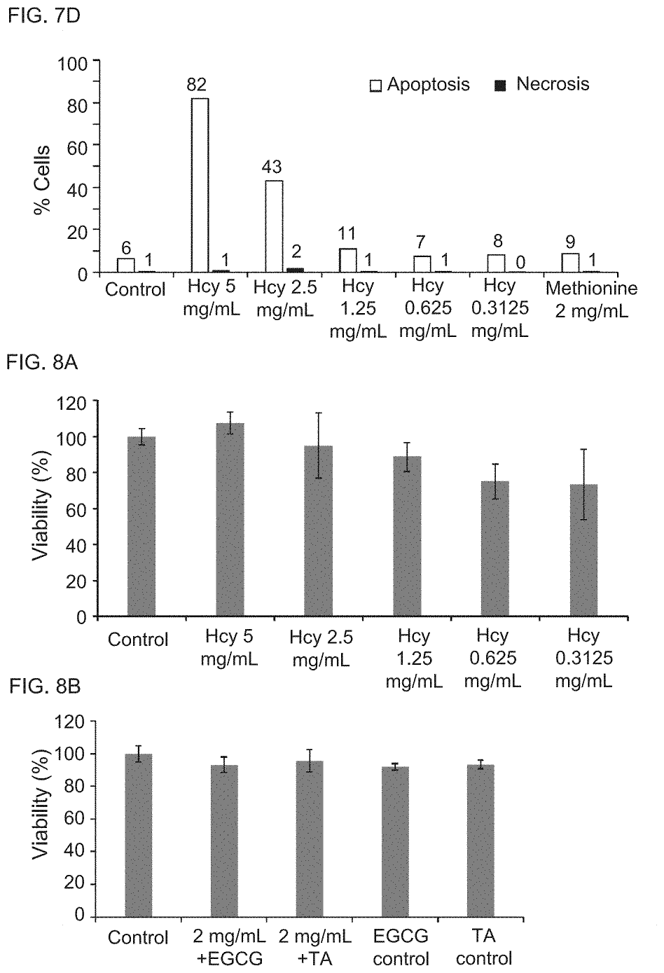

Previous studies demonstrated that metabolite amyloids bear a cytotoxic effect, inducing apoptotic cell death in cultured neuronal cells, similar to the effect of protein and polypeptide amyloids (43-45, 60, 61). The inventors therefore examined the cytotoxicity and apoptotic activity of Hcy assemblies on cultured human neuroblastoma cells (SH-SY5Y) (FIG. 7). The assemblies were prepared by dissolving a wide range of Hcy concentrations in culture media. Using MTT cell viability assay, the inventors tested the toxicity of the assemblies following a 24-hour treatment (FIG. 7). Medium without Hcy that was prepared in the same manner was used as a control. As a blank, measurements of Hcy assemblies dissolved in medium at the corresponding concentration (without cells) were subtracted from the cell viability data. Hcy assemblies displayed a dose-dependent cytotoxicity and caused up to 80% cell death (FIG. 7A). In addition, as Hcy is part of the S-adenosyl-methionine metabolic pathway (20), methionine was examined as a negative control. The inventors similarly prepared and treated cells with methionine solution in cell medium. The methionine solution did not show any toxicity towards the neuroblastoma cells, compared to Hcy solution at the same concentration (FIG. 7B). Using TEM analysis, the inventors confirmed that methionine did not form amyloid-like fibrils (FIG. 6C). In addition, methionine did not show a fluorescence signal when evaluated using a ThT assay and compared to Hcy (FIG. 6B). Notably, no significant toxicity was observed towards a non-neuronal HEK293 cell line (FIG. 8A), suggesting cell-type-dependent toxicity.

The inventors further examined the cytotoxicity towards SH-SY5Y cells following inhibition of Hcy self-assembly by EGCG and TA. The inhibitors were added to the Hcy solutions prepared in cell media before gradual cooling. As described above, SH-SY5Y cells were treated with cell media containing Hcy that was supplemented with EGCG or TA, for 24 hours (FIG. 7B). When treating cells with inhibitors-containing solutions, the MTT assay indicated a significant increase in cell viability compared to Hcy solutions in the absence of EGCG or TA (FIG. 7B). The inventors further verified that the inhibitors alone did not affect cell viability (FIG. 8B). These results validate the amyloid-like properties of Hcy fibrils and confirm Hcy structures, rather than an osmotic effect, as the toxicity-causing agent.

Next, the inventors examined whether the cytotoxicity of Hcy fibrils was due to apoptotic cell death. The inventors used annexin V and propidium iodide (PI) labeling, followed by flow cytometric analysis (FIG. 7C, 7D). Different concentrations of Hcy assemblies as well as methionine solution in cell media were prepared as described above, and cultured SH-SY5Y cells were treated for 24 hours. The Hcy assemblies caused cell death and stimulated apoptotic activity in a concentration-dependent manner, while the methionine negative control did not show any apoptotic effect, as indicated by annexin V and PI assay (FIG. 7C, 7D). These results demonstrate that apoptosis, rather than necrosis, was the main triggered pathway causing SH-SY5Y cell death following treatment with Hcy assemblies.

### Example 6

Detection of Amyloid-Like Structure of Hcy in SH-SY5Y Cells

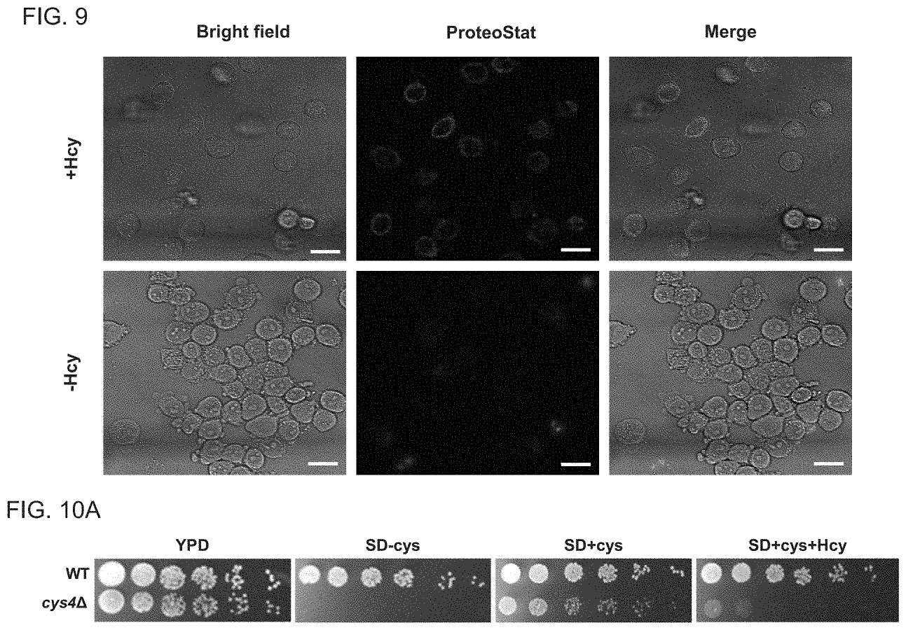

Following the observed cytotoxicity, the inventors sought to detect Hcy amyloid-like fibrils in treated SH-SY5Y cells. The cells were stained with the ProteoStat amyloid-specific fluorescent dye which was recently shown to be indicative of intracellular amyloid fibrils in yeast (48). The inventors similarly prepared solutions of Hcy assemblies (2 mg/mL) by dissolving the metabolite in cell culture medium followed by the treatment of SH-SY5Y cells for 4 hours. Medium without Hcy that was prepared in the same manner was used as a control. The treated and untreated cells were observed using confocal microscopy (FIG. 9), demonstrating the amyloid-like structures in treated SH-SY5Y cells, when the untreated cells did not show any detection.

### Example 7

In-Vivo Yeast Model for Hcy Toxicity

Next, the inventors sought to establish an in-vivo model for the study of Hcy accumulation and toxicity. The budding yeast Saccharomyces cerevisiae has been successfully used as a model organism for the study of metabolite aggregation, as well as of amyloid-associated diseases (48, 49). The inventors therefore aimed to establish a S. cerevisiae model of Hcy accumulation. Based on the etiology of hyperhomocysteinemia (28, 29), a knockout mutation in CYS4, the highly-conserved functional yeast CBS ortholog, was constructed. Due to the contribution of CYS4 to cysteine biosynthesis (62), its absence conferred cysteine dependence (FIG. 10A). Indeed, sensitivity to Hcy supplied in the growth medium was observed in the Hcy salvage model, compared to the wild-type (WT) strain, in a dose dependent manner (FIG. 10B, 10C). To examine whether the observed toxicity was associated with the formation of amyloid-like assemblies in-vivo, cells were stained with the amyloid-specific fluorescent dye ProteoStat. Upon Hcy feeding, a significantly higher degree of aggregation was detected by flow cytometry in the mutant compared to WT cells, as well as compared to the mutant strain in the absence of Hcy, indicating the presence of amyloid-like structures in the Hcy salvage mutant (FIG. 10D, 10E). Confocal microscopy imaging of the stained cells provided additional observation of the formation of amyloid-like assemblies in-vivo (FIG. 11). Next, the yeast model was used to validate the rescue of cytotoxicity by the polyphenol TA, as observed in cultured human neuroblastoma cells. While no effect was observed in the WT strain, the addition of TA significantly improved the cell growth of the mutant strain (FIG. 10F).

### Example 8

Hcy and β-Amyloid Interplay in Brain Sections of AD Model Mice

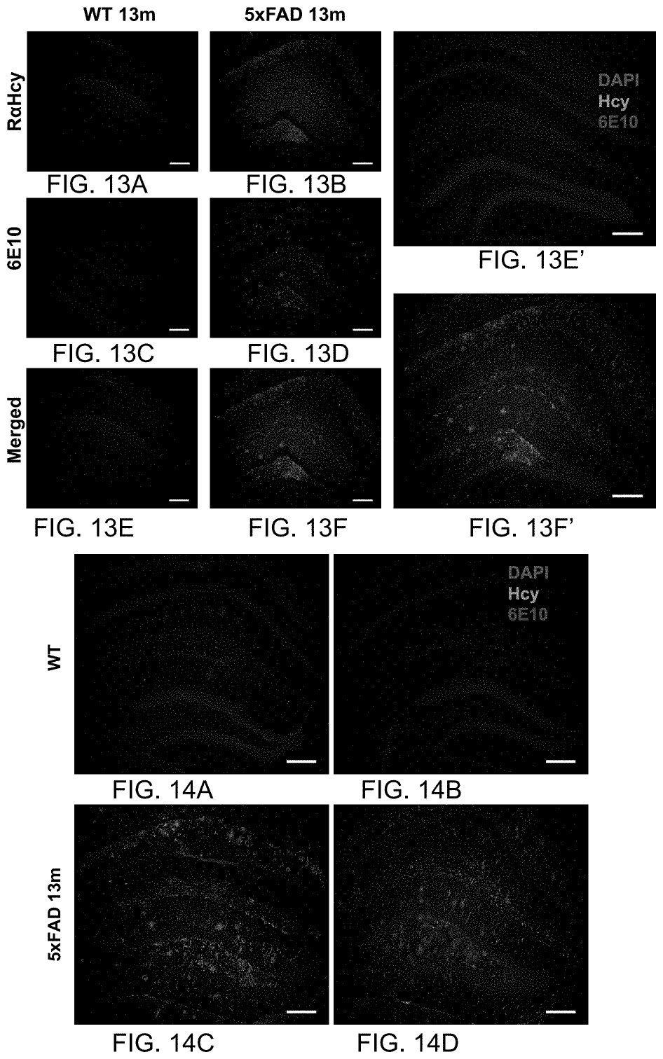

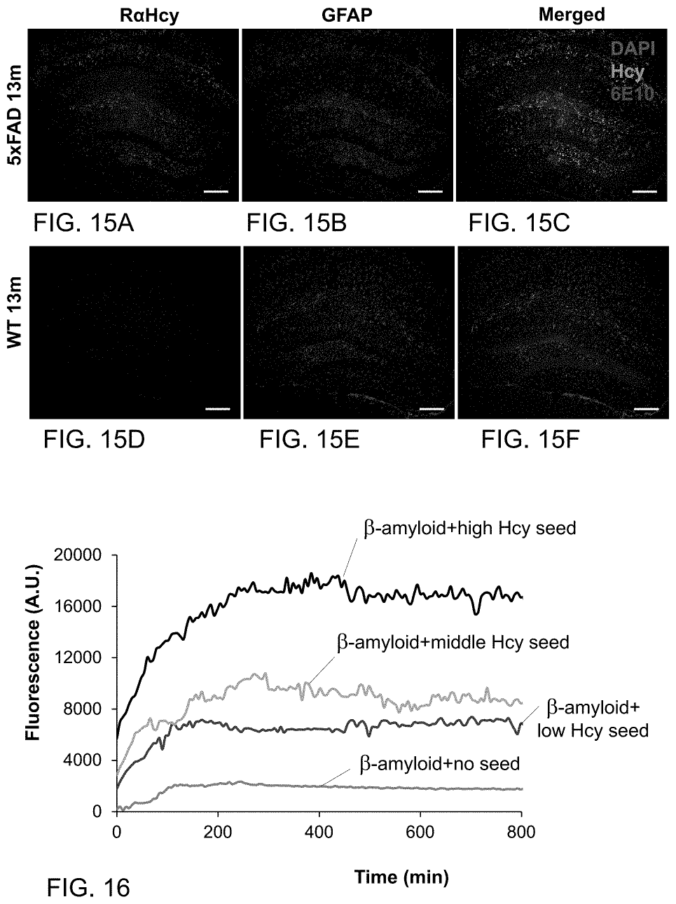

Many studies show a clear link between Hcy accumulation in serum and AD pathology, as well as Hcy toxicity towards hippocampal and cortical neurons and towards astrocytes (35-37, 63, 64). To test the presence of Hcy fibrils in-vivo and to further search for a possible cross-talk with pathological proteins, the inventors examined whether Hcy fibrils could be detected in brain sections of AD model mice. Brain sections of WT and AD model mice (5×FAD, 13m (65, 66)) were immunostained using a rabbit anti-Hcy-fibrils antibody developed by the inventors (experimental procedures) and two additional commercial anti-monomeric-Hcy antibodies (FIG. 12). More specifically, as metabolite amyloids represent a unique immunological entity with distinct epitopes, the production of antibodies against the assemblies can greatly contribute to the study of metabolite amyloids in AD pathology. The inventors successfully generated antibodies for the detection of Hcy assemblies. Using the generated antibodies, Hcy fibrils were detected in-vitro as well as in brain sections of AD mice (FIG. 12). The specificity of the generated rabbit anti-Hcy-fibrils antibody was tested towards Hcy assemblies using ELISA (FIG. 12A) and was compared to two commercial antibodies against Hcy monomers. The rabbit anti-Hcy-fibrils showed a significantly stronger signal in comparison to the commercial antibodies. Among the three antibodies, only the rabbit anti-Hcy-fibrils antibody of the present disclosure could detect Hcy in brain sections of AD model mice (FIG. 13, FIG. 12B), demonstrating the formation of Hcy assemblies in-vivo. WT sections stained with anti-Hcy antibodies did not show any signal, suggesting a clear link between Hcy fibril formation and AD pathology (FIG. 13A, FIG. 14). In parallel, the same sections were immuno-stained with anti-β-amyloid₁₋₄₂ antibody (6E10) (FIG. 13C-13F, and the corresponding FIGS. 13E′, 13F′, FIG. 12) or an antibody for anti-glial fibrillary acidic protein (GFAP), which is highly expressed in astrocytes (FIG. 15A-15F). In AD model mice, both β-amyloid₁₋₄₂ and astrocytes could be detected (FIG. 13d, FIG. 15b). Furthermore, the inventors could observe a correlation between detected Hcy fibrils and β-amyloid₁₋₄₂ aggregation (FIG. 13F) as well as co-localization of Hcy fibrils with the astrocytes (FIG. 15C), while WT sections showed a positive staining of astrocytes but no β-amyloid₁₋₄₂ signal, as expected (FIG. 13C, FIG. 15E, FIG. 14A-14D).

### Example 9

Seeding of β-amyloid₁₋₄₂ by Hcy Assemblies

Following the observed correlation in the distribution of Hcy and β-amyloid, and as the exacerbation of β-amyloid-induced toxicity by Hcy has already been reported in previous studies (35-37, 61), the ability of Hcy assemblies to directly cross-seed the aggregation of β-amyloid₁₋₄₂ was next investigated. Monomeric β-amyloid₁₋₄₂ was co-incubated with pre-formed Hcy assemblies and protein aggregation was monitored using a ThT binding assay (FIG. 16). The inventors found that Hcy seeds induced β-amyloid₁₋₄₂ aggregation in a concentration-dependent manner, with higher concentrations of the pre-formed Hcy assemblies resulting in a higher ThT signal. Samples of β-amyloid₁₋₄₂ in the absence of Hcy assemblies showed a significantly lower fluorescence signal (FIG. 16).

### Example 10

HTS Yeast Growth Assay

High throughput screens (HTS) often involve testing large numbers of compounds with high efficiency in parallel. For example, hundreds of thousands of compounds can be routinely screened in short periods, e.g., hours to days. Often, such screening is performed in multi-well plates containing, e.g., e.g., 96, 384, 1536, 3456, or more wells (sometimes referred to as micro-well or microtiter plates or dishes) or other vessels in which multiple physically separated cavities or depressions or areas are present in or on a substrate. High throughput screens can involve the use of automation, e.g., for liquid handling, imaging, data acquisition and processing, etc., as described in Macarron R & Hertzberg R P. Design and implementation of high-throughput screening assays (Methods Mol Biol., 565: 1-32, 2009). More specifically, the high-throughput screening is calibrated for 384 plate format using phenotypic assay based on yeast growth rate (measured by OD). The goal is to find hit compounds that dramatically improve the growth rate as compared to the salvage mutant in the presence of Hcy. All relevant controls are used including: mutant yeast grown without Hcy addition, WT yeast, and mutant yeast in the presence of Hcy with polyphenols (EGCG and TA), as a positive control compounds. Potential hits are selected after calculations of growth curve slopes and area under the curve during the logarithmic phase. After hit validation and dose response experiments, hits displaying low IC50 rates are selected.

### Example 11

Diagnostic Applications for Metabolite-Fibrils Antibodies

The present disclosure discloses the generation of antibodies that specifically detect fibril formation from several metabolites including homocysteine, as well as for fibrils of phenylalanine, tyrosine, tryptophan, oxalate, homocysteine and adenine [Adler-Abramovich, L. et al. Nat. Chem. Biol. 8, 701-706 (2012): Yazdi, et al. Proceedings of the National Academy of Sciences 118, no. 24 (2021); Zaguri. D. et al. Molecules 23, 1273 (2018); Laor, D. et al. Nat. Commun. (2019); Zaguri, D. et al. Molecules 23, 1273 (2018)]. More specifically, antibodies directed specifically against purified Hcy-fibrils, are disclosed herein. To purify antibodies towards anti-metabolite assemblies, the assemblies served as antigens in a series of immunization cycles in rabbits. Polyclonal IgG antibodies were purified using a protein G column chromatography.

The results of the present disclosure reveal the interplay between β-amyloid and homocysteine, and thus imply a molecular basis for the association between homocysteine accumulation and AD pathology. Thus, detection and monitoring of metabolite self-assemblies (e.g., Hcy-fibrils) in samples of patients serves as a powerful diagnostic marker for early detection of disorders associated with beta amyloid aggregations (AD).

Still further, detection is observed in body fluid samples of patients (serum, CSF), using the specific anti-Hcy-fibrils antibodies of the present disclosure, buy dot blot analysis, ELISA and any immune detection method.

1. A yeast screening system for screening of candidate therapeutic compound/s for treating, preventing, ameliorating, reducing or delaying the onset of at least one proteniopathy and/or protein misfolding disorder, said system comprises:
(a) a yeast cell and/or yeast cell line, and/or yeast cell population, that display accumulation of at least one metabolite, wherein at least one of: (i) said yeast cell/s carry at least one manipulation and/or modification in at least one yeast metabolic pathway that leads to accumulation of said metabolite; (ii) said yeast cell/s grow under conditions that result in accumulation of said metabolite; (iii) said yeast cell/s endogenously and/or exogenously express at least one pathological protein associated with said proteniopathy and/or protein misfolding disorder; and optionally
(b) at least one reagent or means for determining at least one of, the accumulation of said metabolite and at least one phenotype associated with accumulation of said metabolite and/or said pathologic protein.

2. The system according to claim 1, further comprising at least one validation means for said candidate therapeutic compound, said validation means is at least one of:
(a) at least one unicellular organism that display accumulation of said metabolite and/or accumulation of said pathological protein;
(b) at least one multicellular eukaryotic organism that display accumulation of said metabolite and/or accumulation of said pathological protein; and
(c) at least one mammalian cell that display accumulation of said metabolite and/or accumulation of said pathological protein;
(d) at least one mammalian animal model that display accumulation of said metabolite and/or accumulation of said pathological protein.

3. The system according to claim 1, wherein said phenotype associated with accumulation of said metabolite and/or accumulation of said pathological protein is at least one of cell toxicity and formation of metabolite aggregates and/or aggregates of said pathologic protein.

4. The system according to claim 1, wherein said metabolite is any one of an amino acid residue, a nucleobase, nucleoside, nucleotide, carbohydrate, fatty acid and ketone, sterols, porphyrin and haem, lipid, sphingolipid, phospholipid and lipoprotein, neurotransmitters, vitamins, non-protein cofactors, pterin, trace elements, metals, metabolites associated with energy metabolism, metabolites associated with peroxisome functions, or any intermediate product, derivative or metabolite thereof.

5. The system according to claim 4, wherein said metabolite is at least one amino acid residue, any derivative, or any intermediate product or metabolite thereof, optionally, said amino acid residue or any intermediate product or metabolite thereof is at least one of: Homocysteine (Hcy), Phenylalanine, Tyrosine, Glycine, Arginine, Cysteine, Isoleucine, Leucine, Lysine, Methionine, Proline, Tryptophane, Valine, N-acetylaspartate (NAA), Homogentisic acid, and any derivatives thereof.

6. The system according to claim 5, wherein said amino acid residue or any intermediate product or metabolite thereof is homocysteine, and wherein at least one of:
(a) said yeast cell and/or yeast cell line, and/or yeast cell population, carry a genetic and/or epigenetic modification in the Cystathionine beta-synthase 4 (CYS4) yeast gene;
(b) said genetically and/or epigenetically modified yeast cell and/or yeast cell line, and/or yeast cell population, display reduced or no expression of CYS4 gene, thereby displaying accumulation of Homocysteine and any derivative thereof; and
(c) said yeast cell and/or yeast cell line, and/or yeast cell population grow under conditions of Homocysteine supplementation.

7. The system according to claim 1, wherein said proteinopathy and/or protein misfolding and/or protein aggregation disorder is a neurodegenerative disorder or any signs or symptoms associated therewith.

8. The system according to claim 7, wherein at least one of:
(a) said proteinopathy and/or neurodegenerative disorder is a disorder characterized by at least one of beta-amyloid protein aggregation, an alpha synuclein and/or beta-synuclein aggregates, islet amyloid polypeptide aggregates, TAR DNA-binding protein 43 aggregates, huntingtin aggregates, serum amyloid protein aggregates, and tau protein aggregation;
(b) said beta-amyloid protein aggregation disorder or tauopathy is at least one of Alzheimer's disease (AD) and age-associated cognitive decline (ACD), wherein said alpha-synuclein pathology is at least one of Parkinson disease (PD), Dementia with Lewy Bodies (DLB) and multiple system atrophy (MSA), wherein said TAR DNA-binding protein 43 pathology is amyotrophic lateral sclerosis (ALS), wherein said huntingtin protein pathology is Huntington disease, wherein said islet amyloid polypeptide pathology is Type 2 diabetes, and wherein said serum amyloid protein pathology is systemic amyloidosis; and
(c) said pathological protein is at least one of beta-amyloid protein, alpha synuclein and/or beta-synuclein, islet amyloid polypeptide, TAR DNA-binding protein 43, huntingtin protein, serum amyloid proteins and tau protein.

9. The yeast screening system according to claim 1, for screening of candidate therapeutic compounds for treating, preventing, ameliorating, reducing or delaying the onset of at least one proteniopathy characterized by at least one beta-amyloid protein aggregation, said system comprising:
(a) a yeast cell and/or yeast cell line, and/or yeast cell population, that display accumulation of Homocysteine, wherein at least one of: (i) said yeast cell/s carry at least one manipulation and/or modification in at least one yeast metabolic pathway that leads to accumulation of said homocysteine (Hcy); (ii) said yeast cell/s grow under conditions that result in accumulation of said Hcy: (iii) said yeast cell/s endogenously and/or exogenously express at least one proteinopathy associated with beta-amyloid protein aggregation; and optionally
(b) at least one reagent or means for determining at least one of, the accumulation of said Hcy and/or any derivatives thereof, and/or at least one phenotype associated with accumulation of said Hcy and any derivative thereof, and/or aggregation of said beta-amyloid protein.

10. A screening method of candidate therapeutic compounds for treating, preventing, ameliorating, reducing or delaying the onset of at least one proteinopathy and/or a protein misfolding disorder, the method comprising the steps of:
(a) contacting a manipulated yeast cell and/or yeast cell line, and/or yeast cell population that display accumulation of at least one metabolite, with a candidate compound, wherein at least one of: (i) said yeast cell/s carry at least one manipulation and/or modification in at least one yeast metabolic pathway that leads to accumulation of said metabolite; (ii) said yeast cell/s grow under conditions that result in accumulation of said metabolite; (iii) said yeast cell/s endogenously and/or exogenously express at least one pathological protein associated with said proteinopathy and/or protein misfolding disorder;
(b) determining in the contacted cells of (a), at least one of: (i) the accumulation of said metabolite; (ii) at least one phenotype associated with the accumulation of said metabolite; and/or (iii) accumulation of said pathological protein; and
(c) determining that said candidate is a therapeutic compound for said proteinopathy and/or protein misfolding disorder if a modulation and/or change is detected in said cells, in at least one of: (i) the accumulation of said metabolite; (ii) accumulation of said pathological protein; and/or (iii) said phenotype, as compared with the accumulation of said metabolite, and/or accumulation of said pathological protein and/or said phenotype, in the absence of said candidate compound.

11. The method according to claim 10, further comprising the step of validating a candidate compound displaying a change and/or modulation in at least one of: the accumulation of said metabolite and/or accumulation of said pathological protein and/or said phenotype, as determined in step (c), by:
(I) contacting said candidate compound with at least one of:
(i) at least one unicellular organism that display accumulation of said metabolite and/or accumulation of said pathological protein;
(ii) at least one multicellular eukaryotic organism that display accumulation of said metabolite and/or accumulation of said pathological protein;
(iii) at least one mammalian cell that display accumulation of said metabolite and/or accumulation of said pathological protein; and
(iv) at least one mammalian animal model that display accumulation of said metabolite and/or accumulation of said pathological protein;
(II) determining in the cells, unicellular organism, multicellular organism or mammal of (I), at least one phenotype associated with the accumulation of said metabolite and/or accumulation g of said pathological protein; and
(III) determining that said candidate is a therapeutic compound for said protein misfolding disorder if a change and/or modulation is detected in at least one of: the accumulation of said metabolite and/or accumulation of said pathological protein, and/or said phenotype, as compared with the level of at least one of: the accumulation of said metabolite, and/or accumulation of said pathological protein, and/or the phenotype, in the absence of said candidate compound, optionally, said phenotype associated with accumulation of said metabolite and/or accumulation of said pathological protein is at least one of cell toxicity and formation of metabolite aggregate.

12. The method according to claim 10, wherein said metabolite is any one of an amino acid residue, a nucleobase, nucleoside, nucleotide, carbohydrate, fatty acid and ketone, sterols, porphyrin and haem, lipid, sphingolipid, phospholipid and lipoprotein, neurotransmitters, vitamins and (non-protein) cofactors, pterin, trace elements, metals, metabolites associated with energy metabolism, metabolites associated with peroxisome functions, or any intermediate product, derivative or metabolite thereof.

13. The method according to claim 12, wherein said metabolite is at least one amino acid residue, any derivative, or any intermediate product or metabolite thereof, optionally, said amino acid residue or any intermediate product or metabolite thereof is at least one of: Homocysteine, Phenylalanine, Tyrosine, Glycine, Arginine, Cysteine, Isoleucine, Leucine, Lysine, Methionine, Proline, Tryptophane, Valine, N-acetylaspartate (NAA), Homogentisic acid, and any derivatives thereof.

14. The method according to claim 13, wherein said amino acid residue or any intermediate product or metabolite thereof is Homocysteine, and wherein at least one of:
(a) said yeast cell and/or yeast cell line, and/or yeast cell population, carry a genetic and/or epigenetic modification in the Cystathionine beta-synthase 4 (CYS4) yeast gene;
(b) said genetically and/or epigenetically modified yeast cell and/or yeast cell line, and/or yeast cell population, display reduced or no expression of CYS4 gene, thereby displaying accumulation of Homocysteine and any derivative thereof, and
(c) said yeast cell and/or yeast cell line, and/or yeast cell population grow under conditions of Homocysteine supplementation.

15. The method according to claim 14, wherein said proteinopathy and/or protein misfolding disorder is a proteinopathy and/or a neurodegenerative disorder or any signs or symptoms associated therewith, and wherein at least one of:
(a) said neurodegenerative disorder is a disorder characterized by at least one of beta-amyloid protein aggregation, an alpha synuclein and/or beta-synuclein aggregates and tau protein aggregation;
(b) said beta-amyloid protein aggregation disorder or tauopathy is at least one of Alzheimer's disease (AD) and age-associated cognitive decline (ACD), wherein said alpha-synuclein pathology is at least one of Parkinson disease (PD), Dementia with Lewy Bodies (DLB) and multiple system atrophy (MSA), wherein said TAR DNA-binding protein 43 pathology is amyotrophic lateral sclerosis (ALS), wherein said huntingtin protein pathology is Huntington disease, wherein said islet amyloid polypeptide pathology is Type 2 diabetes, and wherein said serum amyloid protein pathology is systemic amyloidosis; and
(c) said pathological protein is at least one of beta-amyloid protein, alpha synuclein, beta-synuclein and tau protein.

16. The screening method according to claim 10, for screening of a candidate therapeutic compounds for treating, preventing, ameliorating, reducing or delaying the onset of a proteinopathy characterized by at least one beta-amyloid protein aggregation, the method comprising the steps of:
(a) contacting a yeast cell and/or yeast cell line, and/or yeast cell population, that display accumulation of Homocysteine, with a candidate compound, wherein at least one of: (i) said yeast cell/s carry at least one manipulation in at least one yeast metabolic pathway that leads to accumulation of said homocysteine (Hcy); (ii) said yeast cell/s grow under conditions that result in accumulation of said Hcy; (iii) said yeast cell/s endogenously and/or exogenously express said beta-amyloid protein or any pathological protein associated with said proteinopathy;
(b) determining in the contacted cells of (a) at least one of: the accumulation of said Hcy and any derivative thereof, and/or beta-amyloid protein aggregation, and/or at least one phenotype associated with said accumulation of said at least one of Hcy and/or beta-amyloid protein aggregation said; and
(c) determining that said candidate is a therapeutic compound for said proteinopathy characterized by at least one of beta-amyloid protein aggregation if a change or modulation is detected in at least one of: (i) the accumulation of said Hcy; (ii) beta-amyloid protein aggregation; and/or (iii) said phenotype, as compared with the accumulation of said Hcy and/or said beta-amyloid protein aggregation, and/or said phenotype, in the absence of said candidate compound.

17. A method for treating, preventing, ameliorating, reducing or delaying the onset of at least one proteinopathy and/or protein misfolding disorder, the method comprising the steps of:
(I) obtaining a compound that modulates the level of at least one phenotype associated with the accumulation of at least one metabolite by the screening method according to claim 10; and
(II) administering a therapeutic effective amount of the compound obtained by step (I) to a subject suffering from said at least one protein misfolding disorder.

18. A therapeutic compound for treating, preventing, ameliorating, reducing or delaying the onset of at least one protein misfolding disorder, wherein said compound is identified by the screening method as defined by claim 10, and wherein said compound is at least one of a small molecule, aptamer, a peptide, a nucleic acid molecule and an immunological agent, and any combinations thereof.

19. A method for detection, and monitoring of at least one protein misfolding disorder or proteniopathy in a subject, the method comprising:
(a) contacting at least one biological sample of said subject with at least one antibody specific for at least one metabolite-fibrils;
(b) classifying said subject as a subject affected by said at least one protein misfolding disorder or proteniopathy, if said metabolite-fibril is detected in the sample; and optionally
(c) administering to a subject classified as affected by said at least one protein misfolding disorder or proteniopathy, a therapeutically effective amount of at least one anti-proteinopathy or an anti-protein misfolding disorder therapeutic agent.

20. A method for treating, preventing, ameliorating, reducing or delaying the onset of at least one proteinopathy and/or protein misfolding disorder, the method comprising the steps of:
(a) contacting at least one biological sample of said subject with at least one antibody specific for at least one metabolite-fibrils;
(b) classifying said subject as a subject affected by said at least one protein misfolding disorder or proteniopathy, if said metabolite-fibril is detected in the sample; and
(c) administering to a subject classified as affected by said at least one protein misfolding disorder or proteniopathy, a therapeutically effective amount of at least one anti-proteinopathy or an anti-protein misfolding disorder therapeutic agent.


SEQUENCE LISTING (15 sequences)

SEQ ID NO: 1 - DNA, 80 bp, Artificial Sequence
  Primer DL154
gatttcgttgtaggccacttgctcaaaggacatctagataaatacgacgtaagaataaaacggatccccgggttaattaa

SEQ ID NO: 2 - DNA, 80 bp, Artificial Sequence
  Primer DL155
aggaaaggaatgacggattttgcttctatgtttgcttttatttgaagcgtgggttcttatacatttgattaaaatagaac

SEQ ID NO: 3 - DNA, 1524 bp, Saccharomyces cerevisiae
atgactaaatctgagcagcaagccgattcaagacataacgttatcgacttagttggtaacaccccattgatcgcactgaaaaaattgcctaaggctttgggtatcaaaccacaaatttatgctaagctggaactatacaatccaggtggttccatcaaagacagaattgccaagtctatggtggaagaagctgaagcttccggtagaattcatccttccagatctactctgatcgaacctacttctggtaacaccggtatcggtctagctttaatcggcgccatcaaaggttacagaactatcatcaccttgccggaaaaaatgtctaacgagaaagtttctgtcctaaaggctctgggtgctgaaatcatcagaactccaactgctgctgcctgggattctccagaatcacatattggtgttgctaagaagttggaaaaagagattcctggtgctgttatacttgaccaatataacaatatgatgaacccagaagctcattactttggtactggtcgcgaaatccaaagacagctagaagacttgaatttatttgataatctacgcgctgttgttgctggtgctggtactggtgggactattagcggtatttccaagtacttgaaagaacagaatgataagatccaaatcgttggtgctgacccattcggttcaattttagcccaacctgaaaacttgaataagactgatatcactgactacaaagttgagggtattggttatgattttgttcctcaggttttggacagaaaattaattgatgtttggtataagacagacgacaagccttctttcaaatacgccagacaattgatttctaacgaaggtgtcttggtgggtggttcttccggttctgccttcactgcggttgtgaaatactgtgaagaccaccctgaactgactgaagatgatgtcattgttgccatattcccagattccatcaggtcgtacctaaccaaattcgtcgatgacgaatggttgaaaaagaacaatttgtgggatgatgacgtgttggcccgttttgactcttcaaagctggaggcttcgacgacaaaatacgctgatgtgtttggtaacgctactgtaaaggatcttcacttgaaaccggttgtttccgttaaggaaaccgctaaggtcactgatgttatcaagatattaaaagacaatggctttgaccaattgcctgtgttgactgaagacggcaagttgtctggtttagttactctctctgagcttctaagaaaactatcaatcaataattcaaacaacgacaacactataaagggtaaatacttggacttcaagaaattaaacaatttcaatgatgtttcctcttacaacgaaaataaatccggtaagaagaagtttattaaattcgatgaaaactcaaagctatctgacttgaatcgtttctttgaaaaaaactcatctgccgttatcactgatggcttgaaaccaatccatatcgttactaagatggatttactgagctacttagcataa

SEQ ID NO: 4 - protein, 507 aa, Saccharomyces cerevisiae
MTKSEQQADSRHNVIDLVGNTPLIALKKLPKALGIKPQIYAKLELYNPGGSIKDRIAKSMVEEAEASGRIHPSRSTLIEPTSGNTGIGLALIGAIKGYRTIITLPEKMSNEKVSVLKALGAEIIRTPTAAAWDSPESHIGVAKKLEKEIPGAVILDQYNNMMNPEAHYFGTGREIQRQLEDLNLFDNLRAVVAGAGTGGTISGISKYLKEQNDKIQIVGADPFGSILAQPENLNKTDITDYKVEGIGYDFVPQVLDRKLIDVWYKTDDKPSFKYARQLISNEGVLVGGSSGSAFTAVVKYCEDHPELTEDDVIVAIFPDSIRSYLTKFVDDEWLKKNNLWDDDVLARFDSSKLEASTTKYADVFGNATVKDLHLKPVVSVKETAKVTDVIKILKDNGFDQLPVLTEDGKLSGLVTLSELLRKLSINNSNNDNTIKGKYLDFKKLNNFNDVSSYNENKSGKKKFIKFDENSKLSDLNRFFEKNSSAVITDGLKPIHIVTKMDLLSYLA

SEQ ID NO: 5 - protein, 770 aa, Homo sapiens
MLPGLALLLLAAWTARALEVPTDGNAGLLAEPQIAMFCGRLNMHMNVQNGKWDSDPSGTKTCIDTKEGILQYCQEVYPELQITNVVEANQPVTIQNWCKRGRKQCKTHPHFVIPYRCLVGEFVSDALLVPDKCKFLHQERMDVCETHLHWHTVAKETCSEKSTNLHDYGMLLPCGIDKFRGVEFVCCPLAEESDNVDSADAEEDDSDVWWGGADTDYADGSEDKVVEVAEEEEVAEVEEEEADDDEDDEDGDEVEEEAEEPYEEATERTTSIATTTTTTTESVEEVVREVCSEQAETGPCRAMISRWYFDVTEGKCAPFFYGGCGGNRNNFDTEEYCMAVCGSAMSQSLLKTTQEPLARDPVKLPTTAASTPDAVDKYLETPGDENEHAHFQKAKERLEAKHRERMSQVMREWEEAERQAKNLPKADKKAVIQHFQEKVESLEQEAANERQQLVETHMARVEAMLNDRRRLALENYITALQAVPPRPRHVFNMLKKYVRAEQKDRQHTLKHFEHVRMVDPKKAAQIRSQVMTHLRVIYERMNQSLSLLYNVPAVAEEIQDEVDELLQKEQNYSDDVLANMISEPRISYGNDALMPSLTETKTTVELLPVNGEFSLDDLQPWHSFGADSVPANTENEVEPVDARPAADRGLTTRPGSGLTNIKTEEISEVKMDAEFRHDSGYEVHHQKLVFFAEDVGSNKGAIIGLMVGGVVIATVIVITLVMLKKKQYTSIHHGVVEVDAAVTPEERHLSKMQQNGYENPTYKFFEQMQN

SEQ ID NO: 6 - protein, 758 aa, Homo sapiens
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQEPESGKVVQEGFLREPGPPGLSHQLMSGMPGAPLLPEGPREATRQPSGTGPEDTEGGRHAPELLKHQLLGDLHQEGPPLKGAGGKERPGSKEEVDEDRDVDESSPQDSPPSKASPAQDGRPPQTAAREATSIPGFPAEGAIPLPVDFLSKVSTEIPASEPDGPSVGRAKGQDAPLEFTFHVEITPNVQKEQAHSEEHLGRAAFPGAPGEGPEARGPSLGEDTKEADLPEPSEKQPAAAPRGKPVSRVPQLKARMVSKSKDGTGSDDKKAKTSTRSSAKTLKNRPCLSPKHPTPGSSDPLIQPSSPAVCPEPPSSPKYVSSVTSRTGSSGAKEMKLKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 7 - protein, 441 aa, Homo sapiens
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 8 - protein, 140 aa, Homo sapiens
MDVFMKGLSKAKEGVVAAAEKTKQGVAEAAGKTKEGVLYVGSKTKEGVVHGVATVAEKTKEQVTNVGGAVVTGVTAVAQKTVEGAGSIAAATGFVKKDQLGKNEEGAPQEGILEDMPVDPDNEAYEMPSEEGYQDYEPEA

SEQ ID NO: 9 - protein, 134 aa, Homo sapiens
MDVFMKGLSMAKEGVVAAAEKTKQGVTEAAEKTKEGVLYVGSKTREGVVQGVASVAEKTKEQASHLGGAVFSGAGNIAAATGLVKREEFPTDLKPEEVAQEAAEEPLIEPLMEPEGESYEDPPQEEYQEYEPEA

SEQ ID NO: 10 - protein, 3142 aa, Homo sapiens
MATLEKLMKAFESLKSFQQQQQQQQQQQQQQQQQQQQQPPPPPPPPPPPQLPQPPPQAQPLLPQPQPPPPPPPPPPGPAVAEEPLHRPKKELSATKKDRVNHCLTICENIVAQSVRNSPEFQKLLGIAMELFLLCSDDAESDVRMVADECLNKVIKALMDSNLPRLQLELYKEIKKNGAPRSLRAALWRFAELAHLVRPQKCRPYLVNLLPCLTRTSKRPEESVQETLAAAVPKIMASFGNFANDNEIKVLLKAFIANLKSSSPTIRRTAAGSAVSICQHSRRTQYFYSWLLNVLLGLLVPVEDEHSTLLILGVLLTLRYLVPLLQQQVKDTSLKGSFGVTRKEMEVSPSAEQLVQVYELTLHHTQHQDHNVVTGALELLQQLFRTPPPELLQTLTAVGGIGQLTAAKEESGGRSRSGSIVELIAGGGSSCSPVLSRKQKGKVLLGEEEALEDDSESRSDVSSSALTASVKDEISGELAASSGVSTPGSAGHDIITEQPRSQHTLQADSVDLASCDLTSSATDGDEEDILSHSSSQVSAVPSDPAMDLNDGTQASSPISDSSQTTTEGPDSAVTPSDSSEIVLDGTDNQYLGLQIGQPQDEDEEATGILPDEASEAFRNSSMALQQAHLLKNMSHCRQPSDSSVDKFVLRDEATEPGDQENKPCRIKGDIGQSTDDDSAPLVHCVRLLSASFLLTGGKNVLVPDRDVRVSVKALALSCVGAAVALHPESFFSKLYKVPLDTTEYPEEQYVSDILNYIDHGDPQVRGATAILCGTLICSILSRSRFHVGDWMGTIRTLTGNTFSLADCIPLLRKTLKDESSVTCKLACTAVRNCVMSLCSSSYSELGLQLIIDVLTLRNSSYWLVRTELLETLAEIDFRLVSFLEAKAENLHRGAHHYTGLLKLQERVLNNVVIHLLGDEDPRVRHVAAASLIRLVPKLFYKCDQGQADPVVAVARDQSSVYLKLLMHETQPPSHFSVSTITRIYRGYNLLPSITDVTMENNLSRVIAAVSHELITSTTRALTFGCCEALCLLSTAFPVCIWSLGWHCGVPPLSASDESRKSCTVGMATMILTLLSSAWFPLDLSAHQDALILAGNLLAASAPKSLRSSWASEEEANPAATKQEEVWPALGDRALVPMVEQLFSHLLKVINICAHVLDDVAPGPAIKAALPSLTNPPSLSPIRRKGKEKEPGEQASVPLSPKKGSEASAASRQSDTSGPVTTSKSSSLGSFYHLPSYLKLHDVLKATHANYKVTLDLQNSTEKFGGFLRSALDVLSQILELATLQDIGKCVEEILGYLKSCFSREPMMATVCVQQLLKTLFGTNLASQFDGLSSNPSKSQGRAQRLGSSSVRPGLYHYCFMAPYTHFTQALADASLRNMVQAEQENDTSGWFDVLQKVSTQLKTNLTSVTKNRADKNAIHNHIRLFEPLVIKALKQYTTTTCVQLQKQVLDLLAQLVQLRVNYCLLDSDQVFIGFVLKQFEYIEVGQFRESEAIIPNIFFFLVLLSYERYHSKQIIGIPKIIQLCDGIMASGRKAVTHAIPALQPIVHDLFVLRGTNKADAGKELETQKEVVVSMLLRLIQYHQVLEMFILVLQQCHKENEDKWKRLSRQIADIILPMLAKQQMHIDSHEALGVLNTLFEILAPSSLRPVDMLLRSMFVTPNTMASVSTVQLWISGILAILRVLISQSTEDIVLSRIQELSFSPYLISCTVINRLRDGDSTSTLEEHSEGKQIKNLPEETFSRFLLQLVGILLEDIVTKQLKVEMSEQQHTFYCQELGTLLMCLIHIFKSGMFRRITAAATRLFRSDGCGGSFYTLDSLNLRARSMITTHPALVLLWCQILLLVNHTDYRWWAEVQQTPKRHSLSSTKLLSPQMSGEEEDSDLAAKLGMCNREIVRRGALILFCDYVCQNLHDSEHLTWLIVNHIQDLISLSHEPPVQDFISAVHRNSAASGLFIQAIQSRCENLSTPTMLKKTLQCLEGIHLSQSGAVLTLYVDRLLCTPFRVLARMVDILACRRVEMLLAANLQSSMAQLPMEELNRIQEYLQSSGLAQRHQRLYSLLDRFRLSTMQDSLSPSPPVSSHPLDGDGHVSLETVSPDKDWYVHLVKSQCWTRSDSALLEGAELVNRIPAEDMNAFMMNSEFNLSLLAPCLSLGMSEISGGQKSALFEAAREVTLARVSGTVQQLPAVHHVFQPELPAEPAAYWSKLNDLFGDAALYQSLPTLARALAQYLVVVSKLPSHLHLPPEKEKDIVKFVVATLEALSWHLIHEQIPLSLDLQAGLDCCCLALQLPGLWSVVSSTEFVTHACSLIYCVHFILEAVAVQPGEQLLSPERRTNTPKAISEEEEEVDPNTQNPKYITAACEMVAEMVESLQSVLALGHKRNSGVPAFLTPLLRNIIISLARLPLVNSYTRVPPLVWKLGWSPKPGGDFGTAFPEIPVEFLQEKEVFKEFIYRINTLGWTSRTQFEETWATLLGVLVTQPLVMEQEESPPEEDTERTQINVLAVQAITSLVLSAMTVPVAGNPAVSCLEQQPRNKPLKALDTRFGRKLSIIRGIVEQEIQAMVSKRENIATHHLYQAWDPVPSLSPATTGALISHEKLLLQINPERELGSMSYKLGQVSIHSVWLGNSITPLREEEWDEEEEEEADAPAPSSPPTSPVNSRKHRAGVDIHSCSQFLLELYSRWILPSSSARRTPAILISEVVRSLLVVSDLFTERNQFELMYVTLTELRRVHPSEDEILAQYLVPATCKAAAVLGMDKAVAEPVSRLLESTLRSSHLPSRVGALHGVLYVLECDLLDDTAKQLIPVISDYLLSNLKGIAHCVNIHSQQHVLVMCATAFYLIENYPLDVGPEFSASIIQMCGVMLSGSEESTPSIIYHCALRGLERLLLSEQLSRLDAESLVKLSVDRVNVHSPHRAMAALGLMLTCMYTGKEKVSPGRTSDPNPAAPDSESVIVAMERVSVLFDRIRKGFPCEARVVARILPQFLDDFFPPQDIMNKVIGEFLSNQQPYPQFMATVVYKVFQTLHSTGQSSMVRDWVMLSLSNFTQRAPVAMATWSLSCFFVSASTSPWVAAILPHVISRMGKLEQVDVNLFCLVATDFYRHQIEEELDRRAFQSVLEVVAAPGSPYHRLLTCLRNVHKVTTC

SEQ ID NO: 11 - protein, 361 aa, Homo sapiens
MESIFHEKQEGSLCAQHCLNNLLQGEYFSPVELSSIAHQLDEEERMRMAEGGVTSEDYRTFLQQPSGNMDDSGFFSIQVISNALKVWGLELILFNSPEYQRLRIDPINERSFICNYKEHWFTVRKLGKQWFNLNSLLTGPELISDTYLALFLAQLQQEGYSIFVVKGDLPDCEADQLLQMIRVQQMHRPKLIGEELAQLKEQRVHKTDLERVLEANDGSGMLDEDEEDLQRALALSRQEIDMEDEEADLRRAIQLSMQGSSRNISQDMTQTSGTNLTSEELRKRREAYFEKQQQKQQQQQQQQQQGDLSGQSSHPCERPATSSGALGSDLGDAMSEEDMLQAAVTMSLETVRNDLKTEGKK

SEQ ID NO: 12 - protein, 2527 aa, Homo sapiens
MASGSCQGCEEDEETLKKLIVRLNNVQEGKQIETLVQILEDLLVFTYSERASKLFQGKNIHVPLLIVLDSYMRVASVQQVGWSLLCKLIEVCPGTMQSLMGPQDVGNDWEVLGVHQLILKMLTVHNASVNLSVIGLKTLDLLLTSGKITLLILDEESDIFMLIFDAMHSFPANDEVQKLGCKALHVLFERVSEEQLTEFVENKDYMILLSALTNFKDEEEIVLHVLHCLHSLAIPCNNVEVLMSGNVRCYNIVVEAMKAFPMSERIQEVSCCLLHRLTLGNFFNILVLNEVHEFVVKAVQQYPENAALQISALSCLALLTETIFLNQDLEEKNENQENDDEGEEDKLFWLEACYKALTWHRKNKHVQEAACWALNNLLMYQNSLHEKIGDEDGHFPAHREVMLSMLMHSSSKEVFQASANALSTLLEQNVNFRKILLSKGIHLNVLELMQKHIHSPEVAESGCKMLNHLFEGSNTSLDIMAAVVPKILTVMKRHETSLPVQLEALRAILHFIVPGMPEESREDTEFHHKLNMVKKQCFKNDIHKLVLAALNRFIGNPGIQKCGLKVISSIVHFPDALEMLSLEGAMDSVLHTLQMYPDDQEIQCLGLSLIGYLITKKNVFIGTGHLLAKILVSSLYRFKDVAEIQTKGFQTILAILKLSASFSKLLVHHSFDLVIFHQMSSNIMEQKDQQFLNLCCKCFAKVAMDDYLKNVMLERACDQNNSIMVECLLLLGADANQAKEGSSLICQVCEKESSPKLVELLLNSGSREQDVRKALTISIGKGDSQIISLLLRRLALDVANNSICLGGFCIGKVEPSWLGPLFPDKTSNLRKQTNIASTLARMVIRYQMKSAVEEGTASGSDGNFSEDVLSKFDEWTFIPDSSMDSVFAQSDDLDSEGSEGSFLVKKKSNSISVGEFYRDAVLQRCSPNLQRHSNSLGPIFDHEDLLKRKRKILSSDDSLRSSKLQSHMRHSDSISSLASEREYITSLDLSANELRDIDALSQKCCISVHLEHLEKLELHQNALTSFPQQLCETLKSLTHLDLHSNKFTSFPSYLLKMSCIANLDVSRNDIGPSVVLDPTVKCPTLKQFNLSYNQLSFVPENLTDVVEKLEQLILEGNKISGICSPLRLKELKILNLSKNHISSLSENFLEACPKVESFSARMNFLAAMPFLPPSMTILKLSQNKFSCIPEAILNLPHLRSLDMSSNDIQYLPGPAHWKSLNLRELLFSHNQISILDLSEKAYLWSRVEKLHLSHNKLKEIPPEIGCLENLTSLDVSYNLELRSFPNEMGKLSKIWDLPLDELHLNFDFKHIGCKAKDIIRFLQQRLKKAVPYNRMKLMIVGNTGSGKTTLLQQLMKTKKSDLGMQSATVGIDVKDWPIQIRDKRKRDLVLNVWDFAGREEFYSTHPHFMTQRALYLAVYDLSKGQAEVDAMKPWLFNIKARASSSPVILVGTHLDVSDEKQRKACMSKITKELLNKRGFPAIRDYHFVNATEESDALAKLRKTIINESLNFKIRDQLVVGQLIPDCYVELEKIILSERKNVPIEFPVIDRKRLLQLVRENQLQLDENELPHAVHFLNESGVLLHFQDPALQLSDLYFVEPKWLCKIMAQILTVKVEGCPKHPKGIISRRDVEKFLSKKRKFPKNYMSQYFKLLEKFQIALPIGEEYLLVPSSLSDHRPVIELPHCENSEIIIRLYEMPYFPMGFWSRLINRLLEISPYMLSGRERALRPNRMYWRQGIYLNWSPEAYCLVGSEVLDNHPESFLKITVPSCRKGCILLGQVVDHIDSLMEEWFPGLLEIDICGEGETLLKKWALYSFNDGEEHQKILLDDLMKKAEEGDLLVNPDQPRLTIPISQIAPDLILADLPRNIMLNNDELEFEQAPEFLLGDGSFGSVYRAAYEGEEVAVKIFNKHTSLRLLRQELVVLCHLHHPSLISLLAAGIRPRMLVMELASKGSLDRLLQQDKASLTRTLQHRIALHVADGLRYLHSAMIIYRDLKPHNVLLFTLYPNAAIIAKIADYGIAQYCCRMGIKTSEGTPGFRAPEVARGNVIYNQQADVYSFGLLLYDILTTGGRIVEGLKFPNEFDELEIQGKLPDPVKEYGCAPWPMVEKLIKQCLKENPQERPTSAQVFDILNSAELVCLTRRILLPKNVIVECMVATHHNSRNASIWLGCGHTDRGQLSFLDLNTEGYTSEEVADSRILCLALVHLPVEKESWIVSGTQSGTLLVINTEDGKKRHTLEKMTDSVTCLYCNSFSKQSKQKNFLLVGTADGKLAIFEDKTVKLKGAAPLKILNIGNVSTPLMCLSESTNSTERNVMWGGCGTKIFSFSNDFTIQKLIETRTSQLFSYAAFSDSNIITVVVDTALYIAKQNSPVVEVWDKKTEKLCGLIDCVHFLREVMVKENKESKHKMSYSGRVKTLCLQKNTALWIGTGGGHILLLDLSTRRLIRVIYNFCNSVRVMMTAQLGSLKNVMLVLGYNRKNTEGTQKQKEIQSCLTVWDINLPHEVQNLEKHIEVRKELAEKMRRTSVE

SEQ ID NO: 13 - protein, 414 aa, Homo sapiens
MSEYIRVTEDENDEPIEIPSEDDGTVLLSTVTAQFPGACGLRYRNPVSQCMRGVRLVEGILHAPDAGWGNLVYVVNYPKDNKRKMDETDASSAVKVKRAVQKTSDLIVLGLPWKTTEQDLKEYFSTFGEVLMVQVKKDLKTGHSKGFGFVRFTEYETQVKVMSQRHMIDGRWCDCKLPNSKQSQDEPLRSRKVFVGRCTEDMTEDELREFFSQYGDVMDVFIPKPFRAFAFVTFADDQIAQSLCGEDLIIKGISVHISNAEPKHNSNRQLERSGRFGGNPGGFGNQGGFGNSRGGGAGLGNNQGSNMGGGMNFGAFSINPAMMAAAQAALQSSWGMMGMLASQQNQSGPSGNNQNQGNMQREPNQAFGSGNNSYSGSNSGAAIGWGSASNAGSGSGFNGGFGSSMDSKSSGWGM

SEQ ID NO: 14 - protein, 154 aa, Homo sapiens [sequence omitted]

SEQ ID NO: 15 - protein, 89 aa, Homo sapiens
MGILKLQVFLIVLSVALNHLKATPIESHQVEKRKCNTATCATQRLANFLVHSSNNFGAILSSTNVGSNTYGKRNAVEVLKREPLNYLPL